SEQUENCE LISTING (39 sequences)

SEQ ID NO: 1 - DNA, 2016 bp, Gallus gallus
atgggctcgtgcggggcgctgggcctggggctgctgctcgccgccgtgtgcggggcggcggccggtgagtgcggccggaccgggacccctccccgcccataaccccaccccggggctgtgtccgtgggatcctcagacccccacccgcggctcacggccccgctgcgctccgtccccgcagagctccataccctgcggtacatccgtacggcgatgacggatcccggccccgggctgccgtggttcgtggacgtggggtacgtggacggggaactcttcatgcactacaacagcaccgctcggagggctgtgccccgcaccgagtggatagcggccaacacggaccagcagtactgggacagagagacgcagatcgtacagggcagtgagcagattaaccgcgagaacctggacatactgcggcggcgctacaaccagaccggcggtgagcacggccggggccgcggctccgtgggtgtgggatgggctccatggcgcagtgccgcccacaccccccaggcctggccctgcccggcggcaccgtcccggggctgcccgtcacagccccaccgcgctcggggtgccgcgtcccggggggaccccaacccatccccgctgcagtgggagccccggagccggaggggcccctcaccccctgcccggctgtgtttcagggtctcacacagtgcagtggatgtccggctgtgacatcctcgaggatggcaccatccgggggtatcatcaggcagcctacgatgggagagacttcgttgccttcgacaaaggcacgatgacgttaactgcggcagttccagaggcagttcccaccaagaggaaatgggaggaaggaggttatgctgaggggctgaagcagtacctggaggaaacctgcgtggagtggctgcggagatatgtggaatacgggaaggctgagctgggcaggagaggtgagcggagtggggggggccgcagtgtggggctggacgtggggcgggggctgagcgtggggagctcagcccggccctcactgccgcccgcccgcagagcgacccgaggtgcgagtgtgggggaaggaggccgacgggatcctgaccttgtcctgccgcgctcacggcttctacccgcggcccatcgttgtcagctggctgaaggacggcgcggtgcggggccaggacgcccagtcggggggcatcgtgcccaacggcgacggcacgtaccacacctgggtcaccatcgatgcgcagccgggggacggggacaagtaccagtgccgcgtggagcacgccagcctgccccagcccggcctctactcgtggggtgagtgaggggatgtggggctggggggctgcgggctgccccttcccctgctgatggccccgctctcccccagagccgccacagcccaacctggtgcccatcgtggcgggggtggctgtcgccattgtggccatcgccatcgtggttggtgttggattcatcatctacagacgccatgcaggtaaaagcagaggggtgcaggcgggccgtgggggcagtgggggcatctgggtccccctagggagccctcaacctggctgtgatgtgaacctgtgttgattcatctctctgtctgcagggaagaaggggaagggctacaacatcgcgcccggtgagtgatgagggcagcgctgtcccccacctctgcccagtgcccgggcggtcctggggtctccactttctctcagggtacccattcctggtgcttggggctgctccatgccccatagggagcacaggactgggtctcacagctgttcctcccttatagacagggaaggtggatccagcagctcgagcacaggtgcggtgtggggctgtgggttgggaggggtccatgtgctctcggtggtactgcccagggctgggctatgctgggactctgcggggagacccccggagcagagggttgggatgtgaacctggccccgtgggacatcatcccttctcatccccacagggagcaacccctccatctga

SEQ ID NO: 2 - DNA, 1262 bp, Gallus gallus
cttgagagtgcagcggtgcgaggcgatgggctcgtgcggggcgctgggcctggggctgctgctcgccgccgtgtgcggggcggcggccgagctccataccctgcggtacatccgtacggcgatgacggatcccggccccgggctgccgtggttcgtggacgtggggtacgtggacggggaactcttcatgcactacaacagcaccgctcggagggctgtgccccgcaccgagtggatagcggccaacacggaccagcagtactgggacagagagacgcagatcgtacagggcagtgagcagattaaccgcgagaacctggacatactgcggcggcgctacaaccagaccggcgggtctcacacagtgcagtggatgtccggctgtgacatcctcgaggatggcaccatccgggggtatcatcaggcagcctacgatgggagagacttcgttgccttcgacaaaggcacgatgacgttaactgcggcagttccagaggcagttcccaccaagaggaaatgggaggaaggaggttatgctgaggggctgaagcagtacctggaggaaacctgcgtggagtggctgcggagatatgtggaatacgggaaggctgagctgggcaggagagagcgacccgaggtgcgagtgtgggggaaggaggccgacgggatcctgaccttgtcctgccgcgctcacggcttctacccgcggcccatcgttgtcagctggctgaaggacggcgcggtgcggggccaggacgcccagtcggggggcatcgtgcccaacggcgacggcacgtaccacacctgggtcaccatcgatgcgcagccgggggacggggacaagtaccagtgccgcgtggagcacgccagcctgccccagcccggcctctactcgtgggagccgccacagcccaacctggtgcccatcgtggcgggggtggctgtcgccattgtggccatcgccatcgtggttggtgttggattcatcatctacagacgccatgcagggaagaaggggaagggctacaacatcgcgcccgacagggaaggtggatccagcagctcgagcacagggagcaacccctccatctgagtgctgtgcttcagcctgcaaggagccaacagtccacaccagcatttggggtcggtgatggacacagccccatcctcttgacctctcagatgtccctctgcttcctatgctgactgttattctttgcctgcactgcttcctgtgaaataaaatgatgggccattctgtg

SEQ ID NO: 3 - protein, 355 aa, Gallus gallus
  mat_peptide (22)..()
MGSCGALGLGLLLAAVCGAAAELHTLRYIRTAMTDPGPGLPWFVDVGYVDGELFMHYNSTARRAVPRTEWIAANTDQQYWDRETQIVQGSEQINRENLDILRRRYNQTGGSHTVQWMSGCDILEDGTIRGYHQAAYDGRDFVAFDKGTMTLTAAVPEAVPTKRKWEEGGYAEGLKQYLEETCVEWLRRYVEYGKAELGRRERPEVRVWGKEADGILTLSCRAHGFYPRPIVVSWLKDGAVRGQDAQSGGIVPNGDGTYHTWVTIDAQPGDGDKYQCRVEHASLPQPGLYSWEPPQPNLVPIVAGVAVAIVAIAIVVGVGFIIYRRHAGKKGKGYNIAPDREGGSSSSSTGSNPSI

SEQ ID NO: 4 - DNA, 2034 bp, Gallus gallus
atgcgcccgtgcggggcggtgggcctggggctgctgcgcctggggctgctgctcgccgccgtgtgcggggcggcggccggtgagtgcggccggaccgggacccctccccgcccgtaaccccaccccggggctgtgcccgtgggatcctcagacccccacccgcggctcacggccccgctgcgctccgcccccgcagagctccataccctgcggtacatccatacggcgatgacggatcccggccccgggcagccgtggtacgtggacgtggggtatgtggacggggaactcttcgtgcactacaacagcaccgcgcggaggtacgtgccccgcaccgagtggatggcggccaaggcggaccagcagtactgggatggacagacgcagatcggacagcgcaatgagcggagtgtgaaagtgagcctggacacactgcaggaacgatacaaccagaccggcggtgagcacggccggggccgcggctccgtgggtgtgggatgggctccatggcgcagtgccgcccacaccccccaggcctggccctgcccggcggcaccgtcccggggctgcccgtcacagccccaccgcgctcggggtgccgcgtcccggggggaccccaacccatccccgctgcagtgggagccccggagccggaggggcccctcaccccctgcccggctgtgtttcagggtctcacacggtgcagtggatgttcggctgtgacatcctcgaggatggcaccatccgggggtatcgtcaggtggcctacgatgggaaagacttcattgccttcgacaaagacatgaagacgttcactgcggcagttccagaggcagttcccaccaagaggaaatgggaggaaggaggtgttgctgaggggtggaagagttacctggaggaaacctgcgtggagtggctgcggagatacgtggaatacgggaaggctgagctgggcaggagaggtgagtggggtggggggggggccgcggtgtggggctggacgtggggcgggggctcagcgtggggagctcagcccggccctcattgccacctgcctgcagagcggcccgaggtgcgagtgtgggggaaggaggccgacgggatcctgaccttgtcctgccgcgctcacggcttctacccgcggcccatcgttgtcagctggctgaaggacggcgcagtgcggggccaggacgcccagtcggggggcatcatgcccaatggcgatggcacctaccacacctgggtcaccatcgatgcgcagccgggggacggggataagtaccagtgccgcgtggagcacgccagcctgccccagcccggcctctactcgtggggtgagtgaggggatgtggggctggggggctgcgggctgccccttcccctgctgatggccccgctctcccccagagccgccacagcccaacctggtgcccatcgtggcgggggtggccgtcgccattgtggccatcgccatcgtggttggtgttggattcatcatctacagacgccacgcaggtaaaagcagaggggtgcaggcgggcagtggtggcagtggggggatctgggtcccccttgggagccctcagcctggctgtgatgtgaacctgtgttgattcatctctctgtctgcagggaagaaggggaagggctacaacatcgcgcccggtgagtgatgagggcagcgctgtcccccacctctgcccagtgccagggcggtcctggggtctgcactttctcccagggtacccattcctggtgcttggggctgctccacgccccatagggagcacagggctgggtctcacagctgttcctcccttatagacagggaaggtggatccagcagctcgagcacaggtgcggtgtggggctgtgggttgggaggggtccgtgtgctctctgtggtactgcccagggctgggctatgctggggctctgcggggagacccccggagcagagggttgggatgtgaacatgggccccgtgggacaccatctcttctcatccccacagggagcaacccctccatctga

SEQ ID NO: 5 - DNA, 678 bp, Gallus gallus
atggatgaggaaattacttatgctgatttaaggcatcctacgggcagtttgcctcctgctaagcggcagcgcgttccgtgggacttccagaaagcctgcatgctgggggtgctgctgctgctgctgctgctgctggtgacagggctcagcatgtgcgtctttcagaaaacaacaccacctccaagcgctgcccggcactgccctgagaccaacgggaggaatgggacagagctctgcaagaatacctccattgagcagtacttctgccaatccaaattgagcagctctgcagcccctgctgcctgcctgctctgtccccagttctggaggcttttaggggaccgatgctatgggctttccacagaaaaggagaactggacacaagcaaaaatgaagtgtgaaaatctgcagtctcagctggctgtgctccggaagaaggcagaaaaggatcacctccagaagatggcaggtgcggagccggtgtggattggattagaagtgtccacaaatcaattgaaatgggtggacaactcctcctacaacagcacagagtttgataaccttcccgtgatggagaatggctgcgggaccttcaagaacacaaaagtggagggcgacgtctgtagtggtgaacacaagtgggtctgccagaaagaacctttacgtctccatccataa

SEQ ID NO: 6 - protein, 225 aa, Gallus gallus
MDEEITYADLRHPTGSLPPAKRQRVPWDFQKACMLGVLLLLLLLLVTGLSMCVFQKTTPPPSAARHCPETNGRNGTELCKNTSIEQYFCQSKLSSSAAPAACLLCPQFWRLLGDRCYGLSTEKENWTQAKMKCENLQSQLAVLRKKAEKDHLQKMAGAEPVWIGLEVSTNQLKWVDNSSYNSTEFDNLPVMENGCGTFKNTKVEGDVCSGEHKWVCQKEPLRLHP

SEQ ID NO: 7 - DNA, 567 bp, Gallus gallus
atggaggaagtgagggaatatccccatgttttgggcacagaatggagcaggagggaaggtcccagccgtggggcgtgcgttaccttccagctcacgatggcagcagtgttcacagtgctgctcatcactgctgttgcctttgcagtgcaggcattccagccccatccccagccctgtgctcagtgtcccttcgactggattggattcagaggaaaatgctactacttttcagaggatgagagcaattggacgagcagccagaacaactgctctgcacttggtgcttccttggctgtgtttgacagcgctgaggacttgagcttcacaatgagacacaaaggcagctccccccactgggttggcctctcccgggaaggcaaagagcatccatgggaatgggtgaaccgctctcctttgtctcacctgttccaggtgcaaggtgatggtctctgtgcatacctgggggatgccgggctcagctcctcccactgcagcgcgcggaggaattgggtttgcaccaaacccgcgttgcaaaaaccgaggaagaacttctgcatcagcacctga

SEQ ID NO: 8 - protein, 188 aa, Gallus gallus
MEEVREYPHVLGTEWSRREGPSRGACVTFQLTMAAVFTVLLITAVAFAVQAFQPHPQPCAQCPFDWIGFRGKCYYFSEDESNWTSSQNNCSALGASLAVFDSAEDLSFTMRHKGSSPHWVGLSREGKEHPWEWVNRSPLSHLFQVQGDGLCAYLGDAGLSSSHCSARRNWVCTKPALQKPRKNFCIST

SEQ ID NO: 9 - DNA, 1865 bp, Gallus gallus
tttggggcttccacttcacctgcaaaccaaaacttccacccatcagcagtgccccaaaagcagagacccataaggatatatttctgggtgctaaaactcactgcagtgacccaaatgaaaagcaattcctccctctgtggcaatgatcgcccaacagaaaacagcaaggagaagtcctagcattgccagggatagacaatgagagaggactgaaattaaatgcaatgtaatatagcggaaaaaaaaccccacaaccctcccccataaaaataagcaacaacagccacatcagtcccaatttatggaaaaaaccagtgctttcccccatgcaatcagaacaactgccactgcctttcattgttaccacctgcagttagatgcccccctccccttggaatgaaattatcccccatgactcaaagcctggctcagcaaaaagatatacccccagacccccaccccaacccattgcctgcgtccgtctgatgctgaatgcgaaaataaatgtgatgcatggtcacaaaatgggctttttcaggggggatttccacgtggatgctgcagggcagatggggaaggggtgaggggagatgcccaccagcagagttcccatgcaggacacagcagttttgctgccagcaccaggaagcagcttcccctcctttccctgctgggaaatcactcctttggaatgtttttttttttctgctgctcacccacattttgcacagggctgatcttccaggtcagcccaaactctgcatccccgcacggataacctctccctccctaagaatcagtgcatcctgcctgcctgcaaagcagctgctgagatgtcttttgcagcccttattcccgcagaccccatgcagaaccacacacatctccatcctctcctccgttggcgaggaatgggtttgcaaagggatgggcacaaccagcaatatgcaaaggaagaggtgtcgaaggtctggggagcaatgaatctgtcccccggaagatgtttccatggggcagttaaggaggagaattggaaatgaagcagatgatgcagcaatgaaactatcccagaaaagggggaaaaagcaattctggtaatgaagatacataaaggagaagggcttctcgctgtctggacgcagttctgttggttaacgtcttttctctttgtgctctttgcacttttttctttgcctgctctggtcaggatgaggcagagccctcacggggccctttcacacctttttttagcacacagaagcgcagcggccgtctcagcacccagcatggaagagaagggactgcaaataaattaaatgcgttactgaatagacagccgtaattaaaagtcaaacccatcccctcccagtattccagctgccgaggcatcggttggcacagaatcaccaaatattgcctttcttcccccatccccgcttatcagccaatgctctctgacccctaaaaggtctcgatttggggtctttttgttgttgttgttgttgttctgggtatttttaggcttttattatcagcgatttttcagcttctcactgcttaccccccagctcagcaccgcgtcgctcactgccatcgctgaacccagcggcgtttccatccctcagagagcagcaaaatgagacatcggccgtcgtgcacggaagggaaaggctgtagggtacatctacccttatttcttgggtttgtgttttgttttgttgttatttaaaagcaaaaccaagacaacaaagcccagccaatgccatttcctggcagtggacgcaggcgcaggcgggttggtcacaaagcaagaagttgctgcgggactttgtcgttttggggccgttctcgtgaacttctgagcc

SEQ ID NO: 10 - DNA, 205 bp, Gallus gallus
tgtgcaggagatggagggcaacgcagtccctccctctgatgtgtgcccaccccaaaagccccaggcgttggggtgaagctctgcaatacggctctgtgaggagtttgggctgtgcagaccccagcactgcccgggcacacggggggacatcgctgcatgcgctcatgcaatcagctcacaatcctgtgttatttctctcaataaa

SEQ ID NO: 11 - DNA, 394 bp, Gallus gallus
gcggctcccggacccgaacacgcgatgcaagaggaggaacccaaagcaaaagagctccgctttcagctgtgctcagtagcaacaggagggcggtgcgctcctccagcccaggtccgacagtgccgcctatggggctgcgcggaccgaagcaaatcccaggcggagcttcggctccaaattacatttttttgcaccgtctgactcctaatgaccgctaaaatcccaattttgggggctatccgtgcgctgcttgcaacgaccttcacccctgcgcgatgcagcagcaggtttggggggcggacggtgggaaaatatccatttttcaccggtttttctccaaagggaaatactgggaaagcaatcagcccaaaggaccctgaaatcgatgaataaa

SEQ ID NO: 12 - DNA, 792 bp, Gallus gallus
atgggggctgcgagcgggcgcggggcgctcgtggcagcgctgctgggggcggccctggggagcgtcagagccgagccatcactgcacactctgtccgaggtgctcttctgccagccggacacgccgtcgctggggctgtcagtggccttcgactcagagcagctcttctcattcgatgtccccaactcgcagtggctgccgcagctccccgatggcccctcgtggcccgcagacatcgagcagccccacgagctgctgcacgacgccgcgctgtgccgtgagctgctcgatttgctcaccagaatcgccaccgggccaaacccaatgcctgaagccaagggcatcccggtggccgatgtcttcctgcagcagcctctgcagctcggctaccccaacactctgatctgtatggtgggcaacatcttccccccagccatcactatcagctggcagcgggatggcatccccgtcaccgatggcgtcacccacctcacctacacccccaccgaggacctgggcttcatgcgcttctcctacctggcggtgacaccgcactctggtgacatctatgcctgcattgtcacccgcgagagggacaacatctctgtggtggcttactgggtgccacaggaccccatcccttcggacgtgttggccacggcggtgtgcggcgcagtgacggcgctgggcatcctgctggcactgctgggtttggggctgctgctgtccgcccgccggcgcagtatgtggggacaatggagacagcagggacacccgccccgtactcactga

SEQ ID NO: 13 - protein, 263 aa, Gallus gallus
MGAASGRGALVAALLGAALGSVRAEPSLHTLSEVLFCQPDTPSLGLSVAFDSEQLFSFDVPNSQWLPQLPDGPSWPADIEQPHELLHDAALCRELLDLLTRIATGPNPMPEAKGIPVADVFLQQPLQLGYPNTLICMVGNIFPPAITISWQRDGIPVTDGVTHLTYTPTEDLGFMRFSYLAVTPHSGDIYACIVTRERDNISVVAYWVPQDPIPSDVLATAVCGAVTALGILLALLGLGLLLSARRRSMWGQWRQQGHPPRTH

SEQ ID NO: 14 - DNA, 780 bp, Gallus gallus
atgggctttctggggtgcaacatgtgggtttgggggtgcagcatgggggttttagggatgcagtgcgccttcgtggtgcacatggccagctcctgcccactgctggccaatggctccctgggcggcttcgacctcaccgtggccttcaacaagaaccctctgctgtgctacgaccccgacgtccaccgcttctacccctgcgattgggggctgctgcacaccattgccactttgctcgccgccatcctaaatgatgataccacatgggtgcagcgtgcagaggcacgcaggcaggcgtgcactgagctggctgcacagttctggacacacacagcactgcgcaggacaccaccccaggtccgcatcgtccccatccccatctccaacgaccccgacaccgtccacctcatctgccatgtttggggcttctacccacccgcagtgaccatccagtggctgcacaacggcctcgtggtggcctcaggtgacaccaaactgctgcccaacggggactggacctacaggacacaggtggccctgagggccagcactgcagcagggagcacctacacatgctcagtgtggcactccagcctggagcagccgctgcaggaggactggagtcccaatttgtccccggcgatgatggtgaaggtggcagtggcggccatggcgctgacgttggggttggtggcactcagcgccggggttttcagcttctgtcagcggccacgggtgtggggtgtacctcctcattcatcatcttcttccccatcctga

SEQ ID NO: 15 - protein, 259 aa, Gallus gallus
MGFLGCNMWVWGCSMGVLGMQCAFVVHMASSCPLLANGSLGGFDLTVAFNKNPLLCYDPDVHRFYPCDWGLLHTIATLLAAILNDDTTWVQRAEARRQACTELAAQFWTHTALRRTPPQVRIVPIPISNDPDTVHLICHVWGFYPPAVTIQWLHNGLVVASGDTKLLPNGDWTYRTQVALRASTAAGSTYTCSVWHSSLEQPLQEDWSPNLSPAMMVKVAVAAMALTLGLVALSAGVFSFCQRPRVWGVPPHSSSSSPS

SEQ ID NO: 16 - DNA, 777 bp, Gallus gallus
atgaaactctgggggggggggacgtgggggtctctgctttatgggactgtgccttcatggtgcatgtggccaactcctgcccactggcagccaatggctctctgcgcggcttcgacctcaccgtggccttcaacaagaaccctctggtgtgctacgaccccgatggccacctcttcaacgcctgcgactgggggctgctgcacggcgtggctggacagattgccattgccctcaacaatgacagcacctgggtgcagcgtgcagaggcacggagacgggcgtgcagcaaactggctgcacagttctgggcacagacggcgctgcgcaggaccccaggtccgcatcgtccccgcacagacagggaaccccagcgtgcccatccgcctcacctgccacgtgtggggcttctacccccccgaggtgaccatcatctggctacacaatggggacatcgtggggcctggagaccactcgcccatgttcgccatccccaatgggaactggacctaccagacacaggtggccctctcggtggccccagaggtgggggacacctacacgtgctcggtgcagcatgctagcttggaggagcccctcctggaggactggcgtcctgggctgacgctggaggtgacgctgatggtggctgtggccactgtagtgatggtgttggggctcagcttgctcttcattggtgtctactgctggcgggcccaaccccctgccccaggttacgccccgcttcccggtcacaactacccttcaggcagcatctga

SEQ ID NO: 17 - protein, 258 aa, Gallus gallus
MKLWGGGTWGSLLYGTVPSWCMWPTPAHWQPMALCAASTSPWPSTRTLWCATTPMATSSTPATGGCCTAWLDRLPLPSTMTAPGCSVQRHGDGRAANWLHSSGHRRRCAGPQVRIVPAQTGNPSVPIRLTCHVWGFYPPEVTIIWLHNGDIVGPGDHSPMFAIPNGNWTYQTQVALSVAPEVGDTYTCSVQHASLEEPLLEDWRPGLTLEVTLMVAVATVVMVLGLSLLFIGVYCWRAQPPAPGYAPLPGHNYPSGSI

SEQ ID NO: 18 - DNA, 1021 bp, Gallus gallus
cagggggcatgtggtggggggagggaggggtggggctgtggggatgcccggtgttgctggggggctgctgtagggttgcatggcattgcgaggatgcagccatgaagattcacggcattgtaagtgtgcatctgtaggggcccctggcattgcaagtgtgcacctatgggagtgcccggcattgcaagggtgcacctctggacgcgtttgtcattgcaaagggtgcagctgtggtggtgaatggcattgcaagggtgtatctatgggagtgtacagcattgcaagggcgcacctatgggtgtgcctggcattgcatgcatgcacgtgtggggatgtatggcactgggggggtgcaccagtgggggtgcttgggattgcaagggtgagcctatagcagtgcctggcattgcagggttgcacgcagggatgcgtacggcattgcaggggtgcagctgccggcattgcagagggccgaacccgcccgtacggttgtgcagcgcttccagctcggagggcgcattgcagtgcggtgcattgcagtgcgaggagccactgctgcagggtgtacagtgcacgccccgaggatgtcccctcggctccaaaccccaaaaccccacgcttattaccccccaaaaacatacttttacacacaagacacatttttaccatcaaacctcacgcttttcccccaaaatccctcacaaaaacaaaatccgcgccgtgatgagacaccccagagatctacggagcctactcgtcccctgcttcattaattagagctgcttattaattgcttgcgggtggctcagcgcctattacagcgtcggggctccccgctagtttcttctatctagtaacaagtgacgcaaggtaactgcggagcgcggccattggttgagccgcacgatcatctcctgtcacagcgctggtgttccccgcagatctgttctgcctagcaaccgatgacgcgtaaagccgcgaggcacggccattggctaaactggttgccggtagcagaggg

SEQ ID NO: 19 - DNA, 390 bp, Gallus gallus
tgtcccccataagttgatccctcggtgtgggaacggtgatggtgatgtaattaaagcccttcatttgcagcgcggtgtccttgtttgtccccactccgggaagggtggcaattaatggggttgggcattgtccccatggccccaggtggcaaagtctgatcccattgcaccgcccatggggtgacgtgaggaggaggaggaggaggaggagaaggagtaggaggaggtgaaggtggcacggaggggatgaaggcagcagtgctcctgatggggccaaggggtttcagggtgctgggggcgatggggctggtgctgagctgcgggacagcaggtaggatgtggggaagtgtggggtttttggggtgaagcatgggggttttagggtgcaac

SEQ ID NO: 20 - DNA, 628 bp, Gallus gallus
tgcgggttctcactccaatcctggtccccaaaatgatcccggtccaagttctggtccccatcccagtcctggtccccattctggtcttggtcctggtcctggttctgctcctggtccctatccctgactctggtcccggtccccatcccgatgccagtcccagtcctggtccccatcctggtcctgctccttggtttggggacctcaatgactggaactcccatgtcccaacatggggacccacagtttggggtgaggggctctcaccccccaataaaaccatctgcagccccaacctcgctccaattcttcgttcccacgttgggtgggtcgggctcccagtgctcccagtgctcccagccgtctatgtcccgtaagcgtcggctccactgcattctgctccggaaacagatgacgctaccacggcgccgcctctgattggctgctccgtgccctctctccgtcccacgtccgtgaagggggggatgtggggtggagggagctgaggggccgcccccttcccccccccgctcccccctccgcgatgttggtgctattggggctgctgctgggagcgcggggggcaggtgggggtttggggttggggtgtttggggggtctctgccta

SEQ ID NO: 21 - DNA, 572 bp, Gallus gallus
tggacaccttctgtcaccaactgtccctgcgtgtccccatccctgactctgcgccgtggtgctgacattaaagacactctgcagcctctgttggtgtctctgtgggcttttggggtggggtggtgtcaccggggagaggttgggttggggtcattgcatccatgatggtgatggtgattgacattgtgcacagggagatgtccaggcgcctgtggggtctgtgttttagggccagttctgctcagtgcctccgtaagtgatctggataggtcgtcagtcatcctaattaaggaggggacaacagtgaatggggaggagccgatgactcaggctgggagtggtgatcccagaggtttcctctgctgtcagtgactccgtgctttcgctttcgcttcacaacctgagggagcgcattctgcctggcgcccgatgacgtcacataaacccccgactgccattggcggagaggcgacggaggagccaatgggggcgcggggcggggcggaggagtaggaaaagctgaaggagctgcgctgggtgcggcggacttgagagtgcagcggtgtgaggcg

SEQ ID NO: 22 - DNA, 555 bp, Gallus gallus
cgcggccccgatccccctccggcccgatcccaatgcccggcagcggccggagcttctcggaaacgagagcgtctctcattggctgaggcgatgcagcagcgacgctgctcattggccgagatggttccgcgtcatcagttgccaggcagatcggaacactgcagtttggagaggggcgggtaattgaaagtgaaagtaatggcggagcggggaggagatggagagcggcggcggtgaggggctggagggggctggagagggctggactggtaggggctggtggggtctggtggccactggtggggtctggtggttactggtggggactgtcgaggggctggagggatctgatggggactggaggggtttggtgatggctgttgtgtgctccaggctgggctgtggggagccggactgaaagtgggggccgttctaaaagcactgctgtgtgttccaggtgctgaggggagctgaggacctgcaccaggagcaccccggggagcccacctggtccagctgtgcaccagaagctctggggatccccaccccacagcc

SEQ ID NO: 23 - DNA, 1752 bp, Gallus gallus
atggggacgatgggcgcgggtcgccgcctcctcctctcgctgagcccggagcgccggcgctgcgccgcggtgatgggtctgatggcggcttcagcgctgggcgagatggccgtgccctactacatggggcgagccagcgactgggtggcccgtgaggacgagctggcagccatcctgcccatggtgctgctgggcctcagcagtgctgtcactgagctggtgtgcgatgtgaccttcgtggggacactgagccgcacgcagagccgcctccagcgccgcgtcttcgccgccgtcctgcggcagagcatcaccgagctgcgcgccgacggggccggggatgtggccatgcgggtgacgcgggatgcggaggacgtgcgcgaggctctgggtgaggcgctgagcctcctgctgtggtatctggcgcgcggcctctgcctctttgcaaccatggcctggctgtccccgcgcatggcgctgctcaccgcgctggcgctgccgctgctgctggccctgcccagggctgtggggcacttccggcaggccctggcaccacagatgcagaaggcgcaggcccgggccagcgaggtggcagtggagaccttccaggccatggccactgtgcgaagctttgccaatgaggatggggcagctgcacactaccggcagcgcctgcagcagagccaccgcctggagaagaaggatgtggccctctacactgcctctctctggaccagtggtttctcagccctggccctgaagatggggatcctctactatggggggcagctggtggccgcggggaccgtcagcactggggacctcgtcaccttcctcctctaccagatacagttcactgatgtcctgagggtcctgctcgactacttccccacactgatgaaggctgtgggctcttcggagaagatctttgagttcctggaccgggagccacaggtcgcaccctcagggacaatggcacccactgacctgcagggccacctccagctggaggatgtctggttctcctaccctgggcgccaggaacccgtcctcaagggcgtatcactggagctgcgccccggggaggtgctggcactgctgggacccccgggcgcagggaagagcactctggtggccctcgtgtcccgcctgcaccagcccacggccggacgcctgctgctggatggccaccccctccccgcctaccagcactcctacctgtgccgccaggtggccgtcgtcccccaggagccgctgctctttgcccgctcactccacgccaacatctcctatgggttggggggctgcagctgggcacaggtgacagcggccgcccgccgggtgggcgcccacgacttcatcactcgcctgccccaaggctacgacacagaggtgggcgagttgggaggacagctctccggggggcagcggcaggcggtggccattgcccgtgcgctgctgcgggacccccgcatcctcatactcgacgagcacaccagcgccctggacactgagagccagcagcaggtggagcaggagatcctcgcagccaaagggtcggggcgtgcagtgctgatggtgacggggcgggcagccctggcggcgcgggcacaacgagtggtggtgttggaggggggagaggtgcggcaggagggacccccccacgaggtgctgcgccccggcagccttctgcgggactggggacaacagggagcaccgggggagggggacagagggagtgggggggagggatag

SEQ ID NO: 24 - DNA, 2101 bp, Gallus gallus
atgccgccctacattctgcgcctgtcatgcacgctgctcctggccgacctggccctcatgttggccctggcccacttcttcccagcactggcccatttgggctgggtgggctcctggctggaggccgggctgcgcctcctggtgctggggggggccgggcagctgctggcccccaggggaccccatggggctgcggtgctgctgagcctgggccccgccatcttcctgaccctacggggctatgtaggtctgcctggagctgccccggtgctgctggccatggcaacgccgtcctggctggtgctgacccacgggacagctgtggtggcattgctcacctggagcctcctggtccccactgtggccactggggcaaaggaggcagaggcctgggtgcccctgaggcggctgctggccctcgcctggcccgagtggcccttccttggctgtgccttcctcttcctcgcattggctgcactgggtgagacctcagtgccctactgcaccgggagggctctggatgtcctccgccagggggacggcctcgccgccttcaccgctgctgtcggcctcatgtgcctggcctctgccagcagctcgctgtttgccggctgccgcggtggcctcttcaccttcatcaggttccgcttcgtcttgcgcacccgcgaccagctcttctccagcctggtgtaccgggacctcgccttcttccagaagaccacagcagctgagttggcctcccggctgaccaccgatgtgacgctggcgagcaacgtgttggcactcaatatcaacgtcatgctgaggaacctggggcaggtgctggggctctgcgccttcatgctggggctgtccccgcgcctgacaatgctggcactgctcgaagtgccgctcgccgtcaccgcacggaaagtctatgacacccggcaccagatgctgcagcgggccgtgctggatgcagcagccaacactggagcggcagtgcaggagtccatctcttccattgagatggtacgggtcttcaatggcgaggaggaggaggagtaccgctacagccaggtgctggacaggaccctacggctgcgggaccagcgggacacagagagggccatttttctcctcatccagcgggtgctgcagttggccgtgcaggcactggtgctatactgtgggcaccagcagctccgcgaggggaccctcactgccggcagcctcgtcgccttcatcctctaccagactaaagctggcagctgcgtgcaggcactggcgtactcctatggtgaccttctgagcaatgcagtggccgcctgcaaggtctttgattacctggactgggagcgagctgtgggtgctggtggcacctatgtgcccaccagactgcggggccacgtcaccttccatcgggtgtccttcgcctatcccactcgccctgagcgcctcgtcctgcaagatgtcaccttcgagctgcgccccggtgaggtgacggcgttggcggggctgaatggcagcgggaagagcacctgcgtggcactgctggagagattctatgaacctggggctggggaagtgctgctggacggggtgccgctgcgggactacgagcaccgctacctgcaccgccaggtggcactggtggggcaggaacccgtgctcttctctggctccattcgggataacattgcctacgggatggaggactgcgaagaggaggagatcatagcagctgcaaaggctgcaggtgctttgggcttcatctctgcactggagcaaggctttggcactgacgtaggggagagaggggggcagctgtcagcggggcagaagcagcgcatcgccattgcccgcgctttggtgcggcgtcccaccgtccttatcctcgacgaagccaccagtgctctggatggggacagcgatgcaatgctgcagcagtgggtgaggaatggaggggaccggacggtgctgctcatcacccaccaaccacggatgctggagaaggcagaccgcgttgtggtgctggagcatggcacggtggctgagatggggacacccgccgagctgaggacccgcggcggaccctacagccggctgctacagcacgtga

SEQ ID NO: 25 - protein, 583 aa, Gallus gallus
MGTMGAGRRLLLSLSPERRRCAAVMGLMAASALGEMAVPYYMGRASDWVAREDELAAILPMVLLGLSSAVTELVCDVTFVGTLSRTQSRLQRRVFAAVLRQSITELRADGAGDVAMRVTRDAEDVREALGEALSLLLWYLARGLCLFATMAWLSPRMALLTALALPLLLALPRAVGHFRQALAPQMQKAQARASEVAVETFQAMATVRSFANEDGAAAHYRQRLQQSHRLEKKDVALYTASLWTSGFSALALKMGILYYGGQLVAAGTVSTGDLVTFLLYQIQFTDVLRVLLDYFPTLMKAVGSSEKIFEFLDREPQVAPSGTMAPTDLQGHLQLEDVWFSYPGRQEPVLKGVSLELRPGEVLALLGPPGAGKSTLVALVSRLHQPTAGRLLLDGHPLPAYQHSYLCRQVAVVPQEPLLFARSLHANISYGLGGCSWAQVTAAARRVGAHDFITRLPQGYDTEVGELGGQLSGGQRQAVAIARALLRDPRILILDEHTSALDTESQQQVEQEILAAKGSGRAVLMVTGRAALAARAQRVVVLEGGEVRQEGPPHEVLRPGSLLRDWGQQGAPGEGDRGSGGEG

SEQ ID NO: 26 - protein, 699 aa, Gallus gallus
MPPYILRLSCTLLLADLALMLALAHFFPALAHLGWVGSWLEAGLRLLVLGGAGQLLAPRGPHGAAVLLSLGPAIFLTLRGYVGLPGAAPVLLAMATPSWLVLTHGTAVVALLTWSLLVPTVATGAKEAEAWVPLRRLLALAWPEWPFLGCAFLFLALAALGETSVPYCTGRALDVLRQGDGLAAFTAAVGLMCLASASSSLFAGCRGGLFTFIRFRFVLRTRDQLFSSLVYRDLAFFQKTTAAELASRLTTDVTLASNVLALNINVMLRNLGQVLGLCAFMLGLSPRLTMLALLEVPLAVTARKVYDTRHQMLQRAVLDAAANTGAAVQESISSIEMVRVFNGEEEEEYRYSQVLDRTLRLRDQRDTERAIFLLIQRVLQLAVQALVLYCGHQQLREGTLTAGSLVAFILYQTKAGSCVQALAYSYGDLLSNAVAACKVFDYLDWERAVGAGGTYVPTRLRGHVTFHRVSFAYPTRPERLVLQDVTFELRPGEVTALAGLNGSGKSTCVALLERFYEPGAGEVLLDGVPLRDYEHRYLHRQVALVGQEPVLFSGSIRDNIAYGMEDCEEEEIIAAAKAAGALGFISALEQGFGTDVGERGGQLSAGQKQRIAIARALVRRPTVLILDEATSALDGDSDAMLQQWVRNGGDRTVLLITHQPRMLEKADRVVVLEHGTVAEMGTPAELRTRGGPYSRLLQH

SEQ ID NO: 27 - DNA, 5000 bp, Gallus gallus
  misc_feature (1)..(5000): n is any base
ggccctcgtcccacccgatggtcctccaatgacctccatggtcatccagtgctcatcccgcggtatggccatcaccctgcagtcacccaatggtcttatcaaagttatgccatggtcatctctttgtcaccccattctcactccctgttacttctgttccctctcgttgtcccctaatggtcacaccattttctgcccgtggtcactcagcgtttctaccgttacccaatcatggtcaccccgtgttcctctccgtgatcccttcatggtcatcccatatcatctacctggacttgtgtgcggccattgggacggtcccacgccacatccttatcactaaattggagataagtgtgcaaagagtggactttggataaggaatcagttgggtggtcgcagccagagggctgtggtcaatggctgcatgtcccagtggggatggcgacaaatggggtccccggggggtccgtctgggagcggtgctcctctgcatctttgtcaaagaccaatgaagagcactgaagaccaccaaagaccactgaaaatcactgaagatcacaaagatcccccgaagatccctccatgcaaacgcagaagggtccaatatgtgctggaacctcatccttacgtcacccatggatccggggtgggtggcagtggctgtgtttaggtcggcctgtggggaaagccgggctgtcccacccatgtcccctcttccaacactgttcctgaatgagttttccctctccgaccctttttttaatgggtttcagggatttaaaattaatattgacgaagtgacggagggggtggggccacagcggagccgaaagcgaaagcagcggagagcgatggctgcggggctgcggctgctgctggcgggtgagacccgaccccccccggccccctcatgtcccaccacccatatcgccccccccccccgccctcgccccatgctgagcctctcccccacccccagggctctgctggtcccaatttagggtggaagacgccgcctcccctccgcccccccccgctccggtgcgctgcgcgctgctggagggggtggggcgcaggggagggctgccgggggggggcaatgcccgtcctgcactgctgcgctttgggggggacgcggagacccctcccgaacccggcccggagcccgaagtcaccttcaatgtcagcggtacgtggggacccccggcactgtgctgtgcgcctcctttatccccacccccctccatgtccccatctcctttacttcccacaatgctcccatcgcccccagaatgtccccagagtcccccaaacccccatgaccccccccccacgatcccctggttcccattaccctctcacgtctcccagtgtccccaagattcccattactccccgtatccccattatccccaaaatgtcccccaatgttcccatcaccccaatgttcccaaggtccctatcgctcctcaatgtcgctatgatccctattcccaaaatgtcaccaatgtccccaaaatccccattatctcccacctctccaaagtccccaagatccccattacccccaatatcctcattacaccccaaatgtccccaatgtcccctccatgtcccccagagaccccattagccccaatagctcccaaactgtccccagtgtccccattaaccccaaaatgaccccattacgccccacacccctcccaaccccatgccctcagaccccttcatccctctcactcctctctccctcgcagacccctgggggactctgaccccactcggggtccccccccggactccccccagctgcgaactgaaccccacgaacccccagaccggctctgacccatggagccgccctctgcaccccgacgcccgcagccccccaaccgcgggggggcagtggtgggtggcggcggtggggaccccgcagtacggtgtcactgcgctgctgcagggggggatgggcacagaaggaaccatcactgccgccggtaagggggaacttggggtgtccctccctgggtgtccccatgtccctatctgtccccccgtgtgtccccatttgtcccctcctctgcatgtgtcccaatgtctccatacatccctataataaccatatgtccccactcatccccatattccccatgtgtccccatatccccacacatcccagtgtgccccaacacatccccatgtgcccccccccatgcatcactaccatccccctatcccccaagtgtccctgtgtccctgcagtttctccctgtcctcatgtgttcccatgtctccatgtcactgtgtccccgtgtccccacacatcaccatgccccccatgcatccctgcgtcccccacacatccccataatccccatatttccttatctctccatgtctctgcagcgcccccatgtcccttcacctctccatgtcccccagtgtccccatatcccctcattgtccccatgccccctcacctccccgtgtcccccgtgtccctatgtccccctggtgtttccatgtcccctcatgcccccatgtcccctcatgcccccatatcccccagtgtccccatgtcccttcacctccccatgtcccccaatattcccatatcccctcacctccccatttccccccgatgttcccatgtcccctcacctccccatgtcctcacagtggccctggcggtgctcacccacaccccggccctccgggcccgtgtggggtcccccatccacctgcactgcgccttcgccgcccccccgtccccctttgtcctcgagtggcgtcaccagaacaggggtgcggggagggtcctgctggcctatgacagttccaccgcccgcgccccccgcgccacccccggggccgaactgctgctggggacacgggatggggacggggtgacagcagtgacactgcggctggcacagccatcaccgggggatgagggcacctacatctgctccgtgttcctgccccacgggcacacacagacagtgctgcagctccacgtctttggtgcgtccatgtggggcaggcggtgttcctatggggtgtggggttgggcagtgttcctatggagtgtgtatgactgggtggtattcctattggtcagataggacatatgggagcaggcggtattcctatgggggctgtagggtggatgggactgggtgatattcctgtggggggctgtagggtggatgggactgggtggtattcctatggaggctatagggtggatgggaccgggtggtattcctatgaggactataggatgaggtggcatcatcccatagttcacctgtaggtttatagggggggatgagccctataggaggagccctatgggctatatgggaccgatgtccccccacnngtctcccagagccccccaaggtgacgctgtccccgaagaacctggtggtggctccggggatgtcagcagagctacgctgccacgtgtctggcttctaccccttggatgtgacggtgacgtggcagcgccgcgccgggagctcggggacatcacagtcacccagggacacagtgatggacagctggacttcaggtcaccgccaggcagccgatggaacctacagccggacggcggcagcacggctgatccccgcacgcccccaacaccacggggacgtctacagctgcgttgtcacccacactgcactggccaaaccaatgcgcgtctccgtccgactactcctggctggtgaggggggatgtggggatattggaaacacgtggaggtattgggatgctgggaccgtggttaggagggtctgagggacatcaggaccatggcctgggacaatgggagatcatggatttgggttggggaccccacccaggatggtgacactgtgcttagggctgtcgttgtccccacaggcaccgagggaccgcacctggaggacatcacggggctcttcttggtggcctttgtcctctgtggcctcatccgttggctctaccctaaaggtgagtgctgttcccacatcccagttcccccacatcctcacaccccaatatcccaatggcccatgtccccatgagcaatgtcactatgtcccaatatcctaatgatgctgtgtacccatgtgtccccatgtctctattccactcactctttctctcccctcagctgcacgacccaaagaggaaaccaaggtaacatccccccccaaaaaccccaaacccccccaaaacacctccaagcaccccaaaactcaccattctcattncccccccnccccccatgccttgcagaaatcgcagtgacctccactccagctctcagcacctccctccactccagctctcagcacctcagctccagataaagagtttttcaccccaaagttctctctgtgtggtggtgtccccacaggtctgggtgcagaggggggagaactgggggcaaactgggagcagtgggagcagtgggaggaagtcctgggttggtgaggcagctgagtgacacctggggacatctgggtgccatcccttgtggacatctgggtgacactgcgattgcattgggtgacattgggatcccatcccaagctggacatcgggatgccatgaaggatggccacgtcccatcacatgtgggcatctgggtgacatcggggatggtcatttcccaacccatggaggcatctggatgacagtggggacacctgggtcccatccaatgtgcacatctgtgtcccatgccatatggacacctggatgacattgggaatggccaggtcccgccccactgaagacatctgggtcccactgaggaaatctgggccccttcagggacaccggggacatgattgggtaatggtgaggtcactataaagggaaccctggaggaatttgtgctgaacatgacctgatcgcagagggaaacgtctgttaaccatgaggggatcatgaaggggcagagggacagaaaggggggatatgggatgagaatagggtaa

SEQ ID NO: 28 - DNA, 1293 bp, Gallus gallus
atggctgcggggctgcggctgctgctggcggggctctgctggtcccaatttagggtggaagacgccgcctcccctccgcccccccccgctccggtgcgctgcgcgctgctggagggggtggggcgcaggggagggctgccgggggggggcaatgcccgtcctgcactgctgcgctttgggggggacgcggagacccctcccgaacccggcccggagcccgaagtcaccttcaatgtcagcgacccctgggggactctgaccccactcggggtccccccccggactccccccagctgcgaactgaaccccacgaacccccagaccggctctgacccatggagccgccctctgcaccccgacgcccgcagccccccaaccgcgggggggcagtggtgggtggcggcggtggggaccccgcagtacggtgtcactgcgctgctgcagggggggatgggcacagaaggaaccatcactgccgccgtggccctggcggtgctcacccacaccccggccctccgggcccgtgtggggtcccccatccacctgcactgcgccttcgccgcccccccgtccccctttgtcctcgagtggcgtcaccagaacaggggtgcggggagggtcctgctggcctatgacagttccaccgcccgcgccccccgcgccacccccggggccgaactgctgctggggacacgggatggggacggggtgacagcagtgacactgcggctggcacagccatcaccgggggatgagggcacctacatctgctccgtgttcctgccccacgggcacacacagacagtgctgcagctccacgtctttgagccccccaaggtgacgctgtccccgaagaacctggtggtggctccggggatgtcagcagagctacgctgccacgtgtctggcttctaccccttggatgtgacggtgacgtggcagcgccgcgccgggagctcggggacatcacagtcacccagggacacagtgatggacagctggacttcaggtcaccgccaggcagccgatggaacctacagccggacggcggcagcacggctgatccccgcacgcccccaacaccacggggacgtctacagctgcgttgtcacccacactgcactggccaaaccaatgcgcgtctccgtccgactactcctggctggcaccgagggaccgcacctggaggacatcacggggctcttcttggtggcctttgtcctctgtggcctcatccgttggctctaccctaaagctgcacgacccaaagaggaaaccaagaaatcgcagtga

SEQ ID NO: 29 - protein, 430 aa, Gallus gallus
MAAGLRLLLAGLCWSQFRVEDAASPPPPPAPVRCALLEGVGRRGGLPGGGNARPALLRFGGDAETPPEPGPEPEVTFNVSDPWGTLTPLGVPPRTPPSCELNPTNPQTGSDPWSRPLHPDARSPPTAGGQWWVAAVGTPQYGVTALLQGGMGTEGTITAAVALAVLTHTPALRARVGSPIHLHCAFAAPPSPFVLEWRHQNRGAGRVLLAYDSSTARAPRATPGAELLLGTRDGDGVTAVTLRLAQPSPGDEGTYICSVFLPHGHTQTVLQLHVFEPPKVTLSPKNLVVAPGMSAELRCHVSGFYPLDVTVTWQRRAGSSGTSQSPRDTVMDSWTSGHRQAADGTYSRTAAARLIPARPQHHGDVYSCVVTHTALAKPMRVSVRLLLAGTEGPHLEDITGLFLVAFVLCGLIRWLYPKAARPKEETKKSQ

SEQ ID NO: 30 - DNA, 487 bp, Gallus gallus
cccgtgcggacacagcccgggtgcccccccgtgctcgaagggccgggggttcccacaccgcgcccatcccctcccccgctccgcgctttcgctttcgcttcacaacctgagggagcgcattctgcctggcgcccgatgacgtcacataagactccaactaccattggcggagaggcgacggaggagccaatgggggcgcggggcggggcggaggagtaggaaaagctgaaggagctgcgctgggtgcggcggacttgagagtgcagcggtgcgaggcgatggctccgtgcgtggcgctgggcctggggctggggctgctgctcgccgccgtgtgcggggcggcggccggtgagtgcggccggaccgagacgcctccccgcccataaccccaccccggggctgtgtccgtgggatcctcagacccccacctgcggctcacggccccgctgcgctccgtccccgcagagctccataccctgcggtacat

SEQ ID NO: 31 - DNA, 801 bp, Gallus gallus
  misc_feature (1)..(802): n is any base
agttctgctcagtgcctccataagtgatctggataggtcatgagtcatcctaattaaggaggggacaacagtgaatggggaggagccgatgactcaggctgggagtggtgatcccagaggtttcctctgctgtcagtgactccgtgcctcaccgagtccgacggctccgtgcaggaaggagcgctccgcgcaccccacgcccccacccgggggggttcagcgcagccccccggccgtgctctgacagacgactgcttgccttcccattgccccccgtcgccccgacagaggccctcggcgccctcccctgggttaggaacacggccccgggggggacacagcccgagtgccctttcggtgtccgggaggcagggaggggacccccaccgcgcccgtccccgcccccgactccgcgctttcgctttcgcttcacaacctgagggagcgcattctgcctggcgcccgatgacgtctcgcgcgctcccggtcgccattggcggggcggcaacggaggaaccaatgggggcgcgggtgcgggacggggactggtcccagaacgtggaggagcggtgcggtgcggggcgatgcgcccgtgcggggcggtgggcctggggctgctgcgcctggggctgctgctcgccgccgtgtgcggggcggcggccgggtgtgcggccggaccgggacccctccccgcccgtaaccccaccccggggctgtgcccgtgggatcctcagacccacacccgcggctcacggccccgntgcgctccgtccccgcagagctccataccctgcggtacat

SEQ ID NO: 32 - DNA, 9924 bp, Gallus gallus
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcattgataccaactttgtaaaagaaaaggactggcagctgagggattgtcattccattgctggaagattgtaactcagacgctgtcaggacaagaaagagaggcctttgaaagaacattggtgggcaatttctgctgtaaagattgggcctccagattaataattgtagtagattggaaaggcatcattccagctcctaagagcgaaatattgaaaagaagactgctaataaaaagcagtctgagccctctgaagaatatctctagagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcagctgctcgaggatctgcggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatccggctgtggaatgtgtgtcagttagggtgtggaaagtccccaggctccccagcaggcagaagtatgcaaagcatgcctgcaggaattcgatatcaagcttatcgataccgtcgaattggaagagctttaaatcctggcacatctcatgtatcaatgcctcagtatgtttagaaaaacaaggggggaactgtggggtttttatgaggggttttataaaaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 33 - DNA, 35 bp, Artificial
  PCR primer
aaaaaaaagcttcttgagagtgcagcggtgcgagg

SEQ ID NO: 34 - DNA, 38 bp, Artificial
  PCR primer
aaaaaaaagcttcacagaatggcccatcattttatttc

SEQ ID NO: 35 - DNA, 7949 bp, Artificial
  pONY8.4GCMCHI
agatcttgaataataaaatgtgtgtttgtccgaaatacgcgttttgagatttctgtcgccgactaaattcatgtcgcgcgatagtggtgtttatcgccgatagagatggcgatattggaaaaattgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcgtaatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgggcactcagattctgcggtctgagtcccttctctgctgggctgaaaaggcctttgtaataaatataattctctactcagtccctgtctctagtttgtctgttcgagatcctacagttggcgcccgaacagggacctgagaggggcgcagaccctacctgttgaacctggctgatcgtaggatccccgggacagcagaggagaacttacagaagtcttctggaggtgttcctggccagaacacaggaggacaggtaagattgggagaccctttgacattggagcaaggcgctcaagaagttagagaaggtgacggtacaagggtctcagaaattaactactggtaactgtaattgggcgctaagtctagtagacttatttcattgataccaactttgtaaaagaaaaggactggcagctgagggattgtcattccattgctggaagattgtaactcagacgctgtcaggacaagaaagagaggcctttgaaagaacattggtgggcaatttctgctgtaaagattgggcctccagattaataattgtagtagattggaaaggcatcattccagctcctaagagcgaaatattgaaaagaagactgctaataaaaagcagtctgagccctctgaagaatatctctagagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgctctagaactagtggatcccccgggctgcaggagtggggaggcacgatggccgctttggtcgaggcggatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcgtttagtgaaccgtcagatcgcctggagacgccatccacgctgttttgacctccatagaagacaccgggaccgatccagcctccgcggccccaagcttcttgagagtgcagcggtgcgaggcgatgggctcgtgcggggcgctgggcctggggctgctgctcgccgccgtgtgcggggcggcggccgagctccataccctgcggtacatccgtacggcgatgacggatcccggccccgggctgccgtggttcgtggacgtggggtacgtggacggggaactcttcatgcactacaacagcaccgctcggagggctgtgccccgcaccgagtggatagcggccaacacggaccagcagtactgggacagagagacgcagatcgtacagggcagtgagcagattaaccgcgagaacctggacatactgcggcggcgctacaaccagaccggcgggtctcacacagtgcagtggatgtccggctgtgacatcctcgaggatggcaccatccgggggtatcatcaggcagcctacgatgggagagacttcgttgccttcgacaaaggcacgatgacgttaactgcggcagttccagaggcagttcccaccaagaggaaatgggaggaaggaggttatgctgaggggctgaagcagtacctggaggaaacctgcgtggagtggctgcggagatatgtggaatacgggaaggctgagctgggcaggagagagcgacccgaggtgcgagtgtgggggaaggaggccgacgggatcctgaccttgtcctgccgcgctcacggcttctacccgcggcccatcgttgtcagctggctgaaggacggcgcggtgcggggccaggacgcccagtcggggggcatcgtgcccaacggcgacggcacgtaccacacctgggtcaccatcgatgcgcagccgggggacggggacaagtaccagtgccgcgtggagcacgccagcctgccccagcccggcctctactcgtgggagccgccacagcccaacctggtgcccatcgtggcgggggtggctgtcgccattgtggccatcgccatcgtggttggtgttggattcatcatctacagacgccatgcagggaagaaggggaagggctacaacatcgcgcccgacagggaaggtggatccagcagctcgagcacagggagcaacccctccatctgagtgctgtgcttcagcctgcaaggagccaacagtccacaccagcatttggggtcggtgatggacacagccccatcctcttgacctctcagatgtccctctgcttcctatgctgactgttattctttgcctgcactgcttcctgtgaaataaaatgatgggccattctgtgaagcttatcgataccgtcgaattggaagagctttaaatcctggcacatctcatgtatcaatgcctcagtatgtttagaaaaacaaggggggaactgtggggtttttatgaggggttttataaaaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggggcactcagattctgcggtctgagtcccttctctgctgggctgaaaaggcctttgtaataaatataattctctactcagtccctgtctctagtttgtctgttcgagatcctacagagctcatgccttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccaacctggcttatcgaaattaatacgactcactatagggagaccggc

SEQ ID NO: 36 - DNA, 28 bp, Artificial
  PCR primer
cgagatcctacagttggcgcccgaacag

SEQ ID NO: 37 - DNA, 29 bp, Artificial
  PCR primer
accagtagttaatttctgagacccttgta

SEQ ID NO: 38 - DNA, 20 bp, Artificial
  PCR primer
caaggagagaaaaagcaccg

SEQ ID NO: 39 - DNA, 20 bp, Artificial
  PCR primer
gaacttcagggtcagcttgc

(19) United States
(12) Patent Application Publication
(10) Pub. No.: US 2006/0265773 A1
(43) Pub. Date: Nov. 23, 2006

(54) Methods for producing transgenic animals with modified disease resistance

(76) Inventors: Miles Carroll, Oxford (GB); James Kaufman, Compten (GB); Kyriacos Mitrophanous, Oxford (GB)

Correspondence Address: FROMMER LAWRENCE & HAUG, 745 FIFTH AVENUE- 10TH FL., NEW YORK, NY, 10151, US

(21) Appl. No.: 11/447,508

(22) Filed: Jun. 5, 2006

Related U.S. Application Data
(63) Continuation-in-part of application No. PCT/GB04/05108, filed on Dec. 3, 2004.

(30) Foreign Application Priority Data
Dec. 5, 2003 (GB) 0328248.0

(51) Int. Cl. A01K 67/027; A61K 39/00; A61K 48/00

(52) U.S. Cl. 800/19; 424/185.1; 514/44; 119/300

(57) ABSTRACT

Provided is a method or producing a transgenic animal having modified resistance to a disease. One embodiment of the method is performed by introducing a retrovirus into a cell of the animal or a cell which is capable of producing the animal, wherein the retrovirus comprises a polynucleotide sequence which encodes and is capable of expressing a protein which modifies the disease resistance of the animal, and wherein when the cell is a cell which is capable of producing the animal, producing the animal from the cell.

### CROSS REFERENCE TO RELATED APPLICATIONS

This application is a continuation-in-part of International Application Number PCT/GB2004/005108, filed Dec. 3, 2004, published as WO 2005/054280 on Jun. 16, 2005 and claiming priority to United Kingdom application number GB 0328248.0, filed Dec. 5, 2003.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to the production of transgenic animals having modified disease resistance, to animals so produced and to a method of screening for proteins capable of modified disease resistance in an animal.

### BACKGROUND OF THE INVENTION

The economic importance of disease to chicken production (both layers and broilers) is difficult to estimate as costs are not only direct (mortality and morbidity) but indirect as well (vaccinations, chemotherapy and eradication programs). Annual losses in the USA alone are estimated in the billions of dollars.

The present invention seeks to ameliorate the problems associated with chicken disease. The present invention is also applicable to ameliorating problems associated with disease in other animals, and also provides a method for investigating the immune response to disease.

### SUMMARY OF THE INVENTION

The particular haplotype of the B-F/B-L region of the chicken B locus determines life and death in response to certain infectious pathogens as well as to certain vaccines. We have found that the B-F/B-L region is much smaller and simpler than the typical mammalian MHC with the expression of a single class I molecule at a high level of RNA and protein. The peptide-binding specificity of this dominantly expressed class I molecule in different haplotypes correlates with resistance to tumours caused by Rous sarcoma virus, while the cell surface expression level correlates with susceptibility to tumours caused by Marek's disease virus. Resistance to Marek's disease is influenced by the B system haplotype of domesticated fowl, and also the Rfp-Y haplotype. A similar effect may be involved in class II β genes and response to killed viral vaccines.

The present invention allows the production of transgenic chickens, and indeed other animals, which express molecules which are associated with disease resistance to improve animal health. In more detail, the present invention also allows the production of transgenic animals which express molecules associated with the major histocompatibility complex (MHC) and which have an effect on disease resistance. The present invention also provides a method of improving an animal's response to a vaccine. The present invention further provides a method of screening for components which are associated with disease resistance by introducing such components, either singly or in combination, into such transgenic animals, or cells, and determining whether the component has any effect on disease resistance.

According to one aspect of the present invention there is provided a method of producing a transgenic animal having modified resistance to a disease comprising introducing a retrovirus into a cell of the animal, or a cell which is capable of producing the animal, wherein the retrovirus comprises a polynucleotide sequence which encodes and is capable of expressing a protein which is capable of modifying the disease resistance of the animal, and wherein when the cell is a cell which is capable of producing the animal, producing the animal from the cell.

The term “animal” is used in its broadest sense and refers to all animals including mammals, birds, fish, reptiles and amphibians.

A transgenic animal is an animal which includes in at least one of its cells a nucleotide of interest (NOI)—here the polynucleotide sequence associated with disease resistance. In one embodiment the cell is a germ line cell. In another embodiment the cell is a somatic cell. More particularly, the NOI has been introduced experimentally, e.g. using cDNA technology.

The nucleotide of interest is commonly referred to as a “transgene”, i.e. a gene that is inserted into the cell in such a way that ensures its function. When the gene is inserted into a germ line cell it should function, replicate and be transmitted as a normal gene.

The present invention encompasses chimeras and mosaics. A “chimera” is an animal composed of a mixture of genetically different cells. A “mosaic” is an animal in which the transgene is incorporated into the genome after the first cell division. The animal will be mosaic as different cells will have different sites of integration.

By “resistance” we include the ability of an animal to resist the effect of a disadvantageous substance. In other words, the natural capacity to withstand disease.

By “modify” we include the ability to affect the animal's resistance, e.g. by increasing or decreasing its natural capacity to withstand disease. It will be appreciated that by decreasing an animal's resistance to a disease, you are similarly increasing its susceptibility to a challenge. In other words, the present invention involves altering the animal's susceptibility to disease. Indeed resistance and susceptibility are opposite ends of a continuum; resistance being a measure of the ability of the host to reduce the growth, reproduction and/or disease-producing abilities of the pathogen, thus resulting in less severe symptoms of disease. In aspects of the present invention, the resistance of the animal is modified such that it is able to continue a normal production life.

As used herein “resistance” does not necessarily equate to immunity, and “susceptibility” does not necessarily equate to tolerance.

According to another aspect of the present invention there is provided a method of producing a transgenic animal cell comprising introducing a retrovirus into the cell, wherein the retrovirus comprises a polynucleotide sequence which encodes and is capable of expressing a protein which is capable of modifying the disease resistance of an animal obtainable from said cell, and optionally producing a transgenic animal from the cell.

Traditionally breeding programs for domesticated fowls are designed to breed disease resistance, as well as other commercial advantages into commercial lines. However, for example, Marek's disease still affects chickens world-wide. Virtually all commercially grown chickens are vaccinated for Marek's disease. Moreover, vaccines are only partially effective, and their effectiveness is also influenced by the B genotype and may be influenced by the Rfp-Y genotype of the birds. The present invention provides a way of improving the effectiveness of a vaccine.

According to another aspect of the present invention there is provided a method of increasing the resistance of an animal to a disease comprising introducing a retrovirus into a cell of the animal, or a cell which is capable of producing the animal, wherein the retrovirus comprises a polynucleotide sequence which encodes and is capable of expressing a protein which is capable of modifying the disease resistance of the animal, and wherein when the cell is a cell which is capable of producing the animal, producing the animal from the cell, and further comprising administering a vaccine to the animal.

In a particularly preferred aspect of the present invention, the polynucleotide sequence encodes at least one component of the major histocompatibility complex (MHC) or a component associated with the MHC, including combinations thereof. The MHC is a complex of genetic loci that encodes, amongst others, two sets of highly polymorphic cell surface molecules, termed MHC class I and MHC class II. The T-cell antigen receptor recognises processed antigen as peptide fragments bound to MHC class I or class II molecules. The MHC molecule may be expressed using its native promoter, or a variant thereof, or from a non-native promoter.

In mice the MHC is known as histocompatability-2 antigens (H-2 antigens) and in humans as human leukocyte-associated antigens (HLA antigens). The present invention encompasses MHC molecules whatever their nomenclature.

According to another aspect of the present invention, the polynucleotide sequence comprises at least one gene related to those in the MHC, such as, but not limited to, CD1 genes.

In one embodiment the MHC molecule is an MHC Class I. In another embodiment, and particularly where one is seeking improving the efficacy of a vaccine, an MHC Class II molecule is used. In another embodiment CTL vaccines, which have effector function that include class I MHC molecule/CD8 T cell responses, are used.

The polynucleotide may also encode a protein which is associated with the functioning of the MHC or is a component of the MHC. This polynucleotide encoding a protein which is associated with the functioning of the MHC may or may not form part of the MHC itself or a promoter thereof. In one embodiment the polynucleotide encodes an MHC accessory protein. MHC accessory proteins include components which are involved in the maturation process of MHC molecules, peptide loading of MHC molecules and/or the transportation of MHC molecules to the cell surface. Such accessory proteins include the transporter protein associated with antigen processing (TAP), the protein tapasin, C-type lectin receptors such as B-lec and B-NK, such as the lectin chaperones calnexin (CNX) and calrecticulin (CRT), the thiol-dependent oxidoreductase ERp57, DM and DO. In one embodiment the polynucleotide encodes at least part of the natural killer (NK) complex, and is preferably an NK receptor or its promoter.

In another embodiment, the polynucleotide sequence encodes at least one cytokine. Combinations of cytokines may also be expressed. Examples of cytokines which may be employed include IFN-alpha, IFN-beta, IFN-gamma, IL-1beta, IL-2, IL-3, IL-4, IL-6, IL-8, IL-10, IL-12beta, IL-13, IL-15, IL-18, TGF-beta4, GM-CSF.

It will be appreciated that more than one polynucleotide sequence which encodes a protein capable of modifying disease resistance may be employed. As well as different combinations within the MHC and cytokine groups, combinations of MHCs and cytokines may be used.

Preferably the retrovirus is a lentivirus. More preferably the lentivirus is HIV or EIAV. Preferably the retrovirus is pseudotyped.

In one embodiment, and particularly for human use, the retrovirus does not contain any functional accessory genes.

The polynucleotide sequence is operably linked to a constitutive, tissue-specific, spatial or inducible promoter. In a preferred embodiment the polynucleotide sequence is operably linked to its native promoter.

The retrovirus may be introduced in vivo or ex vivo.

In one embodiment the cell is in utero and may be a perinatal cell. Preferably the cell is an embryonic cell and may be a fetal cell. Preferably the cell is capable of giving rise to a germ line change and is preferably a germ cell or may be any cell involved in gametogenesis. The cell may be an oocyte, an oviduct cell, an ovarian cell, an ovum, an oogonium, a zygote, an ES cell, a blastocyte, a spermatocyte, a spermatid, a spermatozoa or a spermatogonia.

The retrovirus may be introduced into the cell via the blastoderm, umbilical cord, placenta, amniotic fluid, uterus, gonads or via intraperitoneal, intramuscular, intraspinal, intracranial, intravenous, intrarespiratory, gastrointestinal or intrahepatic administration.

In one embodiment the animal is non-human. Preferably the animal is a production animal, or a fish which is subject to aquaculture for production purposes. Thus, the animal may be a cow or a pig. The animal is preferably, and particularly for use in screening, a non-mammalian animal. In one preferred embodiment the animal is a bird, such as a domestic fowl. In a particularly preferred embodiment the animal is a chicken. In another preferred embodiment the animal is a fish.

According to another aspect of the present invention there is provided a method for producing a transgenic bird comprising introducing a retrovirus into a fertilised bird egg wherein the retrovirus comprises a polynucleotide sequence which encodes and is capable of expressing a component of the MHC, a component associated with the functioning of the MHC, a cytokine or a component related to a component of the MHC.

According to another aspect of the present invention there is provided a method for producing a transgenic fish comprises introducing a retrovirus into a fish egg wherein the retrovirus comprising a polynucleotide sequence which encodes and is capable of expressing a component of the MHC, a component associated with the functioning of the MHC, a cytokine or a component related to a component of the MHC.

Preferably the polynucleotide sequence encodes a component of the MHC or a component associated with the functioning of the MHC.

According to another aspect of the present invention there is provided a transgenic animal produced by the method of the invention. Preferably the transgenic animal is allowed to breed, and the present invention encompasses resulting offspring.

According to another aspect of the present invention there is provided a method for screening for proteins capable of modifying the resistance of an animal to disease comprising introducing a retrovirus into an animal cell wherein the retrovirus comprises a polynucleotide sequence which encodes and is capable of expressing a candidate protein, generating an animal from said animal cell and determining whether the resistance of the animal to a disease is modified.

Preferably the polynucleotide sequence encodes and is capable of expressing a component of the MHC or a component associated with the functioning of the MHC. In another embodiment the polynucleotide sequence encodes and is capable of expressing a cytokine.

### BRIEF DESCRIPTION OF THE FIGURES

FIG. 1 shows three different EIAV vectors which may be used in the present invention.

FIG. 2 shows Southern transfer analysis of genomic DNA from individual G1 birds to identify proviral insertions. Genomic DNA from G1 offspring of birds ID 1-4 (A,B) and 4-14 (C) were digested with Xba I, Bst EII or Bam HI as indicated, and hybridised with probes for lacZ (A<S) or GFP(C).

FIG. 3 shows reporter gene expression in G1 transgenic birds. (A) Western blot analysis of liver, heart, skeletal muscle, oviduct, skin, spleen, intestine, kidney, pancreas and bone marrow protein extracts from 5 adult G1 birds each containing single, independent insertions of pONY8.0cZ. 100 g of protein was loaded per lane and β-galactosidase protein detected as described in the Examples. (B) Western blot analysis of protein extracts from the transgenic birds in (A) run on the same gel for comparison of β-galactosidase protein levels between individuals. (C) Five G1 offspring of bird ID 4-14. The 4 birds on the left are transgenic for pONY8.0G and express eGFP. The bird on the right is not transgenic.

FIG. 4 shows reporter gene expression in G2 transgenic birds. (A) Western analysis of protein extracted from intestine, skin, liver and pancreas of G1 cockerels 2-2/6 and 2-2/19 and two G2 offspring of each bird. (B) Sections of pancreas, intestine and skin from a G2 offspring of 2-2/19 stained for β-galactosidase activity and comparable sections of a non-transgenic control bird. (C) Transgenic G2 embryos from G1 cockerel 2-2/19 stained for β-galactosidase activity on day 3 and day 10 of incubation.

FIG. 5 is a schematic diagram of part of the B-locus region sequence from the putative NK-receptor gene to the C4 gene in the B12 haplotype.

Sequences which may be usefully employed in preferred embodiments of the invention include:

SEQ ID NO. 1: Class I major gene (B21 haplotype, B-F2*2101 from ATG to TGA including introns)

SEQ ID NO. 2: major class I cDNA in nucleotides (B21 haplotype, B-F2*2101, from AY234769, read 5′UT and 3′UT in italics, start of mature protein (GAG) in bold)

SEQ ID NO. 3: major class I coding region in amino acids (B21 haplotype, B-F2*2101, from AY234769, read signal sequence in italics, with start of mature protein at beginning of second line)

SEQ ID NO. 4: Class 1 minor gene (B21 haplotype, B-F1*2101 from ATG to TGA including introns)

SEQ ID NO. 5: B-NK coding region (cDNA) in nucleotides (B21 haplotype from ATG to TAA)

SEQ ID NO. 6: B-NK coding region (cDNA) in amino acids (B21 haplotype, from Met to stop)

SEQ ID NO.7: B-lec coding region (cDNA) in nucleotides (B21 haplotype, from ATG to TGA)

SEQ ID NO. 8: B-lec coding region (cDNA) in amino acids (B21 haplotype, from Met to stop)

SEQ ID NO. 9: B-NK and B-lec promoter and 5′UT regions (B12 haplotype, taken from our AL023516, reads 5′ from immediately after the ATG of B-NK to immediately before the ATG of B-lec, likely not a bidirectional promoter)

SEQ ID NO. 10: B-NK 3′UT (B12 haplotype, taken from our AL023516, reads 5′ TAA of B-NK gene to polyA signal)

SEQ ID NO. 11: B-lec 3′UT (B12 haplotype, taken from our AL023516 entry, reads 5′ TGA of B-lec gene to polyA signal)

SEQ ID NO. 12: DMA coding region (cDNA) in nucleotides (B21 haplotype, from ATG to TGA)

SEQ ID NO. 13; DMA coding region (cDNA) in amino acids (B21 haplotype reads from Met to stop)

SEQ ID NO. 14: DMB1 coding region (cDNA) in nucleotides (B21 haplotype, reads from ATG to TGA)

SEQ ID NO. 15: DMB1 coding region (cDNA) in amino acids (B21 haplotype, reads from Met to stop)

SEQ ID NO. 16: DMB2 coding region (cDNA) in nucleotides (B21 haplotype, reads from ATG to TGA)

SEQ ID NO. 17: DMB2 coding region (cDNA) in amino acids (B21 haplotype, reads from Met to stop)

SEQ ID NO. 18: Promoter and 5′UT of DMA (B12 haplotype, taken from our AL023516, reads from about 1 kB upstream of DMA ATG)

SEQ ID NO. 19: 3′UT of DMA, intragenic region including promoter of DMB1 and 5′UT of DMB1 (B12 haplotype, taken from our AL023516, reads from just after TGA of DMA to just before ATG of DMB1)

SEQ ID NO. 20: 3′UT of DMB1, intragenic region including promoter of DMB2 and 5′UT of DMB2 (B12 haplotype, taken from our AL023516, reads from just after TGA of DMB1 to just before ATG of DMB2)

SEQ ID NO. 21: 3′UT of DMB2 (B12 haplotype, taken from our AL023516, reads from just after TGA of DMB2)

SEQ ID NO. 22: TAP1 and TAP2 promoter and 5′UT regions (B21 haplotype, reads 5′ from immediately after the ATG of TAP1 to immediately before the ATG of TAP2)

SEQ ID NO. 23: TAP1 coding region (cDNA) in nucleotides (B21 haplotype, reads from ATG to TAG)

SEQ ID NO. 24: TAP2 coding region (cDNA) in nucleotides (B21 haplotype, reads from ATG to TAG)

SEQ ID NO. 25: TAP1 coding region (cDNA) in amino acids (B21 haplotype, reads from Met to stop)

SEQ ID NO. 26: TAP2 coding region (cDNA) in amino acids (B21 haplotype, reads from Met to stop)

SEQ ID NO. 27: complete B21 tapasin sequence (5 Kb) including promoter, 5′UT introns and exons, and 3′UT

SEQ ID NO. 28: Tapasin coding region (cDNA) in nucleotides (B21 haplotype, from ATG to TGA)

SEQ ID NO. 29: Tapasin coding region (cDNA) in amino acids (B21 haplotype, from Met to stop)

SEQ ID NO. 30: Promoter and 5′UT for class I major gene (B21 haplotype, B-F2*2101 from line N)

SEQ ID NO. 31: Promoter and 5′UT for class 1 minor gene (B21 haplotype, B-F1*2101 from line N)

### DETAILED DESCRIPTION

Various preferred features and embodiments of the present invention will now be described by way of non-limited example.

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

The present invention can be used advantageously to obtain animals, such as birds, resistant to, or with improved immune response, to disease.

Obtaining animals with particular MHC haplotypes is valuable to, for example, breeders of domesticated fowl for the production of both individuals and flocks that are resistant to numerous diseases. However, traditionally breeding methods have not proved wholly effective and disease in chickens remains a substantial problem.

Development of a simple and efficient method for the genetic modification of chickens has proved a significant technical challenge. The earliest methods developed were based on the use of avian retroviruses, replication competent vectors derived from avian leucosis virus (ALV) and replication defective vectors derived from reticuloendotheliosis virus. The use of these vectors has generally resulted in low frequencies of germ line transmission coupled with very low levels of transgene expression, limiting their usefulness. Concerns have also been raised about the safety of these vectors for applications in biotechnology and poultry breeding, particularly the risks of generating recombinants with wild-type viruses that are widespread in commercial populations. More recently, an ALV vector has been used to express a foreign protein in egg white and accumulation at a low level demonstrated. A higher germline transduction frequency, one of 15 males, has been shown using a spleen necrosis virus-based vector and expression of the reporter gene detected in myoblasts. In contrast, the replication competent RCAS vector system, derived from Rous sarcoma virus, has proved a very useful tool for short term transduction of chick embryos in the investigation of the role of specific genes in development. Several non-viral methods for genetic modification of the avian germ line have been described, but so far the frequencies obtained are even lower than those obtained using retroviral vectors.

A new class of vectors has been developed recently, derived from members of the lentivirus class of retroviruses. These vectors have potential advantages over those derived from oncoretroviruses, including the ability to infect non-dividing cells. More significantly, from the perspective of their use in production of transgenic animals, transgenic mice have been generated efficiently using a HIV-based vector and reliable tissue-specific expression of a reporter gene was seen after germline transmission. The possible advantages of a lentiviral vector system for production of transgenic animals prompted us to test the efficiency of equine infectious anaemia virus (EIAV) vectors to transduce the chicken germ line. We found that transgenic birds could be generated at high frequency and that germ line transmission was higher than predicted from analysis of somatic cell transduction. Tissue-specific expression of reporter genes carried by these vectors was clearly demonstrated in first and second generation transgenic birds. Further information on this method may be found in our WO03/056022.

The overall aim of the present invention is to reduce animal disease, and particularly infectious disease by introducing at least one polynucleotide sequence which encodes a protein which modifies the disease resistance of an animal into an animal. The present invention also allows factors important to the function of the immune system and the animal's resistance to infections to be studied by illustrating the underlying genetic regulating mechanisms influencing the immune system. In poultry, especially infectious diseases caused by Marek's Disease Virus (MDV) and Infectious Bursal Virus (IBDV) may be treated and studied using the present invention.

Poultry, including chickens, ducks, turkeys, geese and guinea fowl, constitute a valuable source of protein in the form of both meat and eggs. However, the present invention has applicability to other production animals, including cattle, horses, goats, sheep, swine, other birds such as ostrich and emu; fish, such as salmon, trout, turbot, bass sea bream and carp; and indeed companion animals, such as dogs and cats. The present invention will thus contribute to increased management of immunity as an element of improving the health and thus obtaining a more efficient exploitation of the animals' production capacity.

The present invention may also be applicable to humans, e.g. one can envisage a method by which a cell is obtained from a human, transduced with the retrovirus and reintroduced into a patient. Similarly an in vivo approach may be envisaged.

Aspects of the present invention provide a method for producing a transgenic cell using a retroviral expression vector, and a transgenic animal which is obtainable from the transgenic cell or of which the transgenic cell forms part. More particularly, this aspect of the present invention provides a way of modifying the susceptibility of an animal to disease, and also allows the production of disease models.

MHC

MHC class I molecules are made by all nucleated cells in the body and possess a deep groove that can bind peptide epitopes, typically 8-11 amino acids long, from endogenous antigens. Endogenous antigens are proteins found within the cytosol of human cells. Examples include: viral proteins produced during viral replication, proteins produced by intracellular bacteria during their replication, proteins that have escaped into the cytosol from the phagosome of phagocytes such as antigen-presenting cells, tumour antigens, produced by cancer cells, and self peptides from cell proteins.

One of the body's major defences against viruses, intracellular bacteria, and cancers is the destruction of infected cells and tumour cells by cytotoxic T-lymphocytes or CTLs. These CTLs are effector cells derived from T8-lymphocytes during cell-mediated immunity. Both T8-lymphocytes and CTLs produce T-cell receptors (TCRs) and CD8 molecules which are anchored to their surface. The TCRs and CD8 molecules on the surface of T8-lymphocytes and CTLs are designed to recognise peptide epitopes bound to MHC class I molecules.

During the replication of viruses and intracellular bacteria within their host cell, as well as during the replication of tumour cells, viral, bacterial, or tumour proteins are degraded into a variety of peptide epitopes by cylindrical organelles called proteasomes. Other endogenous antigens such as proteins released into the cytosol from the phagosomes of antigen-presenting cells such as macrophages and dendritic cells as well as a variety of the human cell's own proteins (self-proteins) are also degraded by proteasomes. As these various endogenous antigens pass through proteasomes, proteases and peptidases chop the protein up into a series of peptides, typically 8-11 amino acids long.

A transporter protein called TAP located in the membrane of the cell's endoplasmic reticulum (ER) then transports these peptide epitopes into the endoplasmic reticulum where they bind to the grooves of various newly made MHC class I molecules. The MHC class I molecules with bound peptides are then transported to the Golgi complex and placed in exocytic vesicles. The exocytic vesicles carry the MHC class I/peptide complexes to the cytoplasmic membrane of the cell where they become anchored to its surface.

The MHC class I molecule with bound peptide on the surface of antigen-presenting cells such as macrophages and dendritic cells can be recognised by a complementary-shaped TCR/CD8 on the surface of a naïve T8-lymphocyte to initiate cell-mediated immunity.

Likewise, MHC class I molecule with bound peptide on the surface of infected cells and tumour cells can be recognised by a complementary-shaped TCR-CD8 on the surface of a cytotoxic T-lymphocyte (CTL) to initiate destruction of the cell containing the endogenous antigen. The TCR recognises the peptide while the CD8 molecule recognises the MHC class I molecule.

There are three major human MHC class I genes, designated HLA-A, HLA-B and HLA-C with multiple alleles for each, so genetically different individuals make different MHC class I molecules. Different MHC class I molecules, in turn, are capable of binding different peptides.

MHC class II molecules are made primarily by antigen-presenting cells or APCs. APCs include macrophages, dendritic cells, and B-lymphocytes. MHC class II molecules have a deep groove that can bind peptide epitopes, from 10-30 but optimally from 12-16 amino acids long, from exogenous antigens. Exogenous antigens are antigens that enter from outside the body such as bacteria, fungi, protozoa and free viruses.

These exogenous antigens enter macrophages, dendritic cells and B-lymphocytes through phagocytosis. The microbes are engulfed and placed in a phagosome. After lysosomes fuse with the phagosome, protein antigens from the microbe are degraded by proteases into a series of peptides, from 10-30 amino acids long.

Meanwhile, a variety of MHC class II molecules are being synthesised by the rough ER.

Once assembled, within the ER, a protein called the invariant chain (Ii) attaches to the peptide-binding groove of the MHC class II molecules and in this way prevents peptides designated for binding to MHC class I molecules within the ER from attaching to the MHC class II.

The MHC class II molecules with bound Ii chain are now transported to the Golgi complex, and placed in vesicles. The vesicles containing the MHC class II molecules fuse with the peptide-containing phaglysosomes. The Ii chain is removed and the peptides are now free to bind to the grooves of the MHC class II molecules. The MHC class II/peptide complexes are then transported to the cytoplasmic membrane of the APC where they become anchored to its surface.

Here the MHC class II molecules with bound peptides can be recognised by a complementary-shaped T-cell receptor (TCR) and CD4 molecule on the surface of a T4-lymphocyte.

T4-lymphocytes are T-lymphocytes displaying a surface molecule called CD4. They also have on their surface, epitope receptors called T-cell receptors (TCRs) that, in co-operation with the CD4 molecules, have a shape capable of recognising peptides from exogenous antigens bound to MHC class II molecules on the surface of APCs and B-lymphocytes. The TCR recognises the peptide while the CD4 molecule recognises the MHC class II molecule. T4-lymphocytes are cells the body uses to regulate both humoral immunity and cell-mediated immunity.

There are three major human MHC class II genes, designated HLA-DR, HLA-DQ, and HLA-DP with multiple alleles for each so genetically different individuals make different MHC class II molecules. Different MHC class II molecules, in turn, are capable of binding different peptides.

The binding on an MHC molecule can only recognise a limited number of potential antigens. The human immune system has evolved two ways to improve the range of antigens that can be recognised by the MHC. Firstly the MHC genes have been duplicated during evolution and each individual has at least two copies of each MHC gene. Secondly, MHC genes are highly polymorphic and vary between individuals.

The MHC genes are located close to each other in most vertebrates. New MHC alleles appear to have been created during evolution by recombination of segments from different MHC gene copies and this is likely to have occurred more often due to their physical closeness.

In domesticated fowl, the MHC is called the B system. This system comprises B-F, B-L and B-G genes. A second system of MHC-like genes of the chicken also exists. This system, designated Rfp-Y, consists of at least two class I genes, three class II genes and a c-type lectin gene. Further details on the chicken MHC may be found in: Kaufman J., Immunological Reviews (1995) 143:63-88; Kaufman J., et al, Nature (1999) 401:923-925; Kaufman J., et al, Immunological Reviews (1999) 167:101-117; Kaufman J., Phil. Trans. R. Soc. Lond. B. Biol. Soc. (2000) 355:1077-1084.

Experimental data indicates that resistance to Marek's disease is influenced by the haplotype of the MHC. Moreover it appears that the effectiveness of vaccines is also influenced by the genotype of the birds. Genes within B and Rfp-Y both have demonstrated influence in resistance and susceptibility to a number of diseases, including virally-induced tumours, bacterial infections and infections with protozoan parasites. There are further studies describing the influence of MHC haplotype in many poultry diseases. Since the association of MHC haplotype with disease resistance in fowl has been shown, the present invention seeks to modify the resistance/susceptibility of birds to disease. The present invention also seeks to improve the effectiveness of vaccines. It will be appreciated that the present invention may be extended to other animals.

We believe that the chicken B-F/B-L region represents a “minimum essential MHC”. The B locus contains the classical, highly expressed and polymorphic class I α and class II β multigene families, which are reduced to one or two members, with many other genes moved away or deleted from the chicken genome altogether. We have found that a single dominantly expressed class I gene determines the immune response to certain infectious pathogens, due to peptide-binding specificity and cell-surface expression level.

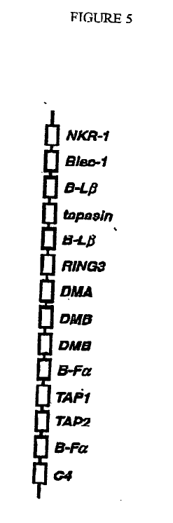

FIG. 5 shows part of the B locus region sequenced from the putative NK-receptor gene to the C4 gene in the β12 haplotype. Open boxes indicate genes. This region represents part of the B locus of the B12 haplotype, and corresponds to the B-F/B-L region by virtue of the fact it contains the dominantly expressed class I α and class IIβ genes. This 92 kb has been deposited in the EMBL database (accession no. AL023516). Four major points emerge from an analysis of the chicken sequence.

Firstly, the region is simple and compact. Secondly, some of the genes present in the MHC of typical mammals are found in this region, e.g. class I (B-F) genes and the transporter associated with processing (TAP) genes are present. Also, class II (B-L) β genes and DMA and DMB genes are present, along with the RING3 gene that encodes a nuclear kinase. One single classical class II α gene is located 5 cM away from the B locus. There is at least one gene of the class III region present, the complement component C4. Thirdly, the TAP genes are flanked by the class I (B-F) a genes, rather than being located in the class II region, and the class III region is located outside the class II region. Fourthly, the tapasin gene is present in the region and is located between two class II (B-L) β genes. Also, there are two genes containing exons encoding for C-type animal lectins. Both genes encode type II transmembrane proteins with apparent immunomodulator tyrosine based inhibitory proteins (ITIMs) in the N-terminal cytoplasmic tail, and at least one is closely related to NK receptors. In more detail, the sequencing of the entire chicken MHC also identified two potential genes encoding C-type lectin receptors, designated B-lec and B-NK. This observation was completely unexpected, as C-type lectin-like receptor genes have not been identified in the MHC of any mammal studied to date. Furthermore, isolation and analysis of the cDNA for B-lec and B-NK has shown that they share greatest homology with the C-type lectin-like Natural Killer (NK) receptors encoded in the mammalian NK Complex. Phylogenetic analysis and comparison of the genomic organisation of B-lec and B-NK sequences with other C-type lectin-like receptors provide evidence that the NK Complex and the MHC share an ancient common ancestral region. This hypothesis is supported by the presence of other C-type lectin-like receptor genes, including CD23 and DC-SIGN, in regions paralogous to the MHC in humans.

We believe that in many chicken haplotypes the genes have co-evolved over a considerable period of time, with each combination of genes showing a particular phenotype. We believe that class I (B-F) genes and the TAP genes co-evolve with important functional effects, and clearly the tapasin gene located close by may also co-evolve. We believe that the C-type animal lectin domains are indeed NK receptors that co-evolve with the class I (B-F) genes in the same haplotype, particularly with regard to the different levels of cell-surface expression, and that together this may be responsible for the very strong MHC-determined resistance and susceptibility to MDV. Finally, we believe that there are similar interactions between genes to explain the fact that, just as for class I (B-F) molecules, there is a single dominantly expressed class II molecule in many haplotypes and there is variation in cell-surface expression of class II molecules between haplotypes.

We have found that the level of total class I molecules expressed on the surface of cells varies depending on the MHC haplotype of the chicken, with the difference as much as ten-fold on certain cell types. The cell surface expression level correlates exactly with the rank order of the MHC determined susceptibility to MDV, with the highest expressor B19 the most susceptible, the lowest expressor B21 the most resistant, and the other ranged in between.

Examination of the promoter regions provides a mechanistic explanation for the difference in expression at the RNA level of the minor and major class I (B-F) genes and has identified three kinds of changes compared to the major genes. These seem to be: deletion of the enhancer A element in the B12 and B19 haplotypes; divergence in the enhancer A element in the B2, B4, B6 and B21 haplotypes; and a disruption in some part of the gene at the 5′ end in the B14 and B15 haplotypes.

Preferably the polynucleotide sequence used in the present invention is in a functional relationship with a promoter/enhancer regulatory sequences. The promoter may be any promoter, enhancer or combination known to increase expression of a gene with which it is in a functional relationship. A “functional relationship” and “operably linked” refer to a juxtaposition wherein the components described are in a relationship permitting them to function in their intended manner. For example, a regulatory sequence “operably linked” to a coding sequence is ligated in such a way that expression of the coding sequence is achieved under conditions compatible with the control sequences. The promoter/enhancer is preferably selected based on the desired expression pattern of the gene of interest and the specific promoters of known promoters/enhancers. Preferably the promoter/enhancer are the natural promoter/enhancer. However, in one aspect of the present invention these may be modified and/or changed to observe the effects of regulatory elements on the response to disease. In the present context, the term “promoter” is also used to describe a synthetic or fusion molecule, or derivative which confers, activates or enhances expression of a nucleic acid molecule in a cell.

Tapasin is a type I membrane glycoprotein which is part of a complex of molecules involved in transporting the antigen from the cytoplasm of the cell to the lumen of the endoplasmic reticulum, where the antigen can be loaded onto a class I molecule. The transporter protein called TAP, located in the membrane of a cell's ER, is involved in transporting peptide epitopes into the ER where they can bind to the grooves of MHC-I molecules. The loaded MHC class I molecule is then transported to the cell surface. TAP is an adenosine triphosphate binding cassette transporter, and is chiefly comprises of two subunits, TAP1 and TAP2. Along with TAP1/2 are calnexin, calreticulin and tapasin proteins which are associated with the bridging of the TAP complex with the MHC class I for peptide loading. Besides physically joining MHC class I molecules and TAP complexes, tapasin appears to play a role in peptide loading of empty MHC molecules. In every chicken MHC haplotype that we have examined, we found two classical class I genes that flank the TAP1 and TAP2 genes, of which one (the “minor” gene) was transcribed poorly compared with the other (the dominantly expressed of “major” gene). Interestingly, there were many more alleles of the major class I gene than the minor gene. The TAP genes are also highly polymorphic, and some of the sequence variation is consistent with differences in the specificity of translocation. In the most obvious example, we have found that the TAP1 in the B4 haplotype has positively charged residues in three positions where negatively charged residues are found in the other haplotypes examined. The peptides eluted from the total class I molecules of the B4 haplotype have three negatively charged residues, and the dominantly expressed class I molecules of the B4 haplotype has complementary positively charged residues in the binding site. We believe that the B4 TAP only pumps peptides that have three negatively charged residues into the lumen of the endoplasmic reticulum where they can bind to class I molecules. The sequence of the minor class I molecule of the B4 haplotype is incompatible with binding peptides with three negatively charged residues, so it will not be transported to the surface. Therefore, even if the minor gene was well expressed at the RNA and protein levels, it would not be involved in much antigen presentation. We have also found Tapasin to be polymorphic, ahving class I and class II B genes, in a similar manner to TAP.

It is also believed that two accessory proteins DM and DO are involved in controlling peptide loading of MHC class II molecules. It appears that DM serves as a stabilizer and chaperone for MHC class II molecules, and that DO acts as a co-chaperone for DM. Although it is thought that similar systems operate in chickens, DO has not yet been found in chickens.

The two class II (B-L) β genes are located in opposite transcriptional orientation and flank a tapasin gene, with lectin domain-containing (putative NK receptor) genes on one side and a RING3 gene followed by DM genes on the other side. All of the B haplotypes have a highly expressed (major) gene of the B-LβII family between tapasin and RING3 and a minor gene between tapasin and the lectin genes. The natural killer complex (NKC) is a genetic region in mice and humans that encodes lectin-like NK cell receptors and determines resistance to herpesvirus. It may well be therefore that the NK receptors in the MHC have an influence on resistance.

As discussed above the level of expression of class I molecules can vary due to different factors. It is also worth considering the relationship of low level expression of class I molecules to the MHC-determined resistance and the role of NK cells. At least some NK cells recognize the absence of specific class I molecules that might otherwise present antigenic peptides derived from viral antigens. Studies indicate that B21 chickens have a higher level of NK activity than other chicken strains. In this light, B21 would be regarded as an MHC that encoded class I molecules designed to elicit maximum cytotoxic NK activity, much like the levels that might be present in heterozygotes of B21 with an MHC haplotype with high expression of class I molecules. If the balance of NK and T-cell cytotoxic activity is determined in the manner, then the level of expression of particular class I molecules could represent a polymorphism that is selected by pathogens during evolution.

The polynucleotide used in the present invention may encode one or more genes which are not encoded in the MHC in mammals, but are nevertheless related to genes present in the MHC. Examples of genes related to those in the MHC are the CD1 genes. CD1 molecules are similar to MHC Class I molecules and are able to bind and present glycolipids, particularly mycobacterial membrane components.

Cytokines

Cytokines are a unique family of growth factors. Secreted primarily from leukocytes, cytokines stimulate both the humoral and cellular immune responses, as well as the activation of phagocytic cells.

All cytokines have certain properties in common. They are all small molecular weight peptides or glycopeptides. Many are produced by multiple cell types such as lymphocytes, monocytes/macrophages, mast cells, eosinophils, even endothelial cells lining blood vessels. Each individual cytokine can have multiple functions depending upon the cell that produces it and the target cell(s) upon which it acts (called pleiotropism). Also, several different cytokines can have the same biologic function (called redundancy). Cytokines can exert their effect through the bloodstream on distant target cells (endocrine), on target cells adjacent to those that produce them (paracrine) or on the same cell that produces the cytokine (autocrine). Physiologically it appears that most cytokines exert their most important effects in a paracrine and/or autocrine fashion. Their major functions appear to involve host defence or maintenance and repair of the blood elements.

Cytokines are categorised by their major specific function(s). There are four major categories of cytokines. Interferons are so named because they interfere with virus replication. There are three major types based upon the source of the interferon. Interferon alpha (IFNα) is produced by the buffy coat layer from white blood cells and is used in treatment of a variety of malignant and immune disorders. Interferon beta (IFNβ) is produced by fibroblasts and is currently being evaluated in the treatment of multiple sclerosis. Interferon gamma (IFNγ) is produced by activated T cells and is an important immunoregulatory molecule, particularly in allergic diseases.

The colony stimulating factors are so named because they support the growth and differentiation of various elements of the bone marrow. Many are named by the specific element they support such as granulocyte colony stimulating factor (G-CSF), macrophage colony stimulating factor (M-CSF), and granulocyte-macrophage colony stimulating factor (GM-CSF). Other CSFs include Interleukin (IL)-3, which can stimulate a variety of hematopoietic precursors and is being evaluated as a therapy in aplastic anemia and bone marrow transplantation; and c-Kit ligand (stem cell factor) which has recently been demonstrated as a cytokine necessary to cause the differentiation of bone marrow stem cells into their various precursor elements for eventual differentiation into RBC, WBC and megakaryocytes (platelets).

The tumour necrosis factors (TNF) are so called because injecting them into animals causes a hemorrhagic necrosis of their tumours. TNFα is produced by activated macrophages and TNFβ is produced by activated T cells (both TH and CTL). These molecules appear to be involved in the pathogenesis of septic shock and much research is aimed at trying to inhibit their activity in septic patients. Attempts have also been made to use the TNFs clinically to treat human tumors.

The largest group is the interleukins, so named because their fundamental function appears to be communication between (inter-) various populations of white blood cells (leucocytes-leukin). Interleukins (IL) are given numbers. They are produced by a variety of cell types such as monocytes/macrophages, T cells, B cells and even non-leucocytes. The major interleukins currently of greatest interest to allergists are IL-4, IL-5, IL-10 and IFNγ. IL-4 causes a switch to IgE production by differentiating B cells. IFNγ can inhibit that switch and prevent the production of specific IgE. IL-10 can actually inhibit the activity of IFNγ, allowing the original IL-4 to proceed in the IgE cascade. Thus, an allergic response can be viewed as an allergen-specific production of excess IL-4 and/or IL-10, lack of adequate IFNγ production or both. Eosinophilic inflammation, a major component of allergic reactions, is under control of IL-5 and TNFα.

It may be that there are differences between individual animals, such as chickens, in their resistance to particular diseases which are affected by differences in their cytokine genes. The present invention seeks to modify disease resistance through increasing (or decreasing) the expression of cytokines in the animal.

Examples of cytokines which are particularly useful in the present invention are IFN-alpha, IFN-beta, IFN-gamma, IL-1beta, IL-2, IL-3, IL-4, IL-6, IL-8, IL-10, IL-12beta, IL-13, IL-15, IL-18, TGF-beta4, GM-CSF.

Diseases

The present invention may be used to treat a number of diseases. By “treat” we also include prevention.

Research into the genetic composition of broiler chickens could lead to a superior breeding stock and provide economic benefits to the poultry industry world-wide. We have identified genes that affect disease resistance, thus allowing primary breeder companies to remove chickens with undesirable genes when choosing elite breeding stick. The present invention provides an approach to selecting poultry to improve broiler stock, and indeed layer stock. The present invention also allows one to identify specific genes that affect disease resistance.

Controlling disease through current vaccinations and antibiotics is becoming more difficult. The current focus is on the genetic makeup of the broiler, specifically the MHC gene complex. MHC influences a number of diseases and we have the ability to detect and analyse for those genes in broilers. We also are looking at other genes that could influence disease resistance.

As mentioned above, it has been found that poultry are susceptible to diseases such as Marek's, Newcastle disease, and IBDV. Marek's disease is caused by a lymphotrophic herpesvirus which varies widely in its ability to cause disease. Tumours can be seen after 6 weeks in unvaccinated birds and from 14 weeks in vaccinated flocks. It affects the nervous system and also produces turnouts in many of the internal organs, muscles and enlargement of nerves. Mortality in unvaccinated birds can rise to 30% or more. The tumours and skin lesions can also cause down-grading in broilers. The vaccine given to day-old chicks at the hatchery has been successful; although occasional outbreaks continue to occur due to evolution of the virus to greater virulence under the selection pressure of vaccination (known as ‘breakthrough’). The virus is classified into three serotypes. Serotype 1—all tumour causing and derived attenuated strains; Serotype 2—naturally occurring non-pathogenic strains; and Serotype 3—non-pathogenic related viruses from turkeys.

Newcastle Disease is a highly infectious Paramyxovirus disease which can affect all commercial poultry and cause heavy mortality. Causes various symptoms including respiratory and nervous disorders, diarrhoea, severe lethargy and depression and on occasions very high mortality. In young birds it causes severe reduction in growth and long term secondary disease such as colibacillosis and airsaculitis. Severe egg drops and reduced shell quality in layers. Control requires an appropriate vaccination schedule and careful vaccine administration.

Infectious Bursal disease (Gumboro) is a birnaviral infection which can cause high mortality and reduced growth rate in chicks from 2 to 6 weeks of age and occasionally up to point-of-lay. It is relavent wherever poultry are kept. The disease is of particular importance because it is immunosuppressive. Several such immunosuppresive viruses exist (eg, Chicken anemia virus). Response to subsequent vaccinations to these viruses may be less effective and other diseases more severe.

Other poultry diseases include adenovirus infection, aflatoxicosis, amyloidosis, anatipestifer infection, hexamitiasis, helminth parasites, histomoniasis, hock burns, inclusion body hepatitis, arizona infection, aspergilosis, avian clostridial infections, infectious bronchitis, infectious laryngotracheitis, infectious stunting, avian coliform infections (colibacillosis), avian encephalomyelitis (epidemic tremors), avian influenza (fowl plague), avian malaria, avian mycoplasmosis, avian rhinotracheitis (ART), keratoconjunctivitis, leucocytozoonosis, leukosis, lice, marble spleen disease, avian salmonellosis, avian staphylococcus infections, avian tuberculosis, mites, mycotoxicosis, bacterial synovitis, breast blister, bumblefoot, candidiasis, chicken anaemia, omphalitis, ornithobacterium rhinotracheale, pasteurellosis (fowl cholera), pendulous crop, chlamydiosis (ornithosis), chorioretinitis, coccidiosis, plantar pododermatitis, pseudo-tuberculosis, pullet disease, ringworm (favus), ruptured gastrocnemius tendon, colibacillosis/colisepticaemia, dermatitis, duck virus enterritiss, duck virus heptatitis, erysipelas, serpulina, spotty liver syndrome, spirocheatosis, stunting and runting syndrome, femoral head necrosis, spondylolithiasis, fleas, floppy broiler syndrome, fowl plague, swollen head syndrome, syngamus trachea, toxic fat syndrome, trichomoniasis, turkey haemorrhagic enteritis, twisted leg, vent gleet, vices, fowl pox, ganrenous dermatitis, gapes, viral tenosynovitis and yolk sac infection.

The chicken major histocompatability complex (MHC), the B complex, is a highly polymorphic group of tightly linked genes coding for cell surface antigens associated with many immunological functions. The B complex, is located on microchromosome number 16 and consists of three chromosomal regions: B^{F}, which codes for MHC class I antigens present on nearly all cell types, B^{L}, which codes for MHC class II antigens, found primarily on B cells and monocytes and B^{G} which codes for MHC class IV antigens present primarily on erythrocytes.

Variant alleles of the B complex have been shown to be linked with varying degrees of susceptibility or resistance to different parasites. For example: B21 (B²¹) allele confers resistance to Marek's Disease virus (which causes malignant lymphomas in chickens), whilst B2 (B²), B6 (B⁶) and B12 (B¹²) confer moderate resistance, in contrast to B19 (B¹⁹), B13 (B¹³) and B5 (B⁵) all of which are associated with susceptibility to Marek's Disease. B5 confers high resistance to Newcastle Disease Virus, whilst B15 confers susceptibility. B2, B21 and B13 are all linked to resistance to Infections Bursal Disease Virus (IBDV). B-F2, B-F21 and B12B¹² confer resistance to Rous Sarcoma Virus, whilst B4B⁴ and B-F24 are associated with increased susceptibility. Genotypes sharing the same BF haplotype but different BG haplotype had a similar anti tumour responses, suggesting that the BF, but not the BG region is important to tumor regression. B2 is associated with increased susceptibility to coccidiosis, but is associated with improved resistance to Staphylococcus aureus infection and Eimeria Tenella. Chickens who have B-F21B^{F21} allele show increased susceptibility to E. tenella at first, but can be protected from infection by immunization. Resistance to Pastuernella multocida (which causes Fowl Cholera) is associated with the B-G region. B5 confers resistance to Brucella whilst B12 leads to increased susceptibility. The B-F/L regions of the MHC are also linked to increased resistance to MC29 virus (an avian leukemia virus, which causes myelocytomas) with B4 and B12 homozygotes being resistant to infection by the virus and B-F but not B-G regions are involved in tumour regression.

B12 and B21 are associated with increased general antibody responsiveness (tested by injecting sheep red blood cells (SRBC), killed IBDV, Brucella Abortus and other antigens) whilst B15 and B13 are linked with decreased antibody responsiveness. Chickens homozygous for B1 alleles have low antibody responses to Salmonella Pulocrum.

Further discussion on disease associations with the chicken may be found in the following references with are hereby incorporated by reference:

B locus association with MDV resistance and susceptibility originally described:
Hansen, M. P., Van Zandt, J. N. & Law, G. R. J. (1967) Differences in susceptibility to Marek's disease in chickens carrying two different B blood group alleles. Poultry Science 46:1268.

Chicken B locus association with MDV resistance and susceptibility shown to be the MHC (and not other parts of the B complex):
Briles, W. E., Stone, H. A., and Cole, R. K., 1977. Marek's disease: effects of B histocompatibility alloalleles in resistant and susceptible chicken lines. Science 195:193-195.
Briles, W., Briles, R., Taffs, R., and Stone, H., 1983. Resistance to a malignant lymphoma in chickens is mapped to a subregion of the major histocompatibility (B) complex. Science 219:977-979.
Plachy, J., Jarajda, V, and Benda, V., 1984. Resistance to Marek's disease is controlled by a gene within the B-F region of the chicken major histocompatibility complex in Rous sarcoma regressor or progressor inbred lines of chickens. Folia Biol. (Praha) 30:251-258.

Chicken MHC association with MDV resistance and susceptibility for many haplotypes reviewed in:
Kaufman, J. (2001) The immunogeneticists perspective on protection against Marek's disease. Proceedings of the 6th International Symposium on Marek's disease, Montreal, August 2000.

Chicken MHC association with response to live MDV vaccine:
Ross N, O'Sullivan G O and Coudert F, (1996). Influence of chicken genotype on protection against Marek's disease by a herpesvirus of turkeys recombinant expressing the glycoprotein B (gB) of Marek's disease virus. Vaccine 14: 187-189

Chicken MHC association with antibody response to killed vaccines:
Bacon L D, Ismail N, Motta J V (1987) Allograft and antibody responses of 15I₅ B congenic chickens. Prog Clin Biol Res 238: 219-233
Heller E D, Uni Z, Bacon L D (1991) Serological evidence for major histocompatibility complex (B complex) antigens in broilers selected for humoral immune response. Poultry Science 70: 726-732

MHC associations with resistance and susceptibility to retroviruses:
Hlozanek, I., Corbel, C. & Dieterlen-Lievre, F. (1994) The major histocompatibility complex of chickens controls the infection of early chicken embryos by MC29 virus. Virology, 203, 29-35.
Filchev, A., Bozhkov, S., Karakoz, I. Plachy, J & Sotirov, N. (1993) Pathogenicity of avian myelocytomatosis virus MC29 in two lines of chickens. Avian Pathology, 22, 295-310.
Aeed P A, Briles W E, Zsigray R M, Collins W M. 1993. Influence of different B-complex recombinants on the outcome of Rous sarcomas in chickens. Anim Genet. 24:177-81.
Taylor R L Jr, Ewert D L, England J M, Halpern M S. 1992. Major histocompatibility (B) complex control of the growth pattern of v-src DNA-induced primary tumors. Virology 19:477-9.
Plachy J. 1984. Hierarchy of the B (MHC) haplotypes controlling resistance to rous sarcomas in a model of inbred lines of chickens. Folia Biol (Praha). 30:412-25.
Gebriel G M, Nordskog A W. 1983. Genetic linkage of subgroup C Rous sarcoma virus-induced tumour expression in chickens to the IR-GAT locus of the B complex. J Immunogenet. 1983 June; 10(3):231-5.
Bacon L D, Witter R L, Crittenden L B, Fadly A, Motta J. 1981. B-haplotype influence on Marek's disease, Rous sarcoma, and lymphoid leukosis virus-induced tumors in chickens. Poult Sci. 60:1132-9.

Chicken B locus association with RSV resistance and susceptibility shown to be the MHC (and not other parts of the B complex):
Plachy J, Jurajda V, Benda V. 1984. Resistance to Marek's disease is controlled by a gene within the B-F region of the chicken major histocompatibility complex in Rous sarcoma regressor or progressor inbred lines of chickens. Folia Biol (Praha). 30:251-8.

MHC associations with resistance and susceptibility to retroviruses reviewed in:
Plachy, J., Pink, J. R. L. & Hala, K. (1992) Biology of the chicken MHC (B complex) Critical Reviews in Immunology, 12: 47-79.

Chicken MHC association with response to other pathogens:
Clare, R. A, Taylor, R. E., Briles, W. E. and Strout, R. G. 1989. Characterization of resistance and immunity to Eimeria tenella among major histocompatibility complex B-F/B-G recombinant hosts. Poultry Science, 68: 639-645.
Cotter, P. F., Taylor, R. L. & Abplanalp, H. 1992. Differential resistance to Staphylococcus aureus challenge in major histocompatibility (B) complex congenic lines. Poultry Science, 71: 1873-1878.
Lamont S J, Bolin C, Cheville N. 1987. Genetic resistance to fowl cholera is linked to the major histocompatibility complex. Immunogenetics 25:284-9.

Diseases associated with sheep, and in some cases goats, include bluetongue, brucellosis, scrapie, contagious agalactia, contagious epididymitis, foot rot, Johne's disease, peste des petits ruminants, foot and mouth disease, pulpy kidney and sheep pox.

In cattle the MHC has been linked to variations in resistance to diseases including trypanasomaiasis, mastitis and bovine leukaemia virus. The bovine MHC also appears to influence other traits such as milk yield, growth and reproduction.

Other diseases associated with cattle include ruminant diarrhea, akabane disease, tuberculosis, anaplasmosis, anthrax, beef measles, Johne's disease, botulism, bovine ephemeral fever, pestivirus infection, foot and mouth disease, bovine spongiform encephalopathy (BSE), brucellosis, calf scours, cancer eye, cattle plague, bovine papillomatosis, coccidiosis, contagious bovine pleuro-pneumonia, fog fever, liver flukes, lumpy skin disease, bovine trichomoniasis, pinkeye, ringworm and warble fly.

Diseases associated with swine include atrophic rhinitis, pseudorabies, swine fever, ileitis, scours, foot and mouth disease, Glasser's disease, pneumonia, porcine reproductive and respiratory syndrome virus, fungal infections, sarcoptic mange, swine vesicular disease and swine exanthema.

Diseases associated with horses include African horse sickness, contagious equine metritus, dourine, epizootic lymphanitis, equine herpesvirus 1, equine infectious anaemia, equine protozoal myelitis, pleuropneumonia, viral arteritis, viral encephalitis, herpesvirus paralysis, rabies, strangles and Wobbler syndrome.

Diseases associated with fish include infectious pancreatic necrosis and associated aquatic birnaviruses, infectious haematopoietic necrosis virus, viral haemorrhagic septicaemia, infectious salmon anaemia, pancreas disease and viral erythrocytic necrosis, spring viraemia, Cyprinid herpes I infection, grass carp haemorrhagic disease, golden shine virus disease, pike fry rhabdovirus disease, viraemic iridovirus disease, rickettsial and chlamydial infections, bacterial kidney disease, Enterococcus seriolocida and Streptococcus iniae, mycobacteriosis and nocardiosis, furunculosis, infection by motile aeromonads, enteric Redmouth disease, Edwardsiella septicaemias, vibriosis, flavobacterial diseases including Columnaris disease, cold-water disease and bacterial gill disease, fungal infections such as by saprolegnia, and infection by ichthyophonus.

There may be examples of associations between MHC polymorphism and susceptibility or resistance to disease in man, including both infectious and autoimmune diseases, such as malaria, diabetes, rheumatoid arthritis and coeliac disease. It may therefore be that the difference in expression level of class I molecules and other features of the chicken MHC may also occur for individual molecules (alleles of each locus) in mammals, but is averaged out by the expression of multiple genes. If so, it may be that the same disease-resistance mechanism is operative in mammals. In this case, the minimal essential MHC of chickens may serve as a simple model system for more complicated systems in humans and other mammals.

Screen

In a further aspect the invention concerns a method to identify genes that play a role in resistance to disease.

In one embodiment cells are infected with a retrovirus which expresses a component of the MHC or a component associated with the functioning of the MHC.

In other embodiment cells are infected with a retrovirus which is capable of silencing a gene or delivering a dominant negative.

Gene silencing may be accomplished in a number of ways known to those skilled in the art using e.g. RNA, such as a short RNA, a siRNA, a short hairpin RNA or a micro-RNA capable of post-transcriptional silencing of a target gene.

Post-transcriptional gene silencing (PTGS) mediated by double-stranded dsRNA is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi). The mechanism of RNAi involves the processing of long dsRNAs into duplexes of 21-25 nucleotide (nt) RNAs. These products are called small interfering or silencing RNAs (siRNAs) which are the sequence-specific mediators of mRNA degradation. In addition to siRNAs, the expression of short RNAs may act to redirect splicing (‘exon-skipping’) or polyadenylation or to inhibit translation. It will be appreciated that other silencing mechanisms may be employed.

In the screen of the present invention, a phenotype in the resulting transgenic animal is observed and the gene that was disrupted or introduced by the retrovirus is identified as being implicated in the disease process.

MHC haplotyping can be accomplished by a variety of procedures known to those skilled in the art, including restriction fragment length polymorphism (RFLP), cDNA cloning followed by sequencing and allele-specific oligonucleotide probing.

Vaccines

Traditional attenuated vaccines for animal disease often provide incomplete protection and in some cases poorly inactivated vaccines can cause the disease they are designed to prevent. We believe that the reason some chickens die on infection with certain small pathogens is because no effective peptide derived from the pathogen is presented by the class I molecules to T cells. The same explanation may be involved in the response to vaccines that elicit a class I or class II MHC-restricted response. In mammals, such phenomena have been extensively examined as “immune response (Ir) gene effects”, but were only discernible when inbred mouse and hamster strains were immunised with molecules bearing very limited epitopes. In contrast, we find that chicken strains can show striking differences in response to complicated commercial vaccines. By introducing appropriate class II molecules into the animal it may be possible to improve efficiency of a response to a vaccine, or indeed allowing any response to the vaccine.

From the point of view of modelling, chickens represent an opportunity to understand the effects of real pathogens. For example it would be possible to examine the impact of single dominantly expressed class I and class II loci found in chickens on the epidemiology of small infectious pathogens and simple vaccines. In this sense, the minimal essential MHC of chickens may be useful as a simple model system for biomedical studies.

Retroviruses

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host cell for the purpose of replicating the vectors comprising a segment of DNA. Examples of vectors used in recombinant DNA techniques include but are not limited to plasmids, chromosomes, artificial chromosomes or viruses.

The term “expression vector” means a construct capable of in vivo or in vitro/ex vivo expression.

The retroviral vector employed in the aspects of the present invention may be derived from or may be derivable from any suitable retrovirus. A large number of different retroviruses have been identified. Examples include: murine leukemia virus (MLV), human immunodeficiency virus (HIV), human T-cell leukemia virus (HTLV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “retroviruses”, Cold Spring Harbour Laboratory Press Eds: J M Coffin, S M Hughes, H E Varmus pp 758-763.

Retroviruses may be broadly divided into two categories: namely, “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al., 1997 (ibid).

The lentiviral vector used in aspects of the present invention is capable of transducing a target non-dividing cell. One advantage of this feature is that since freshly isolated oocytes are quiescent, transduction rates may be enhanced by the use of lentiviral rather than retroviral vectors.

In a typical vector for use in the method of the present invention, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target non-dividing host cell and/or integrating its genome into a host genome.

Preferably the viral vector capable of transducing a target non-dividing or slowly dividing cell is a lentiviral vector.

Lentivirus vectors are part of a larger group of retroviral vectors. A detailed list of lentiviruses may be found in Coffin et al (“Retroviruses” 1997 Cold Spring Harbour Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells (Lewis et all 992 EMBO. J 11: 3053-3058; Lewis and Emerman 1994 J. Virol. 68: 510-516). In contrast, other retroviruses—such as MLV—are unable to infect non-dividing or slowly dividing cells such as those that make up, for example, muscle, brain, lung and liver tissue.

A “non-primate” vector, as used herein in some aspects of the present invention, refers to a vector derived from a virus which does not primarily infect primates, especially humans. Thus, non-primate virus vectors include vectors which infect non-primate mammals, such as dogs, sheep and horses, reptiles, birds and insects.

A lentiviral or lentivirus vector, as used herein, is a vector which comprises at least one component part derivable from a lentivirus. Preferably, that component part is involved in the biological mechanisms by which the vector infects cells, expresses genes or is replicated. The term “derivable” is used in its normal sense as meaning the sequence need not necessarily be obtained from a retrovirus but instead could be derived therefrom. By way of example, the sequence may be prepared synthetically or by use of recombinant DNA techniques.

The non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV). Preferably the lentivirus is an EIAV. Equine infectious anaemia virus infects all equidae resulting in plasma viremia and thrombocytopenia (Clabough, et al. 1991. J. Virol. 65:6242-51). Virus replication is thought to be controlled by the process of maturation of monocytes into macrophages.

In one embodiment the viral vector is derived from EIAV. EIAV has the simplest genomic structure of the lentiviruses and is particularly preferred for use in the present invention. In addition to the gag, pol and env genes EIAV encodes three other genes: tat, rev, and S2. Tat acts as a transcriptional activator of the viral LTR (Derse and Newbold 1993 Virology. 194:530-6; Maury, et al 1994 Virology. 200:632-42) and Rev regulates and coordinates the expression of viral genes through rev-response elements (RRE) (Martarano et al 1994 J. Virol. 68:3102-11). The mechanisms of action of these two proteins are thought to be broadly similar to the analogous mechanisms in the primate viruses (Martano et al ibid). The function of S2 is unknown. In addition, an EIAV protein, Ttm, has been identified that is encoded by the first exon of tat spliced to the env coding sequence at the start of the transmembrane protein.

In addition to protease, reverse transcriptase and integrase non-primate lentiviruses contain a fourth pol gene product which codes for a dUTPase. This may play a role in the ability of these lentiviruses to infect certain non-dividing cell types.

The viral RNA of this aspect of the invention is transcribed from a promoter, which may be of viral or non-viral origin, but which is capable of directing expression in a eukaryotic cell such as a mammalian cell. Optionally an enhancer is added, either upstream of the promoter or downstream. The RNA transcript is terminated at a polyadenylation site which may be the one provided in the lentiviral 3′ LTR or a different polyadenylation signal.

Thus the present invention employs a DNA transcription unit comprising a promoter and optionally an enhancer capable of directing expression of a non-primate lentiviral vector genome.

Transcription units as described herein comprise regions of nucleic acid containing sequences capable of being transcribed. Thus, sequences encoding mRNA, tRNA and rRNA are included within this definition. The sequences may be in the sense or antisense orientation with respect to the promoter. Antisense constructs can be used to inhibit the expression of a gene in a cell according to well-known techniques. Nucleic acids may be, for example, ribonucleic acid (RNA) or deoxyribonucleic acid (DNA) or analogues thereof. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements. Nucleic acids may comprise cDNA or genomic DNA (which may contain introns).

The basic structure of a retrovirus genome is a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. More complex retroviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a defective retroviral vector genome gag, pol and env may be absent or not functional. The R regions at both ends of the RNA are repeated sequences. U5 and U3 represent unique sequences at the 5′ and 3′ ends of the RNA genome respectively.

Preferred vectors for use in accordance with one aspect of the present invention are recombinant non-primate lentiviral vectors.

The term “recombinant lentiviral vector” (RLV) refers to a vector with sufficient retroviral genetic information to allow packaging of an RNA genome, in the presence of packaging components, into a viral particle capable of infecting a target cell. Infection of the target cell includes reverse transcription and integration into the target cell genome. The RLV carries non-viral coding sequences which are to be delivered by the vector to the target cell. An RLV is incapable of independent replication to produce infectious retroviral particles within the final target cell. Usually the RLV lacks a functional gag-pol and/or env gene and/or other genes essential for replication. The vector of the present invention may be configured as a split-intron vector. A split intron vector is described in PCT patent application WO 99/15683.

Preferably the lentiviral vector of the present invention has a minimal viral genome.

As used herein, the term “minimal viral genome” means that the viral vector has been manipulated so as to remove the non-essential elements and to retain the essential elements in order to provide the required functionality to infect, transduce and deliver a nucleotide sequence of interest to a target host cell. Further details of this strategy can be found in our WO98/17815.

A minimal lentiviral genome for use in the present invention will therefore comprise (5′) R-U5—one or more first nucleotide sequences—U3-R (3′). However, the plasmid vector used to produce the lentiviral genome within a host cell/packaging cell will also include transcriptional regulatory control sequences operably linked to the lentiviral genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example the CMV promoter. Some lentiviral genomes require additional sequences for efficient virus production. For example, in the case of HIV, rev and RRE sequence are preferably included. However the requirement for rev and RRE may be reduced or eliminated by codon optimisation. Further details of this strategy can be found in our WO01/79518.

In one embodiment of the present invention, the lentiviral vector is a self-inactivating vector.

By way of example, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al 1986 Proc Natl Acad Sci 83: 3194-3198; Dougherty and Temin 1987 Proc Natl Acad Sci 84: 1197-1201; Hawley et al 1987 Proc Natl Acad Sci 84: 2406-2410; Yee et al 1987 Proc Natl Acad Sci 91: 9564-9568). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolly et al 1983 Nucleic Acids Res 11: 1855-1872) or suppression of transcription (Emerman and Temin 1984 Cell 39: 449-467). This strategy can also be used to eliminate downstream transcription from the 3′ LTR into genomic DNA (Herman and Coffin 1987 Science 236: 845-848). This is of particular concern in human gene therapy where it is of critical importance to prevent the adventitious activation of an endogenous oncogene.

In our WO99/32646 we give details of features which may advantageously be applied to the present invention. In particular, it will be appreciated that the non-primate lentivirus genome (1) preferably comprises a deleted gag gene wherein the deletion in gag removes one or more nucleotides downstream of about nucleotide 350 or 354 of the gag coding sequence; (2) preferably has one or more accessory genes absent from the non-primate lentivirus genome; (3) preferably lacks the tat gene but includes the leader sequence between the end of the 5′ LTR and the ATG of gag; and (4) combinations of (1), (2) and (3). In a particularly preferred embodiment the lentiviral vector comprises all of features (1) and (2) and (3).

The non-primate lentiviral vector may be a targeted vector. The term “targeted vector” refers to a vector whose ability to infect/transfect/transduce a cell or to be expressed in a host and/or target cell is restricted to certain cell types within the host organism, usually cells having a common or similar phenotype.

The vector may be pseudotyped with any molecule of choice, including but not limited to envelope glycoproteins (wild type or engineered variants or chimeras) of VSV-G, rabies, Mokola, MuLV, LCMV, Sendai, Ebola.

Expression vectors may be used to replicate and express a nucleotide sequence. Expression may be controlled using control sequences, which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters.

Suitable promoting sequences are preferably strong promoters including those derived from the genomes of viruses—such as polyoma virus, adenovirus, fowlpox virus, bovine papilloma virus, avian sarcoma virus, cytomegalovirus (CMV), retrovirus and Simian Virus 40 (SV40)—or from heterologous mammalian promoters—such as the actin promoter or ribosomal protein promoter. Transcription of a gene may be increased further by inserting an enhancer sequence into the vector. Enhancers are relatively orientation and position independent, however, one may employ an enhancer from a eukaryotic cell virus—such as the SV40 enhancer on the late side of the replication origin (bp 100-270) and the CMV early promoter enhancer. The enhancer may be spliced into the vector at a position 5′ or 3′ to the promoter, but is preferably located at a site 5′ from the promoter.

Preferably, the promoter is a CMV major immediate early promoter/enhancer.

Hybrid promoters may also be used to improve inducible regulation of the expression vector.

The promoter can additionally include features to ensure or to increase expression in a suitable host. For example, the features can be conserved regions e.g. a Pribnow Box or a TATA box. The promoter may even contain other sequences to affect (such as to maintain, enhance, decrease) the levels of expression of a nucleotide sequence. Suitable other sequences include the Sh1-intron or an ADH intron. Other sequences include inducible elements—such as temperature, chemical, light or stress inducible elements. Also, suitable elements to enhance transcription or translation may be present.

The expression vector of the present invention comprises a signal sequence and an amino-terminal tag sequence operably linked to a nucleotide sequence of interest.

Delivery

The retroviral vector particles of the present invention are typically generated in a suitable producer cell. Producer cells are generally mammalian cells but can be for example insect cells. A producer cell may be a packaging cell containing the virus structural genes, normally integrated into its genome. The packaging cell is then transfected with a nucleic acid encoding the vector genome, for the production of infective, replication defective vector particles. Alternatively the producer cell may be co-transfected with nucleic acid sequences encoding the vector genome and the structural components, and/or with the nucleic acid sequences present on one or more expression vectors such as plasmids, adenovirus vectors, herpes viral vectors or any method known to deliver functional DNA into target cells.

The vectors of the invention, for example, the retroviral vectors of the first aspect of the invention, may be used to deliver an NOI to any prenatal cell. The term “prenatal” means occurring or present before birth. In one embodiment the method is applied to a cell at the embryonic stage. The term embryo includes animals in the early stages of development up to birth (or hatching). As used herein the term “embryo” includes “pre-embryo”, i.e. the structure formed after fertilisation of an ovum but before differentiation of embryonic tissue, and includes a zygote and blastocyte. The term also includes a fetal cell, i.e. an embryonic cell which is in the latter stages of development. The present invention also encompasses delivery to a perinatal cell. The term “perinatal” refers to the period from about 3 months before to about one month after birth, and includes the neonatal period. The term “neonate” refers to the first few weeks following birth.

Generally vectors of the invention, for example, the retroviral vectors of the invention may be used to deliver an NOI to any germ cell, including a primordial germ cell, or cell which is capable of giving rise to a germ line change. The term “germ cell” is the collective term for cells in the reproductive system of multicellular organisms that divide by meiosis to produce gametes. The term “gametes” refers to the haploid reproductive cells—in effect the ovum and sperm. However, as indicated above the present invention is also applicable to cells involved in gametogenesis and cells from structures in which gametogenesis take place, such as the ovary.

Gametogenesis will now be described in relation to mammals by way of example only. Vectors such as the retroviral vector may be used to deliver an NOI to any of the cells of structures mentioned below. It will be appreciated that the equivalent processes in non-mammalian organisms are also included in the present invention. In brief, gametogenesis is the process of forming gametes (by definition haploid, n) from diploid cells of the germ line. Spermatogenesis is the process of forming sperm cells by meiosis (in animals, by mitosis in plants) in specialized organs known as gonads (in males these are termed testes). After division the cells undergo differentiation to become sperm cells. Oogenesis is the process of forming an ovum (egg) by meiosis (in animals, by mitosis in the gametophyte in plants) in specialized gonads known as ovaries.

In spermatogenesis the sperm are formed from the male germ cells, spermatogonia, which line the inner wall of the seminiferous tubules in the testis. A single spermatogonium divides by mitosis to form the primary spermatocyte, each of which undergoes the initial division of meiosis to form two secondary permatocytes. Each of these then undergoes a second meiotic division to form two spermatids, which mature into spermatozoa. The testis is composed of numerous seminiferous tubules, in whose walls spermatogenesis takes place. The primordial germ cells are formed in the germinal epithelium lining towards the outside of the tubule, and as cell divisions proceed the daughter cells move towards the lumen of the tubule. All these cells are nourished and supported by neighbouring Sertoli cells.

In oogenesis a primary oocyte is formed by differentiation of an oogonium and then undergoes the first division of meiosis to form a polar body and a secondary oocyte. Following fertilisation of the egg, the secondary oocyte undergoes the second meiotic division to form the mature ovum and a second polar body. The ovary contains many follicles composed of a developing egg surrounded by an outer layer of follicle cells. After ovulation the egg moves down the oviduct to the uterus.

It will be appreciated that the vector may be administered at one locality, but the NOI is expressed or its effects felt, in another cell of the organism, i.e. the site of administration may be different from the target cell. Cells into which the vector may be administered include the examples of target cells listed above. More preferably, the cell is at the embryonic stage, and for example is in utero, the retroviral vector may be administered via the umbilical cord, placenta, or amniotic fluid, or by the intraperitoneal or intrahepatic routes. The introduction of the retroviral vector is aided by the use of ultrasound.

The production of transgenic animals, using ES cells and otherwise, is well known in the art, and described for example in Manipulating the Mouse Embryo, 2nd Ed., by B. Hogan, R. Beddington, F. Costantini, and E. Lacy. Cold Spring Harbor Laboratory Press, 1994; Transgenic Animal Technology, edited by C. Pinkert. Academic Press, Inc., 1994; Gene Targeting: A Practical Approach, edited by A. L. Joyner. Oxford University Press, 1995; Strategies in Transgenic Animal Science, edited by G. M. Monastersky and J. M. Robl. ASM Press, 1995; and Mouse Genetics: Concepts and Applications, by Lee M. Silver, Oxford University Press, 1995. A useful general textbook on this subject is Houdebine, Transgenic animals—Generation and Use (Harwood Academic, 1997)—an extensive review of the techniques used to generate transgenic animals from fish to mice and cows.

Thus, for example, the present invention permits the introduction of heterologous DNA into, for example, fertilised mammalian ova by retroviral infection. In one embodiment the fertilised egg is collected from a donor mother at the one cell stage and the transduced cell is transferred to a foster mother. Integration which occurs at the one cell stage produces an organism which is a true transgenic, i.e. transgenic throughout, including the germ cells. If integration occurs at a later stage mosaics are produced. In a highly preferred method, developing embryos are infected with a lentivirus containing the desired DNA, and transgenic animals produced from the infected embryo. Traditional transgenic methods have required that the embryonic cells are transformed ex vivo then reimplanted into the uterus. A significant advantage associated with the present invention is that the NOI can be introduced in utero. Another method which may be used to produce a transgenic animal involves introducing a nucleic acid into pro-nuclear stage eggs by retroviral infection. Injected eggs are then cultured before transfer into the oviducts of pseudopregnant recipients.

By way of a specific example for the construction of transgenic mammals, such as cows, nucleotide constructs comprising a sequence encoding a therapeutic protein are introduced using the method of the present invention into oocytes which are obtained from ovaries freshly removed from the mammal. The oocytes are aspirated from the follicles and allowed to settle before fertilisation with thawed frozen sperm capacitated with heparin and prefractionated by Percoll gradient to isolate the motile fraction.

The fertilised oocytes are centrifuged, for example, for eight minutes at 15,000 g to visualise the pronuclei for injection and then cultured from the zygote to morula or blastocyst stage in oviduct tissue-conditioned medium. This medium is prepared by using luminal tissues scraped from oviducts and diluted in culture medium. The zygotes must be placed in the culture medium within two hours following microinjection.

Oestrous is then synchronized in the intended recipient mammals, such as cattle, by administering coprostanol. Oestrous is produced within two days and the embryos are transferred to the recipients 5-7 days after estrous. Successful transfer can be evaluated in the offspring by Southern blot.

Alternatively, the desired constructs can be introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric strains which exclusively comprise the ES progeny can be obtained using conventional cross-breeding. This technique is described, for example, in WO91/10741.

In one embodiment a transgenic bird is produced by a method comprising infecting a bird egg with the recombinant retroviral particle. Preferably infecting the bird egg comprises contacting the embryonic blastodisc of the bird egg with the retrovirus. In more detail, transgenic birds are generated by delivering a recombinant retrovirus to the primordial germ cells of early stage avian embryos. In one embodiment, freshly laid eggs are obtained and placed in a temperature controlled, humidified incubator. Preferably the embryonic blastodisc in the egg is gradually rotated to lie on top of the yolk. This may be accomplished by any method known in the art, such as by rocking the egg regularly. The recombinant retrovirus is subsequently delivered into the space between the embryonic disk and the perivitelline membrane; although the viral solution may be delivered by any known method. In a preferred embodiment a window is opened in the shell, the virus is injected through the window and the shell window is closed. The eggs are preferably incubated until hatching. Hatched chicks are preferably raised to sexual maturity and mated.

In another embodiment a transgenic fish is produced by a method that comprises infecting a fish egg with the recombinant retroviral particles. Infecting the fish egg preferably comprises delivering the retrovirus to the space between the chorion and the cell membrane of the fish egg. In more detail, transgenic fish are created by delivering the recombinant retrovirus to single cell fish embryos. Fertilised fish eggs are collected by any method know in the art. The recombinant retrovirus is then preferably delivered to the space between the chorion and the cell membrane. This may be achieved by loading a solution of the recombinant retrovirus into a pipette. The pipette may then be used to pierce the chorion membrane and deliver the viral suspension. Injected embryos are preferably returned to a temperature-controlled water tank and allowed to mature. At sexual maturity the founder fish are preferably mated.

Analysis of animals which may contain transgenic sequences would typically be performed by either PCR or Southern blot analysis following standard methods. If desired, the organism can be bred to homozygosity.

Compositions

The present invention also provides a pharmaceutical composition for treating an animal, wherein the composition comprises a therapeutically effective amount of the delivery system of the present invention. Since the delivery system is a viral delivery system then the composition may in addition or in the alternative comprise a viral particle produced by or obtained from same. The pharmaceutical composition may be for human or animal usage. Typically, a physician will determine the actual dosage which will be most suitable for an individual subject and it will vary with the age, weight and response of the particular individual.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The delivery of one or more therapeutic genes by a delivery system according to the invention may be used alone or in combination with other treatments or components of the treatment.

Various preferred features and embodiments of the present invention will now be further described with reference to the following Examples:

### EXAMPLES

### Example 1

### EIAV Vectors and Preparation of Virus Stocks

The vectors pONY8.0cZ and pONY8.0G have been described previously (1). The vector pONY8.4GCZ, previously described in WO 03/064665, has a number of modifications including alteration of all ATG sequences in the gag-derived region to ATTG, to allow expression of eGFP downstream of the 5′LTR. The 3′ U3 region has been modified to include the Moloney leukaemia virus U3 region. Vector stocks were generated by FuGENE6 (Roche), transfection of human kidney 293T cells plated on 10 cm dishes with 2 μg of vector plasmid, 2 μg of gag/pol plasmid (pONY3.1) and 1 μg of VSV-G plasmid (pRV67) (1). 36-48 hours after transfection supernatantants were filtered (0.22 μm) and stored at −70° C. Concentrated vector preparations were made by initial low speed centrifugation at 6,000 g for 16 hours at 4° C. followed by ultracenrifugation at Xg for 90 minutes at 4° C. The virus was resuspended in formulation buffer for 2-4 hours (1), aliquoted and stored at −70° C. Transduction was carried out in the presence of 8 μg/ml polybrene.

### Example 2

### Production and Analysis of Transgenic Birds

Approximately 1-2 μl of viral suspension was microinjected into the sub-germinal cavity beneath the blastodermal embryo of new-laid eggs. Embryos were incubated to hatch using phases II and III of the surrogate shell ex vivo culture system (2). DNA was extracted from the CAM of embryos that died in culture at or after more than twelve days of development using Puregene genomic DNA purification kit (Flowgen, Asby de la Zouche, U.K.). Genomic DNA samples were obtained from CAM of chicks at hatch, blood samples from older birds and semen from mature cockerels. PCR analysis was carried out on 50 ng DNA samples for the presence of proviral sequence. To estimate copy number control PCR reactions were carried out in parallel on 50 ng aliquots of chicken genomic DNA with vector plasmid DNA added in quantities equivalent to that of a single copy gene (1×), a 10-fold dilution (0.1×) and a 100-fold dilution (0.01×) as described previously (3). Primers used: pONY8.0cZ and pONY8.4GCZ+5′CGAGATCCTACAGTTGGCGCCCGAACAG and −5′ACCAGTAGTTAATTTCTGAGACCCTTGTA-3′; pONY8.0G+5′CAAGGAGAGAAAAAGCACCG and −5′GAACTTCAGGGTCAGCTTGC-3′. The number of proviral insertions in individual G₁ birds was analysed by Southern transfer. Genomic DNA extracted from whole blood was digested with BamHI, BstEII or XbaI. Digested DNA was resolved on a 0.6% (w/v) agarose gel then transferred to nylon membrane (Hybond-N, Amersham Phramacia Biotech, Amersham U.K.). Membranes were hybridised with ³²P-labelled probes for the reporter gene lacZ or eGFP at 65° C. Hybridisation was detected by autoradiography. All experiments, animal breeding and care procedures were carried out under license from the U.K. Home Office.

### Example 3

### Production of Founder Transgenic Birds and Germline Transmission

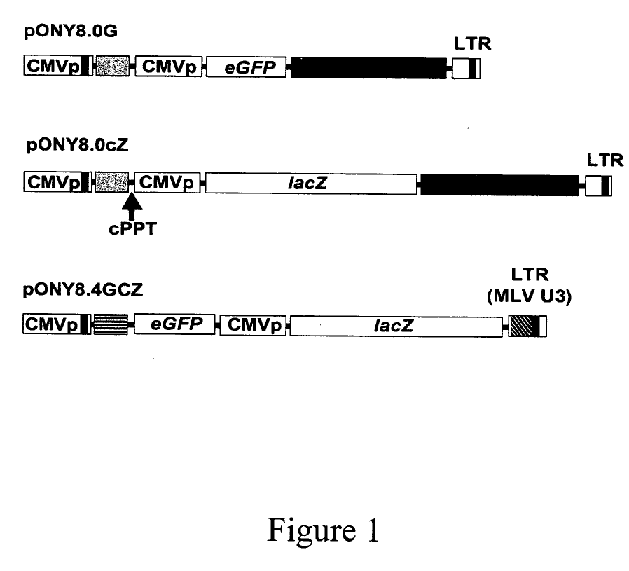

Three different EIAV vectors (FIG. 1) were used and packaged virus produced pseudotyped with vesicular stomatitis virus glyco-protein (VSV-G). These vectors have been used to transducer a number of tissues, both in vitro and in vivo (1,4-6). The vector preparations were concentrated to give titres of approximately 10⁸ to 10⁹ transducing units per millilitre (T.U./ml). One to two microlitres of concentrated virus was injected into the subgerminal cavity below the developing embryonic disc of new-laid eggs, which were then cultured to hatch. Genomic DNA was extracted from chorioallantoic membrane (CAM) of any chicks that survived past the midpoint of incubation (10 days), including hatched chicks, and analysed by PCR to detect specific vector sequences (embryos injected with pONY8.0cZ and pONY8.4ZCG only, Table 1). The PCR analysis was used to estimate the copy number of the vector sequence with respect to the amount of chicken genomic DNA present, to a sensitivity in the order of one copy per hundred cells. In experiment 1, the pONY8.cZ virus stock was diluted 10-fold, to allow comparison of injection of a particular vector at different concentrations. Ten embryos and chicks that developed after injection of 1-1.25×10⁵ T.U. of pONY8.0cZ were analysed for vector sequences and 7 (70%) contained vector sequence at a level of 0.01-0.05 copies per genome in the CAM. When the concentrated virus was injected (experiment 2), 1-1.25×10⁶ T.U./embryo, a higher proportion of embryos and chicks contained vector sequences: of 11 embryos and chicks analysed 9 (82%) contained vector sequences at a level of 0.01 to 0.5 copies per genome. 7.2×10⁵-2.8×10⁶ T.U. of the second vector, pONY8.4GCZ, was injected per embryo and 9 embryos and chicks analysed. Four of these contained vector sequences at an estimated level of 0.01-0.05 copies per genome. The vector pONY8.cZ transduced the chick embryo cells more efficiently than pONY8.4GCZ, possibly due to the presence of the cPPT sequence (1). The third vector, pONY8.0G, was produced at a higher concentration, 9.9×10⁹ T.U./ml, and of 17 embryos injected, only one survived to hatch, suggesting that injection of such a high number of infectious viral particles reduced survival. The surviving chick (bird ID 4-14) was screened as above and found to contain vector sequence at a level equivalent to one copy per cell.

In the experiments summarised in Table 1 a total of 19 chicks hatched, a hatch rate of 34% of injected embryos. These G₀ birds were raised to sexual maturity and semen samples from males screened for presence of the vector sequences. The results for individual birds are shown in Table 2. Nine of the eleven cockerels produced were crossed to stock hens and their G₁ offspring screened to identify transgenic birds and therefore germline transmission of the transgenes (Table 2). All the cockerels transmitted the vector to a proportion of their offspring, with frequencies ranging from 4 to 29%. The bird (ID 4-14) hatched after injection of pONY8.0G, a cockerel, was also crossed to stock hens and 45% (20/44) of the G₁ chicks identified as transgenic.

The frequency of germline transmission was very close to the frequency predicted from the PCR analysis of DNA from semen but, in every case, higher than predicted from analysis from DNA samples from CAM taken at hatch. Blood samples were taken from several cockerels and PCR analysis closely matched the results from the CAM DNA analysis (data not shown). The results suggest that the germ line had been transduced at an approximately 10-fold higher frequency than the somatic tissues.

### Example 4

### Analysis of G₁ Transgenic Birds and Transmission to G₂

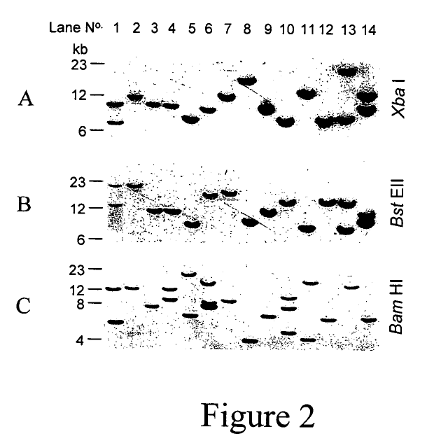

The founder transgenic birds were transduced at a stage of development when embryos consist of an estimated 60,000 cells, approximately 50 of which are thought to give rise to primordial germ cells (7,8). We expected the G₁ birds to result from separate transduction events of individual primordial germ cells and therefore that different birds would have independent provirus insertions, representing transduction of single germ cell precursors. It was also possible that individual cells would have more than one proviral insertion. Four G₀ cockerels, transduced with pONY8.0cZ, were selected for further analysis of their transgenic offspring (Table 3). Genomic DNA from individual G₁ birds was analysed by Southern blot. Samples were digested separately with Xba I and Bst EII, restriction enzymes that cut once within the integrated EIAV provirus, and hybridised with a vector-specific probe. This enabled estimation of the number of proviral insertions in each G₁ bird and of the number of different insertions present in the offspring of each G₀. An example of this analysis is shown in FIG. 2a,b and the results summarised in Table 3. The majority of G₁s carried single proviral insertions but several contained multiple copies, with a maximum of 4 detected in one bird. Some offspring of each G₀ bird carried the same proviral insertion, indicating that they were derived from the same germ cell precursor. The offspring of bird 4-14, transduced with pONY8.0G, were also analysed by Southern blot and found to contain between 1 and 4 provirus insertions (FIG. 2c).

Three male G₁ offspring of bird 2-2: 2-2/6, 16 and 19, were crossed to stock hens to analyse transmission frequency to the G₂ generation. Cockerels 2-2/6 and 2-2/19 had single proviral insertions and the ratios of transgenic to non-transgenic offspring, 14/30 (47%) and 21/50 (42%), did not differ significantly from the expected Mendelian ratio. Cockerel 2-2/16 had two proviral insertions and 79% (27/34) of the G₂ offspring were transgenic, reflecting the independent transmission of two insertions.

### Example 5

### Transgene Expression in G₁ and G₂ Transgenic Embryos and Birds

The vectors pONY8.0cZ and pONY8.4GCZ carried the reporter gene lacZ under control of the human CMV immediate early enhancer/promoter (CMVp) and pONY8.0G carried the reporter eGFP, also controlled by CMVp. Expression of lacZ was analysed by staining to detect β-galactosidase activity and by western analysis of protein extracts from selected tissues, to identify β-galactosidase protein. This analysis was carried out on G₁ and G₂ embryos and birds carrying pONY8.0cZ insertions. Expression of eGFP was analysed using UV illumination.

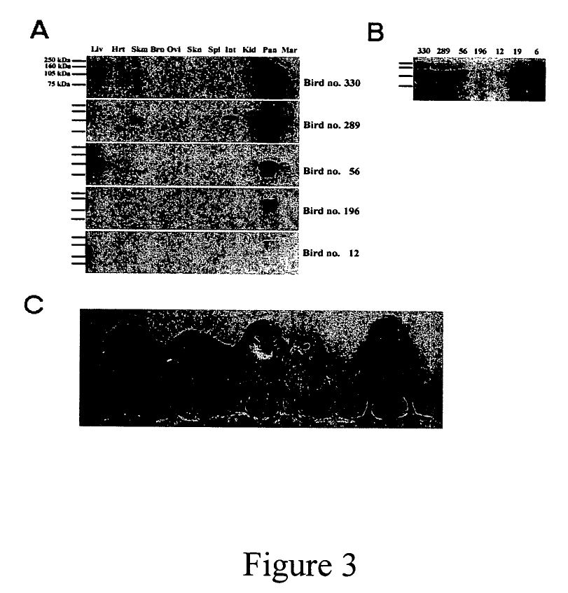

Protein extracts were made from a range of tissues from 5 pONY8.0cZ G₁ birds, each containing a different single provirus insertion, and analysed on a western blot, to detect β-galactosidase protein (FIG. 3A). A protein of the expected 110 kDa was detected in some tissues from the transgenic birds. Expression was consistently high in pancreas, and lower levels of protein present in several other tissues, including liver, intestine and skin. The protein extracts from the pancreas of each bird were run on a single gel for direct comparison (FIG. 3B). The pattern of protein expression was consistent between the individual birds but the overall amounts of protein varied. This tissue-specific pattern of expression is very similar to the pattern of expression seen in transgenic mice containing a CMVp-lacZ transgene (9). The overall level of transgene expression probably varied between individuals due to effects of the site of integration of each provirus. Expression of the GFP reporter gene in the G₀ cockerel 4-14, carrying the vector pONY8.0G, was examined under UV light. GFP fluorescence in the skin and beak was easily detected and was obvious in all 16 G₁ transgenic chicks (FIG. 3C), confirming the observation that the CMVp is active in the skin in transgenic birds. Expression was also detected in the pancreas (see supplementary information).

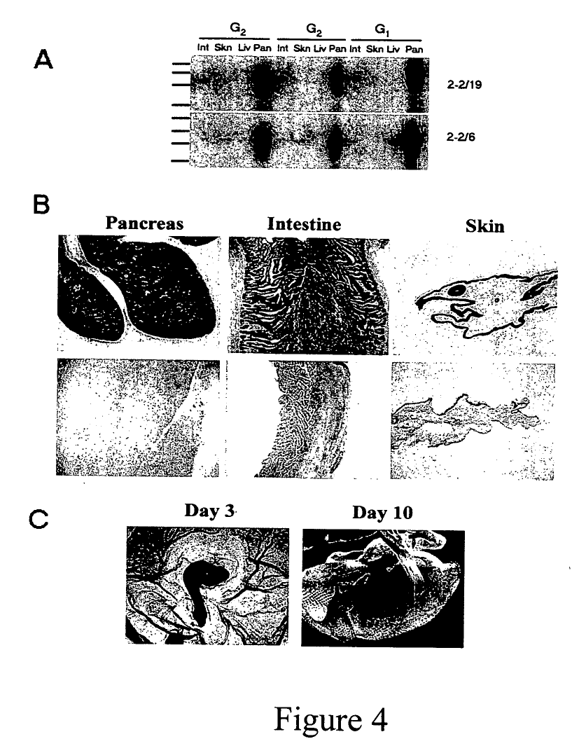

To establish if transgene expression was maintained after germ line transmission, western analysis was carried out on tissue extracts from two G₁ cockerels, 2-2/6 and 2-2/19, that each had a single proviral insertion, and two G₂ offspring from each cockerel (FIG. 4A). β-galactosidase protein levels are very similar in the parent and two offspring and the pattern of expression, predominantly in the pancreas, is also very similar. Tissue sections from these birds were also analysed (FIG. 4B). High levels of β-galactosidase activity were seen in all the cells of the pancreas, in the villi of the intestine and in the epidermis and feather follicles in sections of the skin. Expression of lacZ in embryos was also investigated by staining intact embryos at 3 and 10 days of incubation. Expression of the lacZ was most obvious in the extraembryonic region of 3 day embryos and in the skin, particularly the feather follicles, at 10 days. The pattern of expression seen in G₁ (data not shown) and G₂ embryos (FIG. 4C) was similar.

We have shown that we can obtain a very high frequency of germline transgenic birds, stable transmission from one generation to the next, and a tissue-specific pattern of transgene expression that is also maintained after germline transmission. These results indicate that the use of retroviral vectors will overcome many of the problems encountered so far in development of a robust method for production of transgenic birds expressing the polynucleotides used in the present invention.

### Example 6

### Transgene Expression in G₁ and G₂ Transgenic Embryos and Birds

The relevant EAIV based vectors to deliver the genes of interest can be constructed using standard molecular biology techniques. Below can be found an example of the cloning of MHCI cDNA (SEQ ID NO. 2) into an EIAV vector genome (pONY8.4GCZ, SEQ ID NO.32) allowing for efficient gene transfer and expression in the target cells/animal.

Using the primers shown in SEQ ID NO. 33 and SEQ ID NO. 34, PCR can be used to generate the sequence required with Hind III sites flanking it. The PCR product can be digested with Hind III and inserted into pONY8.4GCZ cut with the same enzymes, replacing the Lac Z ORF with that of MHCI cDNA. This results in plasmid pONY8.4GCMCHI (SEQ ID NO. 35) which may be used in either transient transfections to generate viral vector (as described previously in Azzouz et al.) or used to make stable producers.

The invention is further described by the following numbered paragraphs:

1. A method of producing a transgenic animal having modified resistance to a disease comprising introducing a retrovirus into a cell of the animal, or a cell which is capable of producing the animal, wherein the retrovirus comprises a polynucleotide sequence which encodes and is capable of expressing a protein which modifies the disease resistance of the animal, and wherein when the cell is a cell which is capable of producing the animal, producing the animal from the cell.

2. A method of producing a transgenic animal cell comprising introducing a retrovirus into the cell, wherein the retrovirus comprises a polynucleotide sequence which encodes and is capable of expressing a protein which modifies the disease resistance of an animal obtainable from said cell, and optionally producing a transgenic animal from the cell.

3. A method of increasing the resistance of an animal to a disease comprising introducing a retrovirus into a cell of the animal or a cell which is capable of producing the animal, wherein the retrovirus comprises a polynucleotide sequence which encodes and is capable of expressing a protein which modifies the disease resistance of the animal, and wherein when the cell is a cell which is capable of producing the animal, producing the animal from the cell, and further comprising administering a vaccine to the animal.

4. A method according to any preceding paragraph wherein the polynucleotide sequence encodes at least one component of the major histocompatibility complex (MHC) or a component associated with the MHC.

5. A method according to paragraph 4 wherein the component is an MHC molecule

6. A method according to paragraph 5 wherein the MHC molecule is an MHC Class I or MHC Class II molecule.

7. A method according to paragraph 4 wherein the MHC component is a MHC molecule promoter.

8. A method according to paragraph 4 wherein the MHC component is an MHC accessory protein or a promoter thereof.

9. A method according to paragraph 8 wherein the MHC accessory protein is selected from TAP, tapasin, a C-type lectin receptor, calnexin, calrecticulin, Erp57, DM and DO, or a promoter thereof.

10. A method according to paragraph 4 wherein the MHC component is a natural killer (NK) receptor or a promoter thereof.

11. A method according to any one of paragraphs 1 to 3 wherein the polynucleotide sequence encodes a cytokine.

12. A method according to any one of paragraphs 1 to 3 wherein the polynucleotide sequence encodes a component related to a component of the MHC.

13. A method according to paragraph 12 wherein the component is a CD1 molecule.

14. A method according to paragraph 11 wherein the cytokine is selected from IFN-alpha, IFN-beta, IFN-gamma, IL-1beta, IL-2, IL-3, IL-4, IL-6, IL-8, IL-10, IL-12beta, IL-13, IL-15, IL-18, TGF-beta4, GMCSF.

15. A method according to any preceding paragraph wherein the retrovirus is a lentivirus.

16. A method according to paragraph 15 wherein the lentivirus is HIV or EIAV.

17. A method according to any preceding paragraph wherein the retrovirus is pseudotyped.

18. A method according to any preceding paragraph wherein the retrovirus does not contain any functional accessory genes.

19. A method according to any preceding paragraph wherein the polynucleotide sequence is operably linked to a constitutive, tissue-specific, spatial or inducible promoter.

20. A method according to any preceding paragraph wherein the retrovirus is introduced in vivo or ex vivo.

21. A method according to paragraph 20 wherein the cell is in utero.

22. A method according to paragraph 21 wherein the cell is a perinatal cell.

23. A method according to paragraph 22 wherein the cell is an embryonic cell.

24. A method according to paragraph 23 wherein the cell is a fetal cell.

25. A method according to any preceding paragraph wherein the cell is capable of giving rise to a germ line change.

26. A method according to paragraph 25 wherein the cell is a germ cell.

27. A method according to paragraph 25 wherein the cell is involved in gametogenesis.

28. A method according to any one of paragraphs 25 to 27 wherein the cell is an oocyte, an oviduct cell, an ovarian cell, an avum, an oogonium, a zygote, an ES cell, a blastocyte, a spermatocyte, a spermatid, a spermatozoa or a spermatogonia.

29. A method according to any preceding paragraph wherein the retrovirus is introduced into the cell via the blastoderm, umbilical cord, placenta, amniotic fluid, uterus, gonads or via intraperitoneal, intramuscular, intraspinal, intracranial, intravenous, intrarespiratory, gastrointestinal or intrahepatic administration.

30. A method according to any preceding paragraph wherein the animal is non-human.

31. A method according to paragraph 30 wherein the animal is a cow or a pig.

32. A method according paragraph 30 wherein the animal is a non-mammalian animal.

33. A method according to paragraph 32 wherein the animal is a domestic fowl.

34. A method according to paragraph 33 wherein the animal is a chicken.

35. A method according to paragraph 32 wherein the animal is a fish.

36. A method for producing a transgenic bird comprising introducing a retrovirus into a fertilised bird egg wherein the retrovirus comprises a polynucleotide sequence which encodes and is capable of expressing a component of the MHC, a component associated with the MHC, a cytokine or a component related to a component of the MHC.

37. A method for producing a transgenic fish comprising introducing a retrovirus into a fish egg wherein the retrovirus comprises a polynucleotide sequence encodes and is capable of expressing a component of the MHC, a component associated with the MHC, a cytokine or a component related to a component of the MHC.

38. A transgenic animal produced by the method of any preceding paragraph.

39. A transgenic animal produced by allowing the animal of paragraph 38 to breed.

40. A method for screening for proteins capable of modifying the resistance of an animal to disease comprising introducing a retrovirus into an animal cell wherein the retrovirus comprises a polynucleotide sequence which encodes and is capable of expressing a candidate protein, generating an animal from said animal cell and determining whether the resistance of the animal to a disease is modified.

41. A method according to paragraph 40 wherein the polynucleotide sequence encodes and is capable of expressing a component of the MHC, a component associated with the MHC, or a cytokine.

42. A method according to paragraph 40 wherein the polynucleotide sequence encodes and is capable of expressing a component related to a component of the MHC.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

TABLE 1
PCR analysis of embryos and chicks after injection of two EIAV vectors

| Experiment | | No. eggs | | Genome equivalents | | | No. |
|---|---|---|---|---|---|---|---|
| number | Vector | injected | 0 | <0.01-0.05 | 0.1-0.5 | 1 | hatched |
| 1 | PONY8.0cZ | 19 | 3 | 7 | 0 | 0 | 5 |
| | Titre: 1-1.25 × 10⁵ | | | | | | |
| 2 | PONY8.0cZ | 17 | 2 | 5 | 4 | 0 | 8 |
| | Titre: 1-1.25 × 10⁶ | | | | | | |
| 3 | PONY8.4GCZ | 20 | 5 | 4 | 0 | 0 | 6 |
| | Titre: 7.2 × 10⁵-2.8 × 10⁶ | | | | | | |
| Total | | 56 | 10 | 16 | 4 | 0 | 19 |

TABLE 2
PCR analysis of hatched chicks and germ line transmission from founder
cockerels

| | | | Germline | | | Genome |
|---|---|---|---|---|---|---|
| Males | Genome equivalents | | transmission | | Females | equivalents |
| Bird ID | CAM | Semen | Transgenic/total | | Bird ID | CAM |
| 1-1 | 0 | 0.05 | 1/14 | (7%) | 1-2 | 0.01 |
| 1-4 | 0.01 | 0.5 | 16/55 | (29%) | 1-3 | 0 |
| 1-5 | 0.01 | 0.1 | Nd | | | |
| 2-2 | 0.1 | 1 | 4/20 | (20%) | 2-1 | 0.1 |
| 2-3 | 0 | <0.01 | Nd | | 2-5 | 0 |
| 2-4 | 0.1 | 0.5 | 19/67 | (28%) | 2-6 | 0.05 |
| 2-8 | 0.05 | 0.5 | 15/60 | (25%) | 2-7 | 0.05 |
| 3-1 | 0 | 0.05 | 1/25 | (4%) | 3-3 | 0.01 |
| 3-2 | 0 | 0.05 | 3/64 | (5%) | 3-6 | <0.01 |
| 3-4 | <0.01 | 0.05 | 4/100 | (4%) | | |
| 3-5 | <0.01 | 0.1 | 9/82 | (11%) | | |

TABLE 3
Estimation of number of provirus insertions in the genome of G₁ birds

| | | Number of birds | | | | |
|---|---|---|---|---|---|---|
| | Total G₁ | with N insertions | | | | Total no. |
| Bird ID | Analysed | 1 | 2 | 3 | 4 | independent insertions |
| 1-4 | 14 | 11 | 3 | 0 | 0 | 10 |
| 2-2 | 4 | 3 | 1 | 0 | 0 | 4 |
| 2-4 | 14 | 14 | 11 | 2 | 1 | 14 |
| 2-8 | 14 | 10 | 1 | 2 | 1 | 19 |

### REFERENCES

(1) Rholl, J. B. et al. Design, production, safety, evaluation and clinical applications of nonprimate lentiviral vectors. Methods Enzymol. 346, 466-500 (2002).
(2) Perry, M. M. A complete culture system for the chick embryo. Nature 331, 70-72 (1988).
(3) Sherman et al. Transposition of the Drosophila element mariner into the chicken germ line. Nature Biotech. 16, 1050-1053 (1998).
(4) Corcoran et al. Retinoic acid receptor β2 and neurite outgrowth in the adult mouse spinal cord in vitro. J. Cell Sci. 115, 3779-3786 (2002).
(5) Azzouz, M. et al. Multicistronic lentiviral vector-mediated striatal gene transfer of aromatic L-amino acid decarboxylase, tyrosine, hydrolase and GTP cyclohydrolase I induces sustained transgene expression, dopamine production and functional improvement in a rat model of Parkinson's disease. J. Neurosci. 22, 10302-10312 (2002).
(6) Bienemann, A. S. et al. Long-term replacement of a mutated non-functional CNS gene: reversal of hypothalamic diabetes insipidus using an EIAV-based lentiviral vector expressing arginine vasopressin. Mol. Ther. 7, 588-596 (2003).
(7) Ginsburg, M. & Eyal-Giladi, H. Primordial germ cells of the young chick blastoderm originate from the central zone of the A. pellucida irrespective of the embryo-forming process. Development 101, 209-211 (1987).
(8) Karagenc, L. Cinnamon, Y., Ginsburg, M. & Petitte, J. Origin of primordial germ cells in the prestreak chick embryo. Dev. Genet. 19, 290-301 (1996).

(9) Zhan, Y., Brady, J. L., Johnston, A. M. & Lew, A. M. Predominant transgene expression in exocrine pancreas directed by the CMV promoter. DNA Cell Biol 19, 639-645 (2000).

```
SEQ ID NO. 1: Class I major gene (B21 haplotype, B-F2*2101 from ATG to TGA
including introns)
ATGGGCTCGT GCGGGGCGCT GGGCCTGGGG CTGCTGCTCG CCGCCGTGTG
CGGGGCGGCG GCCGGTGAGT GCGGCCGGAC CGGGACCCCT CCCCGCCCAT
AACCCCACCC CGGGGCTGTG TCCGTGGGAT CCTCAGACCC CCACCCGCGG
CTCACGGCCC CGCTGCGCTC CGTCCCCGCA GAGCTCCATA CCCTGCGGTA
CATCCGTACG GCGATGACGG ATCCCGGCCC CGGGCTGCCG TGGTTCGTGG
ACGTGGGGTA CGTGGACGGG GAACTCTTCA TGCACTACAA CAGCACCGCT
CGGAGGGCTG TGCCCCGCAC CGAGTGGATA GCGGCCAACA CGGACCAGCA
GTACTGGGAC AGAGAGACGC AGATCGTACA GGGCAGTGAG CAGATTAACC
GCGAGAACCT GGACATACTG CGGCGGCGCT ACAACCAGAC CGGCGGTGAG
CACGGCCGGG GCCGCGGCTC CGTGGGTGTG GGATGGGCTC CATGGCGCAG
TGCCGCCCAC ACCCCCCAGG CCTGGCCCTG CCCGGCGGCA CCGTCCCGGG
GCTGCCCGTC ACAGCCCCAC CGCGCTCGGG GTGCCGCGTC CCGGGGGGAC
CCCAACCCAT CCCCGCTGCA GTGGGAGCCC CGGAGCCGGA GGGGCCCCTC
ACCCCCTGCC CGGCTGTGTT TCAGGGTCTC ACACAGTGCA GTGGATGTCC
GGCTGTGACA TCCTCGAGGA TGGCACCATC CGGGGGTATC ATCAGGCAGC
CTACGATGGG AGAGACTTCG TTGCCTTCGA CAAAGGCACG ATGACGTTAA
CTGCGGCAGT TCCAGAGGCA GTTCCCACCA AGAGGAAATG GGAGGAAGGA
GGTTATGCTG AGGGGCTGAA GCAGTACCTG GAGGAAACCT GCGTGGAGTG
GCTGCGGAGA TATGTGGAAT ACGGGAAGGC TGAGCTGGGC AGGAGAGGTG
AGCGGAGTGG GGGGGGCCGC AGTGTGGGGC TGGACGTGGG GCGGGGGCTG
AGCGTGGGGA GCTCAGCCCG GCCCTCACTG CCGCCCGCCC GCAGAGCGAC
CCGAGGTGCG AGTGTGGGGG AAGGAGGCCG ACGGGATCCT GACCTTGTCC
TGCCGCGCTC ACGGCTTCTA CCCGCGGCCC ATCGTTGTCA GCTGGCTGAA
GGACGGCGCG GTGCGGGGCC AGGACGCCCA GTCGGGGGGC ATCGTGCCCA
ACGGCGACGG CACGTACCAC ACCTGGGTCA CCATCGATGC GCAGCCGGGG
GACGGGGACA AGTACCAGTG CCGCGTGGAG CACGCCAGCC TGCCCCAGCC
CGGCCTCTAC TCGTGGGGTG AGTGAGGGGA TGTGGGGCTG GGGGGCTGCG
GGCTGCCCCT TCCCCTGCTG ATGGCCCCGC TCTCCCCCAG AGCCGCCACA
GCCCAACCTG GTGCCCATCG TGGCGGGGGT GGCTGTCGCC ATTGTGGCCA
TCGCCATCGT GGTTGGTGTT GGATTCATCA TCTACAGACG CCATGCAGGT
AAAAGCAGAG GGGTGCAGGC GGGCCGTGGG GGCAGTGGGG GCATCTGGGT
CCCCCTAGGG AGCCCTCAAC CTGGCTGTGA TGTGAACCTG TGTTGATTCA
TCTCTCTGTC TGCAGGGAAG AAGGGGAAGG GCTACAACAT CGCGCCCGGT
GAGTGATGAG GGCAGCGCTG TCCCCCACCT CTGCCCAGTG CCCGGGCGGT
CCTGGGGTCT CCACTTTCTC TCAGGGTACC CATTCCTGGT GCTTGGGGCT
GCTCCATGCC CCATAGGGAG CACAGGACTG GGTCTCACAG CTGTTCCTCC
CTTATAGACA GGGAAGGTGG ATCCAGCAGC TCGAGCACAG GTGCGGTGTG
GGGCTGTGGG TTGGGAGGGG TCCATGTGCT CTCGGTGGTA CTGCCCAGGG
CTGGGCTATG CTGGGACTCT GCGGGGAGAC CCCCGGAGCA GAGGGTTGGG
ATGTGAACCT GGCCCCGTGG GACATCATCC CTTCTCATCC CCACAGGGAG
CAACCCCTCC ATCTGA
SEQ ID NO. 2: major class I cDNA in nucleotides (B21 haplotype, B-F2*2101,
from AY234769, read 5′UT and 3′UT in italics, start of mature protein (GAG)
in bold)
cttgagagtg cagcggtgcg aggcgatggg ctcgtgcggg gcgctgggcc tggggctgct
gctcgccgcc gtgtgcgggg cggcggccga gctccatacc ctgcggtaca tccgtacggc
gatgacggat cccggccccg ggctgccgtg gttcgtggac gtggggtacg tggacgggga
actcttcatg cactacaaca gcaccgctcg gagggctgtg ccccgcaccg agtggatagc
ggccaacacg gaccagcagt actgggacag agagacgcag atcgtacagg gcagtgagca
gattaaccgc gagaacctgg acatactgcg gcggcgctac aaccagaccg gcgggtctca
cacagtgcag tggatgtccg gctgtgacat cctcgaggat ggcaccatcc gggggtatca
tcaggcagcc tacgatggga gagacttcgt tgccttcgac aaaggcacga tgacgttaac
tgcggcagtt ccagaggcag ttcccaccaa gaggaaatgg gaggaaggag gttatgctga
ggggctgaag cagtacctgg aggaaacctg cgtggagtgg ctgcggagat atgtggaata
cgggaaggct gagctgggca ggagagagcg acccgaggtg cgagtgtggg ggaaggaggc
cgacgggatc ctgaccttgt cctgccgcgc tcacggcttc tacccgcggc ccatcgttgt
cagctggctg aaggacggcg cggtgcgggg ccaggacgcc cagtcggggg gcatcgtgcc
caacggcgac ggcacgtacc acacctgggt caccatcgat gcgcagccgg gggacgggga
caagtaccag tgccgcgtgg agcacgccag cctgccccag cccggcctct actcgtggga
gccgccacag cccaacctgg tgcccatcgt ggcgggggtg gctgtcgcca ttgtggccat
cgccatcgtg gttggtgttg gattcatcat ctacagacgc catgcaggga agaaggggaa
gggctacaac atcgcgcccg acagggaagg tggatccagc agctcgagca cagggagcaa
cccctccatc tgagtgctgt gcttcagcct gcaaggagcc aacagtccac accagcattt
ggggtcggtg atggacacag ccccatcctc ttgacctctc agatgtccct ctgcttccta
tgctgactgt tattctttgc ctgcactgct tcctgtgaaa taaaatgatg ggccattctg
tg
SEQ ID NO. 3: major class I coding region in amino acids (B21 haplotype, B-
F2*2101, from AY234769, read signal sequence in italics, with start of mature
protein at beginning of second line)
MGSCGALGLGLLLAAVCGAAA
ELHTLRYIRTAMTDPGPGLPWFVDVGYVDGELFMHYNSTARRAVPRTEWIAANTDQQY
WDRETQIVQGSEQINRENLDILRRRYNQTGGSHTVQWMSGCDILEDGTIRGYHQAAYD
GRDFVAFDKGTMTLTAAVPEAVPTKRKWEEGGYAEGLKQYLEETCVEWLRRYVEYGKA
ELGRRERPEVRVWGKEADGILTLSCRAHGFYPRPIVVSWLKDGAVRGQDAQSGGIVPN
GDGTYHTWVTIDAQPGDGDKYQCRVEHASLPQPGLYSWEPPQPNLVPIVAGVAVAIVA
IAIVVGVGFIIYRRHAGKKGKGYNIAPDREGGSSSSSTGSNPSI
SEQ ID NO. 4: Class I minor gene (B21 haplotype, B-F1*2101 from ATG to TGA
including introns)
ATGCGCCCGT GCGGGGCGGT GGGCCTGGGG CTGCTGCGCC TGGGGCTGCT
GCTCGCCGCC GTGTGCGGGG CGGCGGCCGG TGAGTGCGGC CGGACCGGGA
CCCCTCCCCG CCCGTAACCC CACCCCGGGG CTGTGCCCGT GGGATCCTCA
GACCCCCACC CGCGGCTCAC GGCCCCGCTG CGCTCCGCCC CCGCAGAGCT
CCATACCCTG CGGTACATCC ATACGGCGAT GACGGATCCC GGCCCCGGGC
AGCCGTGGTA CGTGGACGTG GGGTATGTGG ACGGGGAACT CTTCGTGCAC
TACAACAGCA CCGCGCGGAG GTACGTGCCC CGCACCGAGT GGATGGCGGC
CAAGGCGGAC CAGCAGTACT GGGATGGACA GACGCAGATC GGACAGCGCA
ATGAGCGGAG TGTGAAAGTG AGCCTGGACA CACTGCAGGA ACGATACAAC
CAGACCGGCG GTGAGCACGG CCGGGGCCGC GGCTCCGTGG GTGTGGGATG
GGCTCCATGG CGCAGTGCCG CCCACACCCC CCAGGCCTGG CCCTGCCCGG
CGGCACCGTC CCGGGGCTGC CCGTCACAGC CCCACCGCGC TCGGGGTGCC
GCGTCCCGGG GGGACCCCAA CCCATCCCCG CTGCAGTGGG AGCCCCGGAG
CCGGAGGGGC CCCTCACCCC CTGCCCGGCT GTGTTTCAGG GTCTCACACG
GTGCAGTGGA TGTTCGGCTG TGACATCCTC GAGGATGGCA CCATCCGGGG
GTATCGTCAG GTGGCCTACG ATGGGAAAGA CTTCATTGCC TTCGACAAAG
ACATGAAGAC GTTCACTGCG GCAGTTCCAG AGGCAGTTCC CACCAAGAGG
AAATGGGAGG AAGGAGGTGT TGCTGAGGGG TGGAAGAGTT ACCTGGAGGA
AACCTGCGTG GAGTGGCTGC GGAGATACGT GGAATACGGG AAGGCTGAGC
TGGGCAGGAG AGGTGAGTGG GGTGGGGGGG GGGCCGCGGT GTGGGGCTGG
ACGTGGGGCG GGGGCTCAGC GTGGGGAGCT CAGCCCGGCC CTCATTGCCA
CCTGCCTGCA GAGCGGCCCG AGGTGCGAGT GTGGGGGAAG GAGGCCGACG
GGATCCTGAC CTTGTCCTGC CGCGCTCACG GCTTCTACCC GCGGCCCATC
GTTGTCAGCT GGCTGAAGGA CGGCGCAGTG CGGGGCCAGG ACGCCCAGTC
GGGGGGCATC ATGCCCAATG GCGATGGCAC CTACCACACC TGGGTCACCA
TCGATGCGCA GCCGGGGGAC GGGGATAAGT ACCAGTGCCG CGTGGAGCAC
GCCAGCCTGC CCCAGCCCGG CCTCTACTCG TGGGGTGAGT GAGGGGATGT
GGGGCTGGGG GGCTGCGGGC TGCCCCTTCC CCTGCTGATG GCCCCGCTCT
CCCCCAGAGC CGCCACAGCC CAACCTGGTG CCCATCGTGG CGGGGGTGGC
CGTCGCCATT GTGGCCATCG CCATCGTGGT TGGTGTTGGA TTCATCATCT
ACAGACGCCA CGCAGGTAAA AGCAGAGGGG TGCAGGCGGG CAGTGGTGGC
AGTGGGGGGA TCTGGGTCCC CCTTGGGAGC CCTCAGCCTG GCTGTGATGT
GAACCTGTGT TGATTCATCT CTCTGTCTGC AGGGAAGAAG GGGAAGGGCT
ACAACATCGC GCCCGGTGAG TGATGAGGGC AGCGCTGTCC CCCACCTCTG
CCCAGTGCCA GGGCGGTCCT GGGGTCTGCA CTTTCTCCCA GGGTACCCAT
TCCTGGTGCT TGGGGCTGCT CCACGCCCCA TAGGGAGCAC AGGGCTGGGT
CTCACAGCTG TTCCTCCCTT ATAGACAGGG AAGGTGGATC CAGCAGCTCG
AGCACAGGTG CGGTGTGGGG CTGTGGGTTG GGAGGGGTCC GTGTGCTCTC
TGTGGTACTG CCCAGGGCTG GGCTATGCTG GGGCTCTGCG GGGAGACCCC
CGGAGCAGAG GGTTGGGATG TGAACATGGG CCCCGTGGGA CACCATCTCT
TCTCATCCCC ACAGGGAGCA ACCCCTCCAT CTGA
SEQ ID NO. 5: B-NK coding region (cDNA) in nucleotides
(B21 haplotype from ATG to TAA)
ATGGATGAGG AAATTACTTA TGCTGATTTA AGGCATCCTA CGGGCAGTTT
GCCTCCTGCT AAGCGGCAGC GCGTTCCGTG GGACTTCCAG AAAGCCTGCA
TGCTGGGGGT GCTGCTGCTG CTGCTGCTGC TGCTGGTGAC AGGGCTCAGC
ATGTGCGTCT TTCAGAAAAC AACACCACCT CCAAGCGCTG CCCGGCACTG
CCCTGAGACC AACGGGAGGA ATGGGACAGA GCTCTGCAAG AATACCTCCA
TTGAGCAGTA CTTCTGCCAA TCCAAATTGA GCAGCTCTGC AGCCCCTGCT
GCCTGCCTGC TCTGTCCCCA GTTCTGGAGG CTTTTAGGGG ACCGATGCTA
TGGGCTTTCC ACAGAAAAGG AGAACTGGAC ACAAGCAAAA ATGAAGTGTG
AAAATCTGCA GTCTCAGCTG GCTGTGCTCC GGAAGAAGGC AGAAAAGGAT
CACCTCCAGA AGATGGCAGG TGCGGAGCCG GTGTGGATTG GATTAGAAGT
GTCCACAAAT CAATTGAAAT GGGTGGACAA CTCCTCCTAC AACAGCACAG
AGTTTGATAA CCTTCCCGTG ATGGAGAATG GCTGCGGGAC CTTCAAGAAC
ACAAAAGTGG AGGGCGACGT CTGTAGTGGT GAACACAAGT GGGTCTGCCA
GAAAGAACCT TTACGTCTCC ATCCATAA
SEQ ID NO. 6: B-NK coding region (CDNA) in amino acids
(B21 haplotype, from Met to stop)
MDEEITYADL RHPTGSLPPA KRQRVPWDFQ KACMLGVLLL LLLLLVTGLS
MCVFQKTTPP PSAARHCPET NGRNGTELCK NTSIEQYFCQ SKLSSSAAPA
ACLLCPQFWR LLGDRCYGLS TEKENWTQAK MKCENLQSQL AVLRKKAEKD
HLQKMAGAEP VWIGLEVSTN QLKWVDNSSY NSTEFDNLPV MENGCGTFKN
TKVEGDVCSG EHKWVCQKEP LRLHP*
SEQ ID NO.7: B-lec coding region (cDNA) in nucleotides
(B21 haplotype, from ATG to TGA)
ATGGAGGAAG TGAGGGAATA TCCCCATGTT TTGGGCACAG AATGGAGCAG
GAGGGAAGGT CCCAGCCGTG GGGCGTGCGT TACCTTCCAG CTCACGATGG
CAGCAGTGTT CACAGTGCTG CTCATCACTG CTGTTGCCTT TGCAGTGCAG
GCATTCCAGC CCCATCCCCA GCCCTGTGCT CAGTGTCCCT TCGACTGGAT
TGGATTCAGA GGAAAATGCT ACTACTTTTC AGAGGATGAG AGCAATTGGA
CGAGCAGCCA GAACAACTGC TCTGCACTTG GTGCTTCCTT GGCTGTGTTT
GACAGCGCTG AGGACTTGAG CTTCACAATG AGACACAAAG GCAGCTCCCC
CCACTGGGTT GGCCTCTCCC GGGAAGGCAA AGAGCATCCA TGGGAATGGG
TGAACCGCTC TCCTTTGTCT CACCTGTTCC AGGTGCAAGG TGATGGTCTC
TGTGCATACC TGGGGGATGC CGGGCTCAGC TCCTCCCACT GCAGCGCGCG
GAGGAATTGG GTTTGCACCA AACCCGCGTT GCAAAAACCG AGGAAGAACT
TCTGCATCAG CACCTGA
SEQ ID NO. 8: B-lec coding region (cDNA) in amino acids
(B21 haplotype, from Met to stop)
MEEVREYPHV LGTEWSRREG PSRGACVTFQ LTMAAVFTVL LITAVAFAVQ
AFQPHPQPCA QCPFDWIGFR GKCYYFSEDE SNWTSSQNNC SALGASLAVF
DSAEDLSFTM RHKGSSPHWV GLSREGKEHP WEWVNRSPLS HLFQVQGDGL
CAYLGDAGLS SSHCSARRNW VCTKPALQKP RKNFCIST*
SEQ ID NO. 9: B-NK and B-lec promoter and 5′UT regions (B12 haplotype, taken
from our AL023516, reads 5′ from immediately after the ATG of B-NK to
immediately before the ATG of B-lec, likely not a bidirectional promoter)
TTTGGGGCTT CCACTTCACC TGCAAACCAA AACTTCCACC CATCAGCAGT
GCCCCAAAAG CAGAGACCCA TAAGGATATA TTTCTGGGTG CTAAAACTCA
CTGCAGTGAC CCAAATGAAA AGCAATTCCT CCCTCTGTGG CAATGATCGC
CCAACAGAAA ACAGCAAGGA GAAGTCCTAG CATTGCCAGG GATAGACAAT
GAGAGAGGAC TGAAATTAAA TGCAATGTAA TATAGCGGAA AAAAAACCCC
ACAACCCTCC CCCATAAAAA TAAGCAACAA CAGCCACATC AGTCCCAATT
TATGGAAAAA ACCAGTGCTT TCCCCCATGC AATCAGAACA ACTGCCACTG
CCTTTCATTG TTACCACCTG CAGTTAGATG CCCCCCTCCC CTTGGAATGA
AATTATCCCC CATGACTCAA AGCCTGGCTC AGCAAAAAGA TATACCCCCA
GACCCCCACC CCAACCCATT GCCTGCGTCC GTCTGATGCT GAATGCGAAA
ATAAATGTGA TGCATGGTCA CAAAATGGGC TTTTTCAGGG GGGATTTCCA
CGTGGATGCT GCAGGGCAGA TGGGGAAGGG GTGAGGGGAG ATGCCCACCA
GCAGAGTTCC CATGCAGGAC ACAGCAGTTT TGCTGCCAGC ACCAGGAAGC
AGCTTCCCCT CCTTTCCCTG CTGGGAAATC ACTCCTTTGG AATGTTTTTT
TTTTTCTGCT GCTCACCCAC ATTTTGCACA GGGCTGATCT TCCAGGTCAG
CCCAAACTCT GCATCCCCGC ACGGATAACC TCTCCCTCCC TAAGAATCAG
TGCATCCTGC CTGCCTGCAA AGCAGCTGCT GAGATGTCTT TTGCAGCCCT
TATTCCCGCA GACCCCATGC AGAACCACAC ACATCTCCAT CCTCTCCTCC
GTTGGCGAGG AATGGGTTTG CAAAGGGATG GGCACAACCA GCAATATGCA
AAGGAAGAGG TGTCGAAGGT CTGGGGAGCA ATGAATCTGT CCCCCGGAAG
ATGTTTCCAT GGGGCAGTTA AGGAGGAGAA TTGGAAATGA AGCAGATGAT
GCAGCAATGA AACTATCCCA GAAAAGGGGG AAAAAGCAAT TCTGGTAATG
AAGATACATA AAGGAGAAGG GCTTCTCGCT GTCTGGACGC AGTTCTGTTG
GTTAACGTCT TTTCTCTTTG TGCTCTTTGC ACTTTTTTCT TTGCCTGCTC
TGGTCAGGAT GAGGCAGAGC CCTCACGGGG CCCTTTCACA CCTTTTTTTA
GCACACAGAA GCGCAGCGGC CGTCTCAGCA CCCAGCATGG AAGAGAAGGG
ACTGCAAATA AATTAAATGC GTTACTGAAT AGACAGCCGT AATTAAAAGT
CAAACCCATC CCCTCCCAGT ATTCCAGCTG CCGAGGCATC GGTTGGCACA
GAATCACCAA ATATTGCCTT TCTTCCCCCA TCCCCGCTTA TCAGCCAATG
CTCTCTGACC CCTAAAAGGT CTCGATTTGG GGTCTTTTTG TTGTTGTTGT
TGTTGTTCTG GGTATTTTTA GGCTTTTATT ATCAGCGATT TTTCAGCTTC
TCACTGCTTA CCCCCCAGCT CAGCACCGCG TCGCTCACTG CCATCGCTGA
ACCCAGCGGC GTTTCCATCC CTCAGAGAGC AGCAAAATGA GACATCGGCC
GTCGTGCACG GAAGGGAAAG GCTGTAGGGT ACATCTACCC TTATTTCTTG
GGTTTGTGTT TTGTTTTGTT GTTATTTAAA AGCAAAACCA AGACAACAAA
GCCCAGCCAA TGCCATTTCC TGGCAGTGGA CGCAGGCGCA GGCGGGTTGG
TCACAAAGCA AGAAGTTGCT GCGGGACTTT GTCGTTTTGG GGCCGTTCTC
GTGAACTTCT GAGCC
SEQ ID NO. 10: B-NK 3′UT (B12 haplotype, taken from our AL023516, reads 5′
TAA of B-NK gene to polyA signal)
tgtgcaggagatggagggcaacgcagtccctccctctgatgtgtgcccaccccaaaagccccaggcgttggggtgaa
gctctgcaatacggctctgtgaggagtttgggctgtgcagaccccagcactgcccgggcacacggggggacatcgct
gcatgcgctcatgcaatcagctcacaatcctgtgttatttctctcAATAAA
SEQ ID NO. 11: B-lec 3′UT (B12 haplotype, taken from our AL023516 entry,
reads 5′ TGA of B-lec gene to polyA signal)
gcggctcccggacccgaacacgcgatgcaagaggaggaacccaaagcaaaagagctccgctttcagctgtgctcagt
agcaacaggagggcggtgcgctcctccagcccaggtccgacagtgccgcctatggggctgcgcggaccgaagcaaat
cccaggcggagcttcggctccaaattacatttttttgcaccgtctgactcctaatgaccgctaaaatcccaattttg
ggggctatccgtgcgctgcttgcaacgaccttcacccctgcgcgatgcagcagcaggtttggggggcggacggtggg
aaaatatccatttttcaccggtttttctccaaagggaaatactgggaaagcaatcagcccaaaggaccctgaaatcg
atgAATAAA
SEQ ID NO. 12: DMA coding region (cDNA) in nucleotides (B21 haplotype, from
ATG to TGA)
ATGGGGGCTG CGAGCGGGCG CGGGGCGCTC GTGGCAGCGC TGCTGGGGGC
GGCCCTGGGG AGCGTCAGAG CCGAGCCATC ACTGCACACT CTGTCCGAGG
TGCTCTTCTG CCAGCCGGAC ACGCCGTCGC TGGGGCTGTC AGTGGCCTTC
GACTCAGAGC AGCTCTTCTC ATTCGATGTC CCCAACTCGC AGTGGCTGCC
GCAGCTCCCC GATGGCCCCT CGTGGCCCGC AGACATCGAG CAGCCCCACG
AGCTGCTGCA CGACGCCGCG CTGTGCCGTG AGCTGCTCGA TTTGCTCACC
AGAATCGCCA CCGGGCCAAA CCCAATGCCT GAAGCCAAGG GCATCCCGGT
GGCCGATGTC TTCCTGCAGC AGCCTCTGCA GCTCGGCTAC CCCAACACTC
TGATCTGTAT GGTGGGCAAC ATCTTCCCCC CAGCCATCAC TATCAGCTGG
CAGCGGGATG GCATCCCCGT CACCGATGGC GTCACCCACC TCACCTACAC
CCCCACCGAG GACCTGGGCT TCATGCGCTT CTCCTACCTG GCGGTGACAC
CGCACTCTGG TGACATCTAT GCCTGCATTG TCACCCGCGA GAGGGACAAC
ATCTCTGTGG TGGCTTACTG GGTGCCACAG GACCCCATCC CTTCGGACGT
GTTGGCCACG GCGGTGTGCG GCGCAGTGAC GGCGCTGGGC ATCCTGCTGG
CACTGCTGGG TTTGGGGCTG CTGCTGTCCG CCCGCCGGCG CAGTATGTGG
GGACAATGGA GACAGCAGGG ACACCCGCCC CGTACTCACT GA
SEQ ID NO. 13; DMA coding region (cDNA) in amino acids (B21 haplotype reads
from Met to stop)
MGAASGRGAL VAALLGAALG SVRAEPSLHT LSEVLFCQPD TPSLGLSVAF
DSEQLFSFDV PNSQWLPQLP DGPSWPADIE QPHELLHDAA LCRELLDLLT
RIATGPNPMP EAKGIPVADV FLQQPLQLGY PNTLICMVGN IFPPAITISW
QRDGIPVTDG VTHLTYTPTE DLGFMRFSYL AVTPHSGDIY ACIVTRERDN
ISVVAYWVPQ DPIPSDVLAT AVCGAVTALG ILLALLGLGL LLSARRRSMW
GQWRQQGHPP RTH*
SEQ ID NO. 14: DMB1 coding region (cDNA) in nucleotides (B21 haplotype, reads
from ATG to TGA)
ATGGGCTTTC TGGGGTGCAA CATGTGGGTT TGGGGGTGCA GCATGGGGGT
TTTAGGGATG CAGTGCGCCT TCGTGGTGCA CATGGCCAGC TCCTGCCCAC
TGCTGGCCAA TGGCTCCCTG GGCGGCTTCG ACCTCACCGT GGCCTTCAAC
AAGAACCCTC TGCTGTGCTA CGACCCCGAC GTCCACCGCT TCTACCCCTG
CGATTGGGGG CTGCTGCACA CCATTGCCAC TTTGCTCGCC GCCATCCTAA
ATGATGATAC CACATGGGTG CAGCGTGCAG AGGCACGCAG GCAGGCGTGC
ACTGAGCTGG CTGCACAGTT CTGGACACAC ACAGCACTGC GCAGGACACC
ACCCCAGGTC CGCATCGTCC CCATCCCCAT CTCCAACGAC CCCGACACCG
TCCACCTCAT CTGCCATGTT TGGGGCTTCT ACCCACCCGC AGTGACCATC
CAGTGGCTGC ACAACGGCCT CGTGGTGGCC TCAGGTGACA CCAAACTGCT
GCCCAACGGG GACTGGACCT ACAGGACACA GGTGGCCCTG AGGGCCAGCA
CTGCAGCAGG GAGCACCTAC ACATGCTCAG TGTGGCACTC CAGCCTGGAG
CAGCCGCTGC AGGAGGACTG GAGTCCCAAT TTGTCCCCGG CGATGATGGT
GAAGGTGGCA GTGGCGGCCA TGGCGCTGAC GTTGGGGTTG GTGGCACTCA
GCGCCGGGGT TTTCAGCTTC TGTCAGCGGC CACGGGTGTG GGGTGTACCT
CCTCATTCAT CATCTTCTTC CCCATCCTGA
SEQ ID NO. 15: DMB1 coding region (cDNA) in amino acids (B21 haplotype, reads
from Met to stop)
MGFLGCNMWV WGCSMGVLGM QCAFVVHMAS SCPLLANGSL GGFDLTVAFN
KNPLLCYDPD VHRFYPCDWG LLHTIATLLA AILNDDTTWV QRAEARRQAC
TELAAQFWTH TALRRTPPQV RIVPIPISND PDTVHLICHV WGFYPPAVTI
QWLHNGLVVA SGDTKLLPNG DWTYRTQVAL RASTAAGSTY TCSVWHSSLE
QPLQEDWSPN LSPANMVKVA VAAMALTLGL VALSAGVFSF CQRPRVWGVP
PHSSSSSPS*
SEQ ID NO. 16: DMB2 coding region (cDNA) in nucleotides (B21 haplotype, reads
from ATG to TGA)
ATGAAACTCT GGGGGGGGGG GACGTGGGGG TCTCTGCTTT ATGGGACTGT
GCCTTCATGG TGCATGTGGC CAACTCCTGC CCACTGGCAG CCAATGGCTC
TCTGCGCGGC TTCGACCTCA CCGTGGCCTT CAACAAGAAC CCTCTGGTGT
GCTACGACCC CGATGGCCAC CTCTTCAACG CCTGCGACTG GGGGCTGCTG
CACGGCGTGG CTGGACAGAT TGCCATTGCC CTCAACAATG ACAGCACCTG
GGTGCAGCGT GCAGAGGCAC GGAGACGGGC GTGCAGCAAA CTGGCTGCAC
AGTTCTGGGC ACAGACGGCG CTGCGCAGGA CCCCAGGTCC GCATCGTCCC
CGCACAGACA GGGAACCCCA GCGTGCCCAT CCGCCTCACC TGCCACGTGT
GGGGCTTCTA CCCCCCCGAG GTGACCATCA TCTGGCTACA CAATGGGGAC
ATCGTGGGGC CTGGAGACCA CTCGCCCATG TTCGCCATCC CCAATGGGAA
CTGGACCTAC CAGACACAGG TGGCCCTCTC GGTGGCCCCA GAGGTGGGGG
ACACCTACAC GTGCTCGGTG CAGCATGCTA GCTTGGAGGA GCCCCTCCTG
GAGGACTGGC GTCCTGGGCT GACGCTGGAG GTGACGCTGA TGGTGGCTGT
GGCCACTGTA GTGATGGTGT TGGGGCTCAG CTTGCTCTTC ATTGGTGTCT
ACTGCTGGCG GGCCCAACCC CCTGCCCCAG GTTACGCCCC GCTTCCCGGT
CACAACTACC CTTCAGGCAG CATCTGA
SEQ ID NO. 17: DMB2 coding region (cDNA) in amino acids (B21 haplotype, reads
from Met to stop)
MKLWGGGTWG SLLYGTVPSW CMWPTPAHWQ PMALCAASTS PWPSTRTLWC
ATTPMATSST PATGGCCTAW LDRLPLPSTM TAPGCSVQRH GDGRAANWLH
SSGHRRRCAG PQVRIVPAQT GNPSVPIRLT CHVWGFYPPE VTIIWLHNGD
IVGPGDHSPM FAIPNGNWTY QTQVALSVAP EVGDTYTCSV QHASLEEPLL
EDWRPGLTLE VTLMVAVATV VMVLGLSLLF IGVYCWRAQP PAPGYAPLPG
HNYPSGSI*
SEQ ID NO. 18: Promoter and 5′UT of DMA (B12 haplotype, taken from our
AL023516, reads from about 1 kB upstream of DMA ATG)
CAGGGGGCAT GTGGTGGGGG GAGGGAGGGG TGGGGCTGTG GGGATGCCCG
GTGTTGCTGG GGGGCTGCTG TAGGGTTGCA TGGCATTGCG AGGATGCAGC
CATGAAGATT CACGGCATTG TAAGTGTGCA TCTGTAGGGG CCCCTGGCAT
TGCAAGTGTG CACCTATGGG AGTGCCCGGC ATTGCAAGGG TGCACCTCTG
GACGCGTTTG TCATTGCAAA GGGTGCAGCT GTGGTGGTGA ATGGCATTGC
AAGGGTGTAT CTATGGGAGT GTACAGCATT GCAAGGGCGC ACCTATGGGT
GTGCCTGGCA TTGCATGCAT GCACGTGTGG GGATGTATGG CACTGGGGGG
GTGCACCAGT GGGGGTGCTT GGGATTGCAA GGGTGAGCCT ATAGCAGTGC
CTGGCATTGC AGGGTTGCAC GCAGGGATGC GTACGGCATT GCAGGGGTGC
AGCTGCCGGC ATTGCAGAGG GCCGAACCCG CCCGTACGGT TGTGCAGCGC
TTCCAGCTCG GAGGGCGCAT TGCAGTGCGG TGCATTGCAG TGCGAGGAGC
CACTGCTGCA GGGTGTACAG TGCACGCCCC GAGGATGTCC CCTCGGCTCC
AAACCCCAAA ACCCCACGCT TATTACCCCC CAAAAACATA CTTTTACACA
CAAGACACAT TTTTACCATC AAACCTCACG CTTTTCCCCC AAAATCCCTC
ACAAAAACAA AATCCGCGCC GTGATGAGAC ACCCCAGAGA TCTACGGAGC
CTACTCGTCC CCTGCTTCAT TAATTAGAGC TGCTTATTAA TTGCTTGCGG
GTGGCTCAGC GCCTATTACA GCGTCGGGGC TCCCCGCTAG TTTCTTCTAT
CTAGTAACAA GTGACGCAAG GTAACTGCGG AGCGCGGCCA TTGGTTGAGC
CGCACGATCA TCTCCTGTCA CAGCGCTGGT GTTCCCCGCA GATCTGTTCT
GCCTAGCAAC CGATGACGCG TAAAGCCGCG AGGCACGGCC ATTGGCTAAA
CTGGTTGCCG GTAGCAGAGG G
SEQ ID NO. 19: 3′UT of DMA, intragenic region including promoter of DMB1 and
5′UT of DMB1 (B12 haplotype, taken from our AL023516, reads from just after
TGA of DMA to just before ATG of DMB1)
TGTCCCCCAT AAGTTGATCC CTCGGTGTGG GAACGGTGAT GGTGATGTAA
TTAAAGCCCT TCATTTGCAG CGCGGTGTCC TTGTTTGTCC CCACTCCGGG
AAGGGTGGCA ATTAATGGGG TTGGGCATTG TCCCCATGGC CCCAGGTGGC
AAAGTCTGAT CCCATTGCAC CGCCCATGGG GTGACGTGAG GAGGAGGAGG
AGGAGGAGGA GAAGGAGTAG GAGGAGGTGA AGGTGGCACG GAGGGGATGA
AGGCAGCAGT GCTCCTGATG GGGCCAAGGG GTTTCAGGGT GCTGGGGGCG
ATGGGGCTGG TGCTGAGCTG CGGGACAGCA GGTAGGATGT GGGGAAGTGT
GGGGTTTTTG GGGTGAAGCA TGGGGGTTTT AGGGTGCAAC
SEQ ID NO. 20: 3′UT of DMB1, intragenic region including promoter of DMB2 and
5′UT of DMB2 (B12 haplotype, taken from our AL023516, reads from just after
TGA of DMB1 to just before ATG of DMB2)
TGCGGGTTCT CACTCCAATC CTGGTCCCCA AAATGATCCC GGTCCAAGTT
CTGGTCCCCA TCCCAGTCCT GGTCCCCATT CTGGTCTTGG TCCTGGTCCT
GGTTCTGCTC CTGGTCCCTA TCCCTGACTC TGGTCCCGGT CCCCATCCCG
ATGCCAGTCC CAGTCCTGGT CCCCATCCTG GTCCTGCTCC TTGGTTTGGG
GACCTCAATG ACTGGAACTC CCATGTCCCA ACATGGGGAC CCACAGTTTG
GGGTGAGGGG CTCTCACCCC CCAATAAAAC CATCTGCAGC CCCAACCTCG
CTCCAATTCT TCGTTCCCAC GTTGGGTGGG TCGGGCTCCC AGTGCTCCCA
GTGCTCCCAG CCGTCTATGT CCCGTAAGCG TCGGCTCCAC TGCATTCTGC
TCCGGAAACA GATGACGCTA CCACGGCGCC GCCTCTGATT GGCTGCTCCG
TGCCCTCTCT CCGTCCCACG TCCGTGAAGG GGGGGATGTG GGGTGGAGGG
AGCTGAGGGG CCGCCCCCTT CCCCCCCCCG CTCCCCCCTC CGCGATGTTG
GTGCTATTGG GGCTGCTGCT GGGAGCGCGG GGGGCAGGTG GGGGTTTGGG
GTTGGGGTGT TTGGGGGGTC TCTGCCTA
SEQ ID NO. 21: 3′UT of DMB2 (B12 haplotype, taken from our AL023516, reads
from just after TGA of DMB2)
TGGACACCTT CTGTCACCAA CTGTCCCTGC GTGTCCCCAT CCCTGACTCT
GCGCCGTGGT GCTGACATTA AAGACACTCT GCAGCCTCTG TTGGTGTCTC
TGTGGGCTTT TGGGGTGGGG TGGTGTCACC GGGGAGAGGT TGGGTTGGGG
TCATTGCATC CATGATGGTG ATGGTGATTG ACATTGTGCA CAGGGAGATG
TCCAGGCGCC TGTGGGGTCT GTGTTTTAGG GCCAGTTCTG CTCAGTGCCT
CCGTAAGTGA TCTGGATAGG TCGTCAGTCA TCCTAATTAA GGAGGGGACA
ACAGTGAATG GGGAGGAGCC GATGACTCAG GCTGGGAGTG GTGATCCCAG
AGGTTTCCTC TGCTGTCAGT GACTCCGTGC TTTCGCTTTC GCTTCACAAC
CTGAGGGAGC GCATTCTGCC TGGCGCCCGA TGACGTCACA TAAACCCCCG
ACTGCCATTG GCGGAGAGGC GACGGAGGAG CCAATGGGGG CGCGGGGCGG
GGCGGAGGAG TAGGAAAAGC TGAAGGAGCT GCGCTGGGTG CGGCGGACTT
GAGAGTGCAG CGGTGTGAGG CG
SEQ ID NO. 22: TAP1 and TAP2 promoter and 5′UT regions (B21 haplotype, reads
5′ from immediately after the ATG of TAP1 to immediately before the ATG
of TAP2)
CGCGGCCCCGATCCCCCTCCGGCCCGATCCCAATGCCCGGCAGCGGCCGG
AGCTTCTCGGAAACGAGAGCGTCTCTCATTGGCTGAGGCGATGCAGCAGC
GACGCTGCTCATTGGCCGAGATGGTTCCGCGTCATCAGTTGCCAGGCAGA
TCGGAACACTGCAGTTTGGAGAGGGGCGGGTAATTGAAAGTGAAAGTAAT
GGCGGAGCGGGGAGGAGATGGAGAGCGGCGGCGGTGAGGGGCTGGAGGGG
GCTGGAGAGGGCTGGACTGGTAGGGGCTGGTGGGGTCTGGTGGCCACTGG
TGGGGTCTGGTGGTTACTGGTGGGGACTGTCGAGGGGCTGGAGGGATCTG
ATGGGGACTGGAGGGGTTTGGTGATGGCTGTTGTGTGCTCCAGGCTGGGC
TGTGGGGAGCCGGACTGAAAGTGGGGGCCGTTCTAAAAGCACTGCTGTGT
GTTCCAGGTGCTGAGGGGAGCTGAGGACCTGCACCAGGAGCACCCCGGGG
AGCCCACCTGGTCCAGCTGTGCACCAGAAGCTCTGGGGATCCCCACCCCA
CAGCC
SEQ ID NO. 23: TAP1 coding region (cDNA) in nucleotides (B21 haplotype, reads
from ATG to TAG)
ATGGGGACGATGGGCGCGGGTCGCCGCCTCCTCCTCTCGCTGAGCCCGGA
GCGCCGGCGCTGCGCCGCGGTGATGGGTCTGATGGCGGCTTCAGCGCTGG
GCGAGATGGCCGTGCCCTACTACATGGGGCGAGCCAGCGACTGGGTGGCC
CGTGAGGACGAGCTGGCAGCCATCCTGCCCATGGTGCTGCTGGGCCTCAG
CAGTGCTGTCACTGAGCTGGTGTGCGATGTGACCTTCGTGGGGACACTGA
GCCGCACGCAGAGCCGCCTCCAGCGCCGCGTCTTCGCCGCCGTCCTGCGG
CAGAGCATCACCGAGCTGCGCGCCGACGGGGCCGGGGATGTGGCCATGCG
GGTGACGCGGGATGCGGAGGACGTGCGCGAGGCTCTGGGTGAGGCGCTGA
GCCTCCTGCTGTGGTATCTGGCGCGCGGCCTCTGCCTCTTTGCAACCATG
GCCTGGCTGTCCCCGCGCATGGCGCTGCTCACCGCGCTGGCGCTGCCGCT
GCTGCTGGCCCTGCCCAGGGCTGTGGGGCACTTCCGGCAGGCCCTGGCAC
CACAGATGCAGAAGGCGCAGGCCCGGGCCAGCGAGGTGGCAGTGGAGACC
TTCCAGGCCATGGCCACTGTGCGAAGCTTTGCCAATGAGGATGGGGCAGC
TGCACACTACCGGCAGCGCCTGCAGCAGAGCCACCGCCTGGAGAAGAAGG
ATGTGGCCCTCTACACTGCCTCTCTCTGGACCAGTGGTTTCTCAGCCCTG
GCCCTGAAGATGGGGATCCTCTACTATGGGGGGCAGCTGGTGGCCGCGGG
GACCGTCAGCACTGGGGACCTCGTCACCTTCCTCCTCTACCAGATACAGT
TCACTGATGTCCTGAGGGTCCTGCTCGACTACTTCCCCACACTGATGAAG
GCTGTGGGCTCTTCGGAGAAGATCTTTGAGTTCCTGGACCGGGAGCCACA
GGTCGCACCCTCAGGGACAATGGCACCCACTGACCTGCAGGGCCACCTCC
AGCTGGAGGATGTCTGGTTCTCCTACCCTGGGCGCCAGGAACCCGTCCTC
AAGGGCGTATCACTGGAGCTGCGCCCCGGGGAGGTGCTGGCACTGCTGGG
ACCCCCGGGCGCAGGGAAGAGCACTCTGGTGGCCCTCGTGTCCCGCCTGC
ACCAGCCCACGGCCGGACGCCTGCTGCTGGATGGCCACCCCCTCCCCGCC
TACCAGCACTCCTACCTGTGCCGCCAGGTGGCCGTCGTCCCCCAGGAGCC
GCTGCTCTTTGCCCGCTCACTCCACGCCAACATCTCCTATGGGTTGGGGG
GCTGCAGCTGGGCACAGGTGACAGCGGCCGCCCGCCGGGTGGGCGCCCAC
GACTTCATCACTCGCCTGCCCCAAGGCTACGACACAGAGGTGGGCGAGTT
GGGAGGACAGCTCTCCGGGGGGCAGCGGCAGGCGGTGGCCATTGCCCGTG
CGCTGCTGCGGGACCCCCGCATCCTCATACTCGACGAGCACACCAGCGCC
CTGGACACTGAGAGCCAGCAGCAGGTGGAGCAGGAGATCCTCGCAGCCAA
AGGGTCGGGGCGTGCAGTGCTGATGGTGACGGGGCGGGCAGCCCTGGCGG
CGCGGGCACAACGAGTGGTGGTGTTGGAGGGGGGAGAGGTGCGGCAGGAG
GGACCCCCCCACGAGGTGCTGCGCCCCGGCAGCCTTCTGCGGGACTGGGG
ACAACAGGGAGCACCGGGGGAGGGGGACAGAGGGAGTGGGGGGGAGGGAT
AG
SEQ ID NO. 24: TAP2 coding region (cDNA) in nucleotides (B21 haplotype, reads
from ATG to TAG)
ATGCCGCCCTACATTCTGCGCCTGTCATGCACGCTGCTCCTGGCCGACCT
GGCCCTCATGTTGGCCCTGGCCCACTTCTTCCCAGCACTGGCCCATTTGG
GCTGGGTGGGCTCCTGGCTGGAGGCCGGGCTGCGCCTCCTGGTGCTGGGG
GGGGCCGGGCAGCTGCTGGCCCCCAGGGGACCCCATGGGGCTGCGGTGCT
GCTGAGCCTGGGCCCCGCCATCTTCCTGACCCTACGGGGCTATGTAGGTC
TGCCTGGAGCTGCCCCGGTGCTGCTGGCCATGGCAACGCCGTCCTGGCTG
GTGCTGACCCACGGGACAGCTGTGGTGGCATTGCTCACCTGGAGCCTCCT
GGTCCCCACTGTGGCCACTGGGGCAAAGGAGGCAGAGGCCTGGGTGCCCC
TGAGGCGGCTGCTGGCCCTCGCCTGGCCCGAGTGGCCCTTCCTTGGCTGT
GCCTTCCTCTTCCTCGCATTGGCTGCACTGGGTGAGACCTCAGTGCCCTA
CTGCACCGGGAGGGCTCTGGATGTCCTCCGCCAGGGGGACGGCCTCGCCG
CCTTCACCGCTGCTGTCGGCCTCATGTGCCTGGCCTCTGCCAGCAGCTCG
CTGTTTGCCGGCTGCCGCGGTGGCCTCTTCACCTTCATCAGGTTCCGCTT
CGTCTTGCGCACCCGCGACCAGCTCTTCTCCAGCCTGGTGTACCGGGACC
TCGCCTTCTTCCAGAAGACCACAGCAGCTGAGTTGGCCTCCCGGCTGACC
ACCGATGTGACGCTGGCGAGCAACGTGTTGGCACTCAATATCAACGTCAT
GCTGAGGAACCTGGGGCAGGTGCTGGGGCTCTGCGCCTTCATGCTGGGGC
TGTCCCCGCGCCTGACAATGCTGGCACTGCTCGAAGTGCCGCTCGCCGTC
ACCGCACGGAAAGTCTATGACACCCGGCACCAGATGCTGCAGCGGGCCGT
GCTGGATGCAGCAGCCAACACTGGAGCGGCAGTGCAGGAGTCCATCTCTT
CCATTGAGATGGTACGGGTCTTCAATGGCGAGGAGGAGGAGGAGTACCGC
TACAGCCAGGTGCTGGACAGGACCCTACGGCTGCGGGACCAGCGGGACAC
AGAGAGGGCCATTTTTCTCCTCATCCAGCGGGTGCTGCAGTTGGCCGTGC
AGGCACTGGTGCTATACTGTGGGCACCAGCAGCTCCGCGAGGGGACCCTC
ACTGCCGGCAGCCTCGTCGCCTTCATCCTCTACCAGACTAAAGCTGGCAG
CTGCGTGCAGGCACTGGCGTACTCCTATGGTGACCTTCTGAGCAATGCAG
TGGCCGCCTGCAAGGTCTTTGATTACCTGGACTGGGAGCGAGCTGTGGGT
GCTGGTGGCACCTATGTGCCCACCAGACTGCGGGGCCACGTCACCTTCCA
TCGGGTGTCCTTCGCCTATCCCACTCGCCCTGAGCGCCTCGTCCTGCAAG
ATGTCACCTTCGAGCTGCGCCCCGGTGAGGTGACGGCGTTGGCGGGGCTG
AATGGCAGCGGGAAGAGCACCTGCGTGGCACTGCTGGAGAGATTCTATGA
ACCTGGGGCTGGGGAAGTGCTGCTGGACGGGGTGCCGCTGCGGGACTACG
AGCACCGCTACCTGCACCGCCAGGTGGCACTGGTGGGGCAGGAACCCGTG
CTCTTCTCTGGCTCCATTCGGGATAACATTGCCTACGGGATGGAGGACTG
CGAAGAGGAGGAGATCATAGCAGCTGCAAAGGCTGCAGGTGCTTTGGGCT
TCATCTCTGCACTGGAGCAAGGCTTTGGCACTGACGTAGGGGAGAGAGGG
GGGCAGCTGTCAGCGGGGCAGAAGCAGCGCATCGCCATTGCCCGCGCTTT
GGTGCGGCGTCCCACCGTCCTTATCCTCGACGAAGCCACCAGTGCTCTGG
ATGGGGACAGCGATGCAATGCTGCAGCAGTGGGTGAGGAATGGAGGGGAC
CGGACGGTGCTGCTCATCACCCACCAACCACGGATGCTGGAGAAGGCAGA
CCGCGTTGTGGTGCTGGAGCATGGCACGGTGGCTGAGATGGGGACACCCG
CCGAGCTGAGGACCCGCGGCGGACCCTACAGCCGGCTGCTACAGCACGTG
A
SEQ ID NO. 25: TAP1 coding region (cDNA) in amino acids (B21 haplotype, reads
from Met to stop)
MGTMGAGRRLLLSLSPERRRCAAVMGLMAASALGEMAVPYYMGRASDWVAREDELAAILPMVLLGLSSAVTELVCDV
TFVGTLSRTQSRLQRRVFAAVLRQSITELRADGAGDVANRVTRDAEDVREALGEALSLLLWYLARGLCLFATMAWLS
PRMALLTALALPLLLALPRAVGHFRQALAPQMQKAQARASEVAVETFQAMATVRSFANEDGAAAHYRQRLQQSHRLE
KKDVALYTASLWTSGFSALALKMGILYYGGQLVAAGTVSTGDLVTFLLYQIQFTDVLRVLLDYFPTLMKAVGSSEKI
FEFLDREPQVAPSGTMAPTDLQGHLQLEDVWFSYPGRQEPVLKGVSLELRPGEVLALLGPPGAGKSTLVALVSRLHQ
PTAGRLLLDGHPLPAYQHSYLCRQVAVVPQEPLLFARSLHANISYGLGGCSWAQVTAAARRVGAHDFITRLPQGYDT
EVGELGGQLSGGQRQAVAIARALLRDPRILILDEHTSALDTESQQQVEQEILAAKGSGRAVLMVTGRAALAARAQRV
VVLEGGEVRQEGPPHEVLRPGSLLRDWGQQGAPGEGDRGSGGEG
SEQ ID NO. 26: TAP2 coding region (cDNA) in amino acids (B21 haplotype, from
the Brian Walker thesis from our lab, reads from Met to stop)
MPPYILRLSCTLLLADLALMLALAHFFPALAHLGWVGSWLEAGLRLLVLGGAGQLLAPRGPHGAAVLLSLGPAIFLT
LRGYVGLPGAAPVLLAMATPSWLVLTHGTAVVALLTWSLLVPTVATGAKEAEAWVPLRRLLALAWPEWPFLGCAFLF
LALAALGETSVPYCTGRALDVLRQGDGLAAFTAAVGLMCLASASSSLFAGCRGGLFTFIRFRFVLRTRDQLFSSLVY
RDLAFFQKTTAAELASRLTTDVTLASNVLALNINVMLRNLGQVLGLCAFMLGLSPRLTMLALLEVPLAVTARKVYDT
RHQMLQRAVLDAAANTGAAVQESISSIEMVRVFNGEEEEEYRYSQVLDRTLRLRDQRDTEPAIFLLIQRVLQLAVQA
LVLYCGHQQLREGTLTAGSLVAFILYQTKAGSCVQALAYSYGDLLSNAVAACKVFDYLDWEPAVGAGGTYVPTRLRG
HVTFHRVSFAYPTRPERLVLQDVTFELRPGEVTALAGLNGSGKSTCVALLERFYEPGAGEVLLDGVPLRDYEHRYLH
RQVALVGQEPVLFSGSIRDNIAYGMEDCEEEEIIAAAKAAGALGFISALEQGFGTDVGERGGQLSAGQKQRIAIARA
LVRRPTVLILDEATSALDGDSDAMLQQWVRNGGDRTVLLITHQPRMLEKADRVVVLEHGTVAEMGTPAELRTRGGPY
SRLLQH
SEQ ID NO. 27: Tapasin gene (B21 haplotype, from intragenic region/promoter
to 3′UT, reads from -800 from ATG start codon of tapasin to 800 bp beyond the
TGA stop codon
B21LNtrim
B21LNtrim Length: 5000 16 Oct. 2003 13:40 Check: 2344 ..
      1 GGCCCTCGTC CCACCCGATG GTCCTCCAAT GACCTCCATG GTCATCCAGT
    51 GCTCATCCCG CGGTATGGCC ATCACCCTGC AGTCACCCAA TGGTCTTATC
  101 AAAGTTATGC CATGGTCATC TCTTTGTCAC CCCATTCTCA CTCCCTGTTA
  151 CTTCTGTTCC CTCTCGTTGT CCCCTAATGG TCACACCATT TTCTGCCCGT
  201 GGTCACTCAG CGTTTCTACC GTTACCCAAT CATGGTCACC CCGTGTTCCT
  251 CTCCGTGATC CCTTCATGGT CATCCCATAT CATCTACCTG GACTTGTGTG
  301 CGGCCATTGG GACGGTCCCA CGCCACATCC TTATCACTAA ATTGGAGATA
  351 AGTGTGCAAA GAGTGGACTT TGGATAAGGA ATCAGTTGGG TGGTCGCAGC
  401 CAGAGGGCTG TGGTCAATGG CTGCATGTCC CAGTGGGGAT GGCGACAAAT
  451 GGGGTCCCCG GGGGGTCCGT CTGGGAGCGG TGCTCCTCTG CATCTTTGTC
  501 AAAGACCAAT GAAGAGCACT GAAGACCACC AAAGACCACT GAAAATCACT
  551 GAAGATCACA AAGATCCCCC GAAGATCCCT CCATGCAAAC GCAGAAGGGT
  601 CCAATATGTG CTGGAACCTC ATCCTTACGT CACCCATGGA TCCGGGGTGG
  651 GTGGCAGTGG CTGTGTTTAG GTCGGCCTGT GGGGAAAGCC GGGCTGTCCC
  701 ACCCATGTCC CCTCTTCCAA CACTGTTCCT GAATGAGTTT TCCCTCTCCG
  751 ACCCTTTTTT TAATGGGTTT CAGGGATTTA AAATTAATAT TGACGAAGTG
  801 ACGGAGGGGG TGGGGCCACA GCGGAGCCGA AAGCGAAAGC AGCGGAGAGC
  851 GATGGCTGCG GGGCTGCGGC TGCTGCTGGC GGGTGAGACC CGACCCCCCC
  901 CGGCCCCCTC ATGTCCCACC ACCCATATCG CCCCCCCCCC CCGCCCTCGC
  951 CCCATGCTGA GCCTCTCCCC CACCCCCAGG GCTCTGCTGG TCCCAATTTA
1001 GGGTGGAAGA CGCCGCCTCC CCTCCGCCCC CCCCCGCTCC GGTGCGCTGC
1051 GCGCTGCTGG AGGGGGTGGG GCGCAGGGGA GGGCTGCCGG GGGGGGGCAA
1101 TGCCCGTCCT GCACTGCTGC GCTTTGGGGG GGACGCGGAG ACCCCTCCCG
1151 AACCCGGCCC GGAGCCCGAA GTCACCTTCA ATGTCAGCGG TACGTGGGGA
1201 CCCCCGGCAC TGTGCTGTGC GCCTCCTTTA TCCCCACCCC CCTCCATGTC
1251 CCCATCTCCT TTACTTCCCA CAATGCTCCC ATCGCCCCCA GAATGTCCCC
1301 AGAGTCCCCC AAACCCCCAT GACCCCCCCC CCACGATCCC CTGGTTCCCA
1351 TTACCCTCTC ACGTCTCCCA GTGTCCCCAA GATTCCCATT ACTCCCCGTA
1401 TCCCCATTAT CCCCAAAATG TCCCCCAATG TTCCCATCAC CCCAATGTTC
1451 CCAAGGTCCC TATCGCTCCT CAATGTCGCT ATGATCCCTA TTCCCAAAAT
1501 GTCACCAATG TCCCCAAAAT CCCCATTATC TCCCACCTCT CCAAAGTCCC
1551 CAAGATCCCC ATTACCCCCA ATATCCTCAT TACACCCCAA ATGTCCCCAA
1601 TGTCCCCTCC ATGTCCCCCA GAGACCCCAT TAGCCCCAAT AGCTCCCAAA
1651 CTGTCCCCAG TGTCCCCATT AACCCCAAAA TGACCCCATT ACGCCCCACA
1701 CCCCTCCCAA CCCCATGCCC TCAGACCCCT TCATCCCTCT CACTCCTCTC
1751 TCCCTCGCAG ACCCCTGGGG GACTCTGACC CCACTCGGGG TCCCCCCCCG
1801 GACTCCCCCC AGCTGCGAAC TGAACCCCAC GAACCCCCAG ACCGGCTCTG
1851 ACCCATGGAG CCGCCCTCTG CACCCCGACG CCCGCAGCCC CCCAACCGCG
1901 GGGGGGCAGT GGTGGGTGGC GGCGGTGGGG ACCCCGCAGT ACGGTGTCAC
1951 TGCGCTGCTG CAGGGGGGGA TGGGCACAGA AGGAACCATC ACTGCCGCCG
2001 GTAAGGGGGA ACTTGGGGTG TCCCTCCCTG GGTGTCCCCA TGTCCCTATC
2051 TGTCCCCCCG TGTGTCCCCA TTTGTCCCCT CCTCTGCATG TGTCCCAATG
2101 TCTCCATACA TCCCTATAAT AACCATATGT CCCCACTCAT CCCCATATTC
2151 CCCATGTGTC CCCATATCCC CACACATCCC AGTGTGCCCC AACACATCCC
2201 CATGTGCCCC CCCCCATGCA TCACTACCAT CCCCCTATCC CCCAAGTGTC
2251 CCTGTGTCCC TGCAGTTTCT CCCTGTCCTC ATGTGTTCCC ATGTCTCCAT
2301 GTCACTGTGT CCCCGTGTCC CCACACATCA CCATGCCCCC CATGCATCCC
2351 TGCGTCCCCC ACACATCCCC ATAATCCCCA TATTTCCTTA TCTCTCCATG
2401 TCTCTGCAGC GCCCCCATGT CCCTTCACCT CTCCATGTCC CCCAGTGTCC
2451 CCATATCCCC TCATTGTCCC CATGCCCCCT CACCTCCCCG TGTCCCCCGT
2501 GTCCCTATGT CCCCCTGGTG TTTCCATGTC CCCTCATGCC CCCATGTCCC
2551 CTCATGCCCC CATATCCCCC AGTGTCCCCA TGTCCCTTCA CCTCCCCATG
2601 TCCCCCAATA TTCCCATATC CCCTCACCTC CCCATTTCCC CCCGATGTTC
2651 CCATGTCCCC TCACCTCCCC ATGTCCTCAC AGTGGCCCTG GCGGTGCTCA
2701 CCCACACCCC GGCCCTCCGG GCCCGTGTGG GGTCCCCCAT CCACCTGCAC
2751 TGCGCCTTCG CCGCCCCCCC GTCCCCCTTT GTCCTCGAGT GGCGTCACCA
2801 GAACAGGGGT GCGGGGAGGG TCCTGCTGGC CTATGACAGT TCCACCGCCC
2851 GCGCCCCCCG CGCCACCCCC GGGGCCGAAC TGCTGCTGGG GACACGGGAT
2901 GGGGACGGGG TGACAGCAGT GACACTGCGG CTGGCACAGC CATCACCGGG
2951 GGATGAGGGC ACCTACATCT GCTCCGTGTT CCTGCCCCAC GGGCACACAC
3001 AGACAGTGCT GCAGCTCCAC GTCTTTGGTG CGTCCATGTG GGGCAGGCGG
3051 TGTTCCTATG GGGTGTGGGG TTGGGCAGTG TTCCTATGGA GTGTGTATGA
3101 CTGGGTGGTA TTCCTATTGG TCAGATAGGA CATATGGGAG CAGGCGGTAT
3151 TCCTATGGGG GCTGTAGGGT GGATGGGACT GGGTGATATT CCTGTGGGGG
3201 GCTGTAGGGT GGATGGGACT GGGTGGTATT CCTATGGAGG CTATAGGGTG
3251 GATGGGACCG GGTGGTATTC CTATGAGGAC TATAGGATGA GGTGGCATCA
3301 TCCCATAGTT CACCTGTAGG TTTATAGGGG GGGATGAGCC CTATAGGAGG
3351 AGCCCTATGG GCTATATGGG ACCGATGTCC CCCCACNNGT CTCCCAGAGC
3401 CCCCCAAGGT GACGCTGTCC CCGAAGAACC TGGTGGTGGC TCCGGGGATG
3451 TCAGCAGAGC TACGCTGCCA CGTGTCTGGC TTCTACCCCT TGGATGTGAC
3501 GGTGACGTGG CAGCGCCGCG CCGGGAGCTC GGGGACATCA CAGTCACCCA
3551 GGGACACAGT GATGGACAGC TGGACTTCAG GTCACCGCCA GGCAGCCGAT
3601 GGAACCTACA GCCGGACGGC GGCAGCACGG CTGATCCCCG CACGCCCCCA
3651 ACACCACGGG GACGTCTACA GCTGCGTTGT CACCCACACT GCACTGGCCA
3701 AACCAATGCG CGTCTCCGTC CGACTACTCC TGGCTGGTGA GGGGGGATGT
3751 GGGGATATTG GAAACACGTG GAGGTATTGG GATGCTGGGA CCGTGGTTAG
3801 GAGGGTCTGA GGGACATCAG GACCATGGCC TGGGACAATG GGAGATCATG
3851 GATTTGGGTT GGGGACCCCA CCCAGGATGG TGACACTGTG CTTAGGGCTG
3901 TCGTTGTCCC CACAGGCACC GAGGGACCGC ACCTGGAGGA CATCACGGGG
3951 CTCTTCTTGG TGGCCTTTGT CCTCTGTGGC CTCATCCGTT GGCTCTACCC
4001 TAAAGGTGAG TGCTGTTCCC ACATCCCAGT TCCCCCACAT CCTCACACCC
4051 CAATATCCCA ATGGCCCATG TCCCCATGAG CAATGTCACT ATGTCCCAAT
4101 ATCCTAATGA TGCTGTGTAC CCATGTGTCC CCATGTCTCT ATTCCACTCA
4151 CTCTTTCTCT CCCCTCAGCT GCACGACCCA AAGAGGAAAC CAAGGTAACA
4201 TCCCCCCCCA AAAACCCCAA ACCCCCCCAA AACACCTCCA AGCACCCCAA
4251 AACTCACCAT TCTCATTNCC CCCCCNCCCC CCATGCCTTG CAGAAATCGC
4301 AGTGACCTCC ACTCCAGCTC TCAGCACCTC CCTCCACTCC AGCTCTCAGC
4351 ACCTCAGCTC CAGATAAAGA GTTTTTCACC CCAAAGTTCT CTCTGTGTGG
4401 TGGTGTCCCC ACAGGTCTGG GTGCAGAGGG GGGAGAACTG GGGGCAAACT
4451 GGGAGCAGTG GGAGCAGTGG GAGGAAGTCC TGGGTTGGTG AGGCAGCTGA
4501 GTGACACCTG GGGACATCTG GGTGCCATCC CTTGTGGACA TCTGGGTGAC
4551 ACTGCGATTG CATTGGGTGA CATTGGGATC CCATCCCAAG CTGGACATCG
4601 GGATGCCATG AAGGATGGCC ACGTCCCATC ACATGTGGGC ATCTGGGTGA
4651 CATCGGGGAT GGTCATTTCC CAACCCATGG AGGCATCTGG ATGACAGTGG
4701 GGACACCTGG GTCCCATCCA ATGTGCACAT CTGTGTCCCA TGCCATATGG
4751 ACACCTGGAT GACATTGGGA ATGGCCAGGT CCCGCCCCAC TGAAGACATC
4801 TGGGTCCCAC TGAGGAAATC TGGGCCCCTT CAGGGACACC GGGGACATGA
4851 TTGGGTAATG GTGAGGTCAC TATAAAGGGA ACCCTGGAGG AATTTGTGCT
4901 GAACATGACC TGATCGCAGA GGGAAACGTC TGTTAACCAT GAGGGGATCA
4951 TGAAGGGGCA GAGGGACAGA AAGGGGGGAT ATGGGATGAG AATAGGGTAA
SEQ ID NO. 28: Tapasin coding region (cDNA) in nucleotides (B21 haplotype,
from ATG to TGA)
ATGGCTGCGGGGCTGCGGCTGCTGCTGGCGGGGCTCTGCTGGTCCCAATT
TAGGGTGGAAGACGCCGCCTCCCCTCCGCCCCCCCCCGCTCCGGTGCGCT
GCGCGCTGCTGGAGGGGGTGGGGCGCAGGGGAGGGCTGCCGGGGGGGGGC
AATGCCCGTCCTGCACTGCTGCGCTTTGGGGGGGACGCGGAGACCCCTCC
CGAACCCGGCCCGGAGCCCGAAGTCACCTTCAATGTCAGCGACCCCTGGG
GGACTCTGACCCCACTCGGGGTCCCCCCCCGGACTCCCCCCAGCTGCGAA
CTGAACCCCACGAACCCCCAGACCGGCTCTGACCCATGGAGCCGCCCTCT
GCACCCCGACGCCCGCAGCCCCCCAACCGCGGGGGGGCAGTGGTGGGTGG
CGGCGGTGGGGACCCCGCAGTACGGTGTCACTGCGCTGCTGCAGGGGGGG
ATGGGCACAGAAGGAACCATCACTGCCGCCGTGGCCCTGGCGGTGCTCAC
CCACACCCCGGCCCTCCGGGCCCGTGTGGGGTCCCCCATCCACCTGCACT
GCGCCTTCGCCGCCCCCCCGTCCCCCTTTGTCCTCGAGTGGCGTCACCAG
AACAGGGGTGCGGGGAGGGTCCTGCTGGCCTATGACAGTTCCACCGCCCG
CGCCCCCCGCGCCACCCCCGGGGCCGAACTGCTGCTGGGGACACGGGATG
GGGACGGGGTGACAGCAGTGACACTGCGGCTGGCACAGCCATCACCGGGG
GATGAGGGCACCTACATCTGCTCCGTGTTCCTGCCCCACGGGCACACACA
GACAGTGCTGCAGCTCCACGTCTTTGAGCCCCCCAAGGTGACGCTGTCCC
CGAAGAACCTGGTGGTGGCTCCGGGGATGTCAGCAGAGCTACGCTGCCAC
GTGTCTGGCTTCTACCCCTTGGATGTGACGGTGACGTGGCAGCGCCGCGC
CGGGAGCTCGGGGACATCACAGTCACCCAGGGACACAGTGATGGACAGCT
GGACTTCAGGTCACCGCCAGGCAGCCGATGGAACCTACAGCCGGACGGCG
GCAGCACGGCTGATCCCCGCACGCCCCCAACACCACGGGGACGTCTACAG
CTGCGTTGTCACCCACACTGCACTGGCCAAACCAATGCGCGTCTCCGTCC
GACTACTCCTGGCTGGCACCGAGGGACCGCACCTGGAGGACATCACGGGG
CTCTTCTTGGTGGCCTTTGTCCTCTGTGGCCTCATCCGTTGGCTCTACCC
TAAAGCTGCACGACCCAAAGAGGAAACCAAGAAATCGCAGTGA
SEQ ID NO. 29: Tapasin coding region (cDNA) in amino acids (B21 haplotype,
from Met to stop)
MAAGLRLLLAGLCWSQFRVEDAASPPPPPAPVRCALLEGVGRRGGLPGGGNARPALLRFGGDAETPPEPGPEPEVTF
NVSDPWGTLTPLGVPPRTPPSCELNPTNPQTGSDPWSRPLHPDARSPPTAGGQWWVAAVGTPQYGVTALLQGGMGTE
GTITAAVALAVLTHTPALRARVGSPTHLHCAFAAPPSPFVLEWRHQNRGAGRVLLAYDSSTARAPRATPGAELLLGT
RDGDGVTAVTLRLAQPSPGDEGTYICSVFLPHGHTQTVLQLHVFEPPKVTLSPKNLVVAPGMSAELRCHVSGFYPLD
VTVTWQRRAGSSGTSQSPRDTVMDSWTSGHRQAADGTYSRTAAARLIPARPQHHGDVYSCVVTHTALAKPMRVSVRL
LLAGTEGPHLEDITGLFLVAFVLCGLIRWLYPKAARPKEETKKSQ*
SEQ ID NO. 30: Promoter and 5′UT for class I major gene (B21 haplotype, B-
F2*2101 from line N)
CCCGTGCGGA CACAGCCCGG GTGCCCCCCC GTGCTCGAAG GGCCGGGGGT TCCCACACCG CGCCCATCCC
CTCCCCCGCT CCGCGCTTTC GCTTTCGCTT CACAACCTGA GGGAGCGCAT TCTGCCTGGC GCCCGATGAC
GTCACATAAG ACTCCAACTA CCATTGGCGG AGAGGCGACG GAGGAGCCAA TGGGGGCGCG GGGCGGGGCG
GAGGAGTAGG AAAAGCTGAA GGAGCTGCGC TGGGTGCGGC GGACTTGAGA GTGCAGCGGT GCGAGGCGAT
GGCTCCGTGC GTGGCGCTGG GCCTGGGGCT GGGGCTGCTG CTCGCCGCCG TGTGCGGGGC GGCGGCCGGT
GAGTGCGGCC GGACCGAGAC GCCTCCCCGC CCATAACCCC ACCCCGGGGC TGTGTCCGTG GGATCCTCAG
ACCCCCACCT GCGGCTCACG GCCCCGCTGC GCTCCGTCCC CGCAGAGCTC CATACCCTGC GGTACAT
SEQ ID NO. 31: Promoter and 5′UT for class I minor gene (B21 haplotype, B-
F1*2101 from line N)
AGTTCTGCTC AGTGCCTCCA TAAGTGATCT GGATAGGTCA TGAGTCATCC TAATTAAGGA GGGGACAACA
GTGAATGGGG AGGAGCCGAT GACTCAGGCT GGGAGTGGTG ATCCCAGAGG TTTCCTCTGC TGTCAGTGAC
TCCGTGCCTC ACCGAGTCCG ACGGCTCCGT GCAGGAAGGA GCGCTCCGCG CACCCCACGC CCCCACCCGG
GGGGGTTCAG CGCAGCCCCC CGGCCGTGCT CTGACAGACG ACTGCTTGCC TTCCCATTGC CCCCCGTCGC
CCCGACAGAG GCCCTCGGCG CCCTCCCCTG GGTTAGGAAC ACGGCCCCGG GGGGGACACA GCCCGAGTGC
CCTTTCGGTG TCCGGGAGGC AGGGAGGGGA CCCCCACCGC GCCCGTCCCC GCCCCCGACT CCGCGCTTTC
GCTTTCGCTT CACAACCTGA GGGAGCGCAT TCTGCCTGGC GCCCGATGAC GTCTCGCGCG CTCCCGGTCG
CCATTGGCGG GGCGGCAACG GAGGAACCAA TGGGGGCGC GGGTGCGGGAC GGGGACTGGT CCCAGAACGT
GGAGGAGCGG TGCGGTGCGG GGCGATGCGC CCGTGCGGG GCGGTGGGCCT GGGGCTGCTG CGCCTGGGGC
TGCTGCTCGC CGCCGTGTGC GGGGCGGCGG CCGGGTGTG CGGCCGGACCG GGACCCCTCC CCGCCCGTAA
CCCCACCCCG GGGCTGTGCC CGTGGGATCC TCAGACCCA CACCCGCGGCT CACGGCCCCG NTGCGCTCCG
TCCCCGCAGA GCTCCATACC CTGCGGTACA T
SEQ ID NO. 32: pONY8.4GCZ
AGATCTTGAATAATAAAATGTGTGTTTGTCCGAAATACGCGTTTTGAGATTTCTGTCGCC
GACTAAATTCATGTCGCGCGATAGTGGTGTTTATCGCCGATAGAGATGGCGATATTGGAA
AAATTGATATTTGAAAATATGGCATATTGAAAATGTCGCCGATGTGAGTTTCTGTGTAAC
TGATATCGCCATTTTTCCAAAAGTGATTTTTGGGCATACGCGATATCTGGCGATAGCGCT
TATATCGTTTACGGGGGATGGCGATAGACGACTTTGGTGACTTGGGCGATTCTGTGTGTC
GCAAATATCGCAGTTTCGATATAGGTGACAGACGATATGAGGCTATATCGCCGATAGAGG
CGACATCAAGCTGGCACATGGCCAATGCATATCGATCTATACATTGAATCAATATTGGCC
ATTAGCCATATTATTCATTGGTTATATAGCATAAATCAATATTGGCTATTGGCCATTGCA
TACGTTGTATCCATATCGTAATATGTACATTTATATTGGCTCATGTCCAACATTACCGCC
ATGTTGACATTGATTATTGACTAGTTATTAATAGTAATCAATTACGGGGTCATTAGTTCA
TAGCCCATATATGGAGTTCCGCGTTACATAACTTACGGTAAATGGCCCGCCTGGCTGACC
GCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGT
ACATCAAGTGTATCATATGCCAAGTCCGCCCCCTATTGACGTCAATGACGGTAAATGGCC
CGCCTGGCATTATGCCCAGTACATGACCTTACGGGACTTTCCTACTTGGCAGTACATCTA
CGTATTAGTCATCGCTATTACCATGGTGATGCGGTTTTGGCAGTACACCAATGGGCGTGG
ATAGCGGTTTGACTCACGGGGATTTCCAAGTCTCCACCCCATTGACGTCAATGGGAGTTT
GTTTTGGCACCAAAATCAACGGGACTTTCCAAAATGTCGTAACAACTGCGATCGCCCGCC
CCGTTGACGCAAATGGGCGGTAGGCGTGTACGGTGGGAGGTCTATATAAGCAGAGCTCGT
TTAGTGAACCGGGCACTCAGAYFCTGGGGTCTGAGTCCCTTCTCTGCTGGGCTGAAAAGG
CCTTTGTAATAAATATAATTCTCTACTCAGTCCCTGTCTCTAGTTTGTCTGTTCGAGATC
CTACAGTTGGCGCCCGAACAGGGACCTGAGAGGGGCGCAGACCCTACCTGTTGAACCTGG
CTGATCGTAGGATCCCCGGGACAGCAGAGGAGAACTTACAGAAGTCTTCTGGAGGTGTTC
CTGGCCAGAACACAGGAGGACAGGTAAGATTGGGAGACCCTTTGACATTGGAGCAAGGCG
CTCAAGAAGTTAGAGAAGGTGACGGTACAAGGGTCTGAGAAATTAACTACTGGTAACTGT
AATTGGGCGCTAAGTCTAGTAGACTTATTTCATTGATACCAACTTTGTAAAAGAAAAGGA
CTGGCAGCTGAGGGATTGTCATTCCATTGCTGGAAGATTGTAACTCAGACGCTGTCAGGA
CAAGAAAGAGAGGCCTTTGAAAGAACATTGGTGGGCAATTTCTGCTGTAAAGATTGGGCC
TCCAGATTAATAATTGTAGTAGATTGGAAAGGCATCATTCCAGCTCCTAAGAGCGAAATA
TTGAAAAGAAGACTGCTAATAAAAAGCAGTCTGAGCCCTCTGAAGAATATCTCTAGAGTC
GACGGTACCGCGGGCCCGGGATCCACCGGTCGCCACCATGGTGAGCAAGGGCGAGGAGCT
GTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCAT
CTGCACCACCGGCAAGCTGCCCGTGCCCTGGCCCACCCTCGTGACCACCCTGACCTACGG
CGTGCAGTGCTTCAGCCGCTACCCCGACCACATGAAGCAGGACGACTTCTTCAAGTCCGC
CATGCCCGAAGGCTACGTCCAGGAGCGCACCATCTTCTTCAAGGACGACGGCAACTACAA
GACCCGCGCCGAGGTGAAGTTCGAGGGCGACACCCTGGTGAACCGCATCGAGCTGAAGGG
CATCGACTTCAAGGAGGACGGCAACATCCTGGGGCACAAGCTGGAGTACAACTACAACAG
CCACAACGTCTATATCATGGCCGACAAGCAGAAGAACGGCATCAAGGTGAACTTCAAGAT
CCGCCACAACATCGAGGACGGCAGCGTGCAGCTCGCCGACCACTACCAGCAGAACACCCC
CATCGGCGACGGCCCCGTGCTGCTGCCCGACAACCACTACCTGAGCACCCAGTCCGCCCT
GAGCAAAGACCCCAACGAGAAGCGCGATCACATGGTCCTGCTGGAGTTCGTGACCGCCGC
CGGGATCACTCTCGGCATGGACGAGCTGTACAAGTAAAGCGGCCGCTCTAGAACTAGTGG
ATCCCCCGGGCTGCAGGAGTGGGGAGGCACGATGGCCGCTTTGGTCGAGGCGGATCCGGC
CATTAGCCATATTATTCATTGGTTATATAGCATAAATCAATATTGGCTATTGGCCATTGC
ATACGTTGTATCCATATCATAATATGTACATTTATATTGGCTCATGTCCAACATTACCGC
CATGTTGACATTGATTATTGACTAGTTATTAATAGTAATCAATTACGGGGTCATTAGTTC
ATAGCCCATATATGGAGTTCCGCGTTACATAACTTACGGTAAATGGCCCGCCTGGCTGAC
CGCCCAACGACCCCCGCCCATTGACGTCAATAATGACGTATGTTCCCATAGTAACGCCAA
TAGGGACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAAATGGC
CCGCCTGGCATTATGCCCAGTACATGACCTTATGGGACTTTCCTACTTGGCAGTACATCT
ACGTATTAGTCATCGCTATTACCATGGTGATGGGGTTTTGGCAGTACATCAATGGGCGTG
GATAGCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCATTGA
CGCAAATGGGCGGTAGGCATGTACGGTGGGAGGTCTATATAAGCAGAGCTCGTTTAGTGA
ACCGTCAGATCGCCTGGAGACGCCATCCACGCTGTTTTGACCTCCATAGAAGACACCGGG
ACCGATCCAGCCTCCGCGGCCCCAAGCTTCAGCTGCTCGAGGATCTGCGGATCCGGGGAA
TTCCCCAGTCTCAGGATCCACCATGGGGGATCCCGTCGTTTTACAACGTCGTGACTGGGA
AAACCCTGGCGTTACCCAACTTAATCGCCTTGCAGCACATCCCCCTTTCGCCAGCTGGCG
TAATAGCGAAGAGGCCCGCACCGATCGCCCTTCCCAACAGTTGCGCAGCCTGAATGGCGA
ATGGCGCTTTGCCTGGTTTCCGGCACCAGAAGCGGTGCCGGAAAGCTGGCTGGAGTGCGA
TCTTCCTGAGGCCGATACTGTCGTCGTCCCCTCAAACTGGCAGATGCACGGTTACGATGC
GCCCATCTACACCAACGTAACCTATCCCATTACGGTCAATCCGCCGTTTGTTCCCACGGA
GAATCCGACGGGTTGTTACTCGCTCACATTTAATGTTGATGAAAGCTGGCTACAGGAAGG
CCAGACGCGAATTATTTTTGATGGCGTTAACTCGGCGTTTCATCTGTGGTGCAACGGGCG
CTGGGTCGGTTACGGCCAGGACAGTCGTTTGCCGTCTGAATTTGACCTGAGCGCATTTTT
ACGCGCCGGAGAAAACCGCCTCGCGGTGATGGTGCTGCGTTGGAGTGACGGCAGTTATCT
GGAAGATCAGGATATGTGGCGGATGAGCGGCATTTTCCGTGACGTCTCGTTGCTGCATAA
ACCGACTACACAAATCAGCGATTTCCATGTTGCCACTCGCTTTAATGATGATTTCAGCCG
CGCTGTACTGGAGGCTGAAGTTCAGATGTGCGGCGAGTTGCGTGACTACCTACGGGTAAC
AGTTTCTTTATGGCAGGGTGAAACGCAGGTCGCCAGCGGCACCGCGCCTTTCGGCGGTGA
AATTATCGATGAGCGTGGTGGTTATGCCGATCGCGTCACACTACGTCTGAACGTCGAAAA
CCCGAAACTGTGGAGCGCCGAAATCCCGAATCTCTATCGTGCGGTGGTTGAACTGCACAC
CGCCGACGGCACGCTGATTGAAGCAGAAGCCTGCGATGTCGGTTTCCGCGAGGTGCGGAT
TGAAAATGGTCTGCTGCTGCTGAACGGCAAGCCGTTGCTGATTCGAGGCGTTAACCGTCA
CGAGCATCATCCTCTGCATGGTCAGGTCATGGATGAGCAGACGATGGTGCAGGATATCCT
GCTGATGAAGCAGAACAACTTTAACGCCGTGCGCTGTTCGCATTATCCGAACCATCCGCT
GTGGTACACGCTGTGCGACCGCTACGGCCTGTATGTGGTGGATGAAGCCAATATTGAAAC
CCACGGCATGGTGCCAATGAATCGTCTGACCGATGATCCGCGCTGGCTACCGGCGATGAG
CGAACGCGTAACGCGAATGGTGCAGCGCGATCGTAATCACCCGAGTGTGATCATCTGGTC
GCTGGGGAATGAATCAGGCCACGGCGCTAATCACGACGCGCTGTATCGCTGGATCAAATC
TGTCGATCCTTCCCGCCCGGTGCAGTATGAAGGCGGCGGAGCCGACACCACGGCCACCGA
TATTATTTGCCCGATGTACGCGCGCGTGGATGAAGACCAGCCCTTCCCGGCTGTGCCGAA
ATGGTCCATCAAAAAATGGCTTTCGCTACCTGGAGAGACGCGCCCGCTGATCCTTTGCGA
ATACGCCCACGCGATGGGTAACAGTCTTGGCGGTTTCGCTAAATACTGGCAGGCGTTTCG
TCAGTATCCCCGTTTACAGGGCGGCTTCGTCTGGGACTGGGTGGATCAGTCGCTGATTAA
ATATGATGAAAACGGCAACCCGTGGTCGGCTTACGGCGGTGATTTTGGCGATACGCCGAA
CGATCGCCAGTTCTGTATGAACGGTCTGGTCTTTGCCGACCGCACGCCGCATCCAGCGCT
GACGGAAGCAAAACACCAGCAGCAGTTTTTCCAGTTCCGTTTATCCGGGCAAACCATCGA
AGTGACCAGCGAATACCTGTTCCGTCATAGCGATAACGAGCTCCTGCACTGGATGGTGGC
GCTGGATGGTAAGCCGCTGGCAAGCGGTGAAGTGCCTCTGGATGTCGCTCCACAAGGTAA
ACAGTTGATTGAACTGCCTGAACTACCGCAGCCGGAGAGCGCCGGGCAACTCTGGCTCAC
AGTACGCGTAGTGCAACCGAACGCGACCGCATGGTCAGAAGCCGGGCACATCAGCGCCTG
GCAGCAGTGGCGTCTGGCGGAAAACCTCAGTGTGACGCTCCCCGCCGCGTCCCACGCCAT
CCCGCATCTGACCACCAGCGAAATGGATTTTTGCATCGAGCTGGGTAATAAGCGTTGGCA
ATTTAACCGCCAGTCAGGCTTTCTTTCACAGATGTGGATTGGCGATAAAAAACAACTGCT
GACGCCGCTGCGCGATCAGTTCACCCGTGCACCGCTGGATAACGACATTGGCGTAAGTGA
AGCGACCCGCATTGACCCTAACGCCTGGGTCGAACGCTGGAAGGCGGCGGGCCATTACCA
GGCCGAAGCAGCGTTGTTGCAGTGCACGGCAGATACACTTGCTGATGCGGTGCTGATTAC
GACCGCTCACGCGTGGCAGCATCAGGGGAAAACCTTATTTATCAGCCGGAAAACCTACCG
GATTGATGGTAGTGGTCAAATGGCGATTACCGTTGATGTTGAAGTGGCGAGCGATACACC
GCATCCGGCGCGGATTGGCCTGAACTGCCAGCTGGCGCAGGTAGCAGAGCGGGTAAACTG
GCTCGGATTAGGGCCGCAAGAAAACTATCCCGACCGCCTTACTGCCGCCTGTTTTGACCG
CTGGGATCTGCCATTGTCAGAGATGTATACCCCGTACGTCTTCCCGAGCGAAAACGGTCT
GCGCTGCGGGACGCGCGAATTGAATTATGGCCCACACCAGTGGCGCGGCGACTTCCAGTT
CAACATCAGCCGCTACAGTCAACAGCAACTGATGGAAACCAGCCATCGCCATCTGCTGCA
CGCGGAAGAAGGCACATGGCTGAATATCGACGGTTTCCATATGGGGATTGGTGGCGACGA
CTCCTGGAGCCCGTCAGTATCGGCGGAATTCCAGCTGAGCGCCGGTCGCTACCATTACCA
GTTGGTCTGGTGTCAAAAATAATAATAACCGGGCAGGGGGGATCCGCAGATCCGGCTGTG
GAATGTGTGTCAGTTAGGGTGTGGAAAGTCCCCAGGCTCCCCAGCAGGCAGAAGTATGCA
AAGCATGCCTGCAGGAATTCGATATCAAGCTTATCGATACCGTCGAATTGGAAGAGCTTT
AAATCCTGGCACATCTCATGTATCAATGCCTCAGTATGTTTAGAAAAACAAGGGGGGAAC
TGTGGGGTTTTTATGAGGGGTTTTATAAAAATGAAAGACCCCACCTGTAGGTTTGGCAAG
CTAGCTTAAGTAAGGCGATTTTGCAAGGCATGGAAAAATACATAACTGAGAATAGAGAAG
TTCAGATCAAGGTCAGGAACAGATGGAACAGCTGAATATGGGCCAAACAGGATATCTGTG
GTAAGCAGTTCCTGCCCCGGCTCAGGGCCAAGAACAGATGGAACAGCTGAATATGGGCCA
AACAGGATATCTGTGGTAAGCAGTTCCTGCCCCGGCTCAGGGCCAAGAACAGATGGTCCC
CAGATGCGGTCCAGCCCTCAGCAGTTTCTAGAGAACCATCAGATGTTTCCAGGGTGCCCC
AAGGACCTGAAATGACCCTGTGCCTTATTTGAACTAACCAATCAGTTCGCTTCTGGCTTG
TGTTCGCGCGCTTCTGCTCCCCGAGCTCAATAAAAGAGCCCACAACCCCTCACTCGGGGG
GCACTCAGATTCTGCGGTCTGAGTCCCTTCTGTGCTGGGCTGAAAAGGCCTTTGTAATAA
ATATAATTCTCTACTCAGTCCCTGTCTCTAGTTTGTCTGTTCGAGATCCTACAGAGCTCA
TGCCTTGGCGTAATCATGGTCATAGCTGTTTCCTGTGTGAAATTGTTATCCGCTCACAAT
TCCACACAACATACGAGCCGGAAGCATAAAGTGTAAAGCCTGGGGTGCCTAATGAGTGAG
CTAACTCACATTAATTGCGTTGCGCTCACTGCCCGCTTTCCAGTCGGGAAACCTGTCGTG
CCAGCTGCATTAATGAATCGGCCAACGCGCGGGGAGAGGCGGTTTGCGTATTGGGCGCTC
TTCCGCTTCCTCGCTCACTGACTCGCTGCGCTCGGTCGTTCGGCTGCGGCGAGCGGTATC
AGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCTCGTGCG
CTCTCCTGTTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTCGTTCGCTC
CAAGCTGGGCTGTGTGCACGAACCCCCCGTTCAGCCCGACCGCTGCGCCTTATCCGGTAA
CTATCGTCTTGAGTCCAACCCGGTAAGACACGACTTATCGCCACTGGCAGCAGCCACTGG
TAACAGGATTAGCAGAGCGAGGTATGTAGGCGGTGCTACAGAGTTCTTGAAGTGGTGGCC
TAACTACGGCTACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAA
ATCAATCTAAAGTATATATGAGTAAACTTGGTCTGACAGTTACCAATGCTTAATCAGTGA
GGCACCTATCTCAGCGATCTGTCTATTTCGTTCATCCATAGTTGCCTGACTCCCCGTCGT
GTAGATAACTACGATACGGGAGGGCTTACCATCTGGCCCCAGTGCTGCAATGATACCGCG
AGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGA
AGCTAGAGTAAGTAGTTCGCCAGTTAATAGTTTGCGCAACGTTGTTGCCATTGCTACAGG
CATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCC
GATCGTTGTCAGAAGTAAGTTGGCCGCAGTGTTATCACTCATGGTTATGGCAGCACTGCA
TAATTCTCTTACTGTCATGCCATCCGTAAGATGCTTTTCTGTGACTGGTGAGTACTCAAC
CAAGTCATTCTGAGAATAGTGTATGCGGCGACCGAGTTGCTCTTGCCCGGCGTCAATACG
GGATAATACCGCGCCACATAGCAGAACTTTAAAAGTGCTCATCATTGGAAAACGTTCTTC
GGGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAA
AGTGCCACCTAAATTGTAAGCGTTAATATTTTGTTAAAATTCGCGTTAAATTTTTGTTAA
ATCAGCTCATTTTTTAACCAATAGGCCGAAATCGGCAAAATCCCTTATAAATCAAAAGAA
TAGACCGAGATAGGGTTGAGTGTTGTTCCAGTTTGGAACAAGAGTCCACTATTAAAGAAC
GTGGACTCCAACGTCAAAGGGCGAAAAACCGTCTATCAGGGCGATGGCCCACTACGTGAA
CCATCACCCTAATCAAGTTTTTTGGGGTCGAGGTGCCGTAAAGCACTAAATCGGAACCCT
AAAGGGAGCCCCCGATTTAGAGCTTGACGGGGAAAGCCAACCTGGCTTATCGAAATTAAT
ACGACTCACTATAGGGAGACCGGC
SEQ ID NO. 33: 5′ primer
aaaaaaaagctTCTTGAGAGTGCAGCGGTGCGAGG
SEQ ID NO. 34: 3′ primer
aaaaaaaagcttCACAGAATGGCCCATCATTTTATTTC
SEQ ID NO. 35: pONY8.4GCMCHI
AGATCTTGAATAATAAAATGTGTGTTTGTCCGAAATACGCGTTTTGAGATTTCTGTCGCC
GACTAAATTCATGTCGCGCGATAGTGGTGTTTATCGCCGATAGAGATGGCGATATTGGAA
AAATTGATATTTGAAAATATGGCATATTGAAAATGTCGCCGATGTGAGTTTCTGTGTAAC
TGATATCGCCATTTTTCCAAAAGTGATTTTTGGGCATACGCGATATCTGGCGATAGCGCT
TATATCGTTTACGGGGGATGGCGATAGACGACTTTGGTGACTTGGGCGATTCTGTGTGTC
GCAAATATCGCAGTTTCGATATAGGTGACAGACGATATGAGGCTATATCGCCGATAGAGG
CGACATCAAGCTGGCACATGGCCAATGCATATCGATCTATACATTGAATCAATATTGGCC
ATTAGCCATATTATTCATTGGTTATATAGCATAAATCAATATTGGCTATTGGCCATTGCA
TACGTTGTATCCATATCGTAATATGTACATTTATATTGGCTCATGTCCAACATTACCGCC
ATGTTGACATTGATTATTGACTAGTTATTAATAGTAATCAATTACGGGGTCATTAGTTCA
TAGCCCATATATGGAGTTCCGCGTTACATAACTTACGGTAAATGGCCCGCCTGGCTGACC
GCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGT
ACATCAAGTGTATCATATGCCAAGTCCGCCCCCTATTGACGTCAATGACGGTAAATGGCC
CGCCTGGCATTATGCCCAGTACATGACCTTACGGGACTTTCCTACTTGGCAGTACATCTA
CGTATTAGTCATCGCTATTACCATGGTGATGCGGTTTTGGCAGTACACCAATGGGCGTGG
ATAGCGGTTTGACTCACGGGGATTTCCAAGTCTCCACCCCATTGACGTCAATGGGAGTTT
GTTTTGGCACCAAAATCAACGGGACTTTCCAAAATGTCGTAACAACTGCGATCGCCCGGC
CCGTTGACGCAAATGGGCGGTAGGCGTGTACGGTGGGAGGTCTATATAAGCAGAGCTCGT
TTAGTGAACCGGGCACTCAGATTCTGCGGTCTGAGTCCCTTCTCTGCTGGGCTGAAAAGG
CCTTTGTAATAAATATAATTCTCTACTCAGTCCCTGTCTCTAGTTTGTCTGTTCGAGATC
CTACAGTTGGCGCCCGAACAGGGACCTGAGAGGGGCGCAGACCCTACCTGTTGAACCTGG
CTGATCGTAGGATCCCCGGGACAGCAGAGGAGAACTTACAGAAGTCTTCTGGAGGTGTTC
CTGGCCAGAACACAGGAGGACAGGTAAGATTGGGAGACCCTTTGACATTGGAGCAAGGCG
CTCAAGAAGTTAGAGAAGGTGACGGTACAAGGGTCTCAGAAATTAACTACTGGTAACTGT
AATTGGGCGCTAAGTCTAGTAGACTTATTTCATTGATACCAACTTTGTAAAAGAAAAGGA
CTGGCAGCTGAGGGATTGTCATTCCATTGCTGGAAGATTGTAACTCAGACGCTGTCAGGA
CAAGAAAGAGAGGCCTTTGAAAGAACATTGGTGGGCAATTTCTGCTGTAAAGATTGGGCC
TCCAGATTAATAATTGTAGTAGATTGGAAAGGCATCATTCCAGCTCCTAAGAGCGAAATA
TTGAAAAGAAGACTGCTAATAAAAAGCAGTCTGAGCCCTCTGAAGAATATCTCTAGAGTC
GACGGTACCGCGGGCCCGGGATCCACCGGTCGCCACCATGGTGAGCAAGGGCGAGGAGCT
GTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCAT
CTGCACCACCGGCAAGCTGCCCGTGCCCTGGCCCACCCTCGTGACCACCCTGACCTACGG
CGTGCAGTGCTTCAGCCGGTACCCCGACCACATGAAGCAGCACGACTTCTTCAAGTCCGC
CATGCCCGAAGGCTACGTCCAGGAGCGCACCATCTTCTTCAAGGACGACGGCAACTACAA
GACCCGCGCCGAGGTGAAGTTCGAGGGCGACACCCTGGTGAACCGCATCGAGCTGAAGGG
CATCGACTTCAAGGAGGACGGCAACATCCTGGGGCACAAGCTGGAGTACAACTACAACAG
CCACAACGTCTATATCATGGCCGACAAGCAGAAGAACGGCATCAAGGTGAACTTCAAGAT
CCGCCACAACATCGAGGACGGCAGCGTGCAGCTCGCCGACCACTACCAGCAGAACACCCC
CATCGGCGACGGCCCCGTGCTGCTGCCCGACAACCACTACCTGAGCACCCAGTCCGCCCT
GAGCAAAGACCCCAACGAGAAGCGCGATCACATGGTCCTGCTGGAGTTCGTGACCGCCGC
CGGGATCACTCTCGGCATGGACGAGCTGTACAAGTAAAGCGGCCGCTCTAGAACTAGTGG
ATCCCCCGGGCTGCAGGAGTGGGGAGGCACGATGGCCGCTTTGGTCGAGGCGGATCCGGC
CATTAGCCATATTATTCATTGGTTATATAGCATAAATCAATATTGGCTATTGGCCATTGC
ATACGTTGTATCCATATCATAATATGTACATTTATATTGGCTCATGTCCAACATTACCGC
CATGTTGACATTGATTATTGACTAGTTATTAATAGTAATCAATTACGGGGTCATTAGTTC
ATAGCCCATATATGGAGTTCCGCGTTACATAACTTACGGTAAATGGCCCGCCTGGCTGAC
CGCCCAACGACCCCCGCCCATTGACGTCAATAATGACGTATGTTCCCATAGTAACGCCAA
TAGGGACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAAATGGC
CCGCCTGGCATTATGCCCAGTACATGACCTTATGGGACTTTCCTACTTGGCAGTACATCT
ACGTATTAGTCATCGCTATTACCATGGTGATGCGGTTTTGGCAGTACATCAATGGGCGTG
GATAGCGGTTTGACTCAGGGGGATTTCCAAGTCTCCACCCCATTGACGTCAATGGGAGTT
TGTTTTGGCACCAAAATCAACGGGACTTTCCAAAATGTCGTAACAACTCCGCCCCATTGA
CGCAAATGGGCGGTAGGCATGTACGGTGGGAGGTCTATATAAGCAGAGCTCGTTTAGTGA
ACCGTCAGATCGCCTGGAGACGCCATCCACGCTGTTTTGACCTCCATAGAAGACACCGGG
ACCGATCCAGCCTCCGCGGCCCCAAGCTTCTTGAGAGTGCAGCGGTGCGAGGCGATGGGC
TCGTGCGGGGGGCTGGGCGTGGGGCTGCTGCTCGCCGCCGTGTGCGGGGCGGCGGCCGAG
CTCCATACCCTGCGGTACATCCGTACGGCGATGACGGATCCCGGCCCCGGGCTGCCGTGG
TTCGTGGACGTGGGGTACGTGGACGGGGAACTCTTCATGCAGTACAACAGCACCGCTCGG
AGGGCTGTGCCCCGCACCGAGTGGATAGCGGCCAACACGGACCAGCAGTACTGGGACAGA
GAGACGCAGATCGTACAGGGCAGTGAGCAGATTAACCGCGAGAACCTGGACATACTGCGG
CGGCGCTACAACCAGACCGGCGGGTCTCACACAGTGCAGTGGATGTCCGGCTGTGACATC
CTCGAGGATGGCACCATCCGGGGGTATCATCAGGCAGCCTACGATGGGAGAGACTTCGTT
GCCTTCGACAAAGGCACGATGACGTTAACTGCGGCAGTTCCAGAGGCAGTTCCCACCAAG
AGGAAATGGGAGGAAGGAGGTTATGCTGAGGGGCTGAAGCAGTACCTGGAGGAAACCTGC
GTGGAGTGGCTGCGGAGATATGTGGAATACGGGAAGGCTGAGCTGGGCAGGAGAGAGCGA
CCCGAGGTGCGAGTGTGGGGGAAGGAGGCCGACGGGATCCTGACCTTGTCCTGCCGCGCT
CACGGCTTCTACCCGCGGCCCATCGTTGTCAGCTGGCTGAAGGACGGCGCGGTGGGGGGC
CAGGACGCCCAGTCGGGGGGCATCGTGCCCAACGGCGACGGCACGTACCACACCTGGGTC
ACCATCGATGCGCAGCCGGGGGACGGGGACAAGTACCAGTGCCGCGTGGAGCACGCCAGC
CTGCCCCAGCCCGGCCTCTACTCGTGGGAGCCGCCACAGCCCAACCTGGTGCCCATCGTG
GCGGGGGTGGCTGTCGCCATTGTGGCCATCGCCATCGTGGTTGGTGTTGGATTCATCATC
TACAGACGCCATGCAGGGAAGAAGGGGAAGGGCTACAACATCGCGCCCGACAGGGAAGGT
GGATCCAGCAGCTCGAGCACAGGGAGCAACCCCTCCATGTGAGTGCTGTGCTTCAGCCTG
CAAGGAGCCAACAGTCCACACCAGCATTTGGGGTCGGTGATGGACACAGCCCCATCCTCT
TGACCTCTCAGATGTCCCTCTGCTTCCTATGCTGACTGTTATTCTTTGCCTGCACTGCTT
CCTGTGAAATAAAATGATGGGCCATTCTGTGAAGCTTATCGATACCGTCGAATTGGAAGA
GCTTTAAATCCTGGCACATCTCATGTATCAATGCCTCAGTATGTTTAGAAAAACAAGGGG
GGAACTGTGGGGTTTTTATGAGGGGTTTTATAAAAATGAAAGACCCCACCTGTAGGTTTG
GCAAGCTAGCTTAAGTAACGCCATTTTGCAAGGCATGGAAAAATACATAACTGAGAATAG
AGAAGTTCAGATCAAGGTCAGGAACAGATGGAACAGCTGAATATGGGCCAAACAGGATAT
CTGTGGTAAGCAGTTCCTGCCCCGGCTCAGGGCCAAGAACAGATGGAACAGCTGAATATG
GGCCAAACAGGATATCTGTGGTAAGCAGTTCCTGCCCCGGCTCAGGGCCAAGAACAGATG
GTCCCCAGATGCGGTCCAGCCCTCAGGAGTTTCTAGAGAACCATCAGATGTTTCCAGGGT
GCCCCAAGGACCTGAAATGACCCTGTGCCTTATTTGAACTAACCAATCAGTTCGCTTCTC
GCTTCTGTTCGCGCGCTTCTGCTCCCCGAGCTCAATAAAAGAGCCCACAACCCCTCACTC
GGGGGGCACTCAGATTCTGCGGTCTGAGTCCCTTCTCTGCTGGGCTGAAAAGGCCTTTGT
AATAAATATAATTCTCTACTCAGTCCCTGTCTCTAGTTTGTCTGTTCGAGATCCTACAGA
GCTCATGCCTTGGCGTAATCATGGTCATAGCTGTTTCCTGTGTGAAATTGTTATCCGCTC
ACAATTCCACACAACATACGAGCCGGAAGCATAAAGTGTAAAGCCTGGGGTGCCTAATGA
GTGAGCTAACTCACATTAATTGCGTTGCGCTCACTGCCCGCTTTCCAGTCGGGAAACCTG
TCGTGCCAGCTGCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGATAACGCAGGA
AAGAACATGTGAGCAAAAGGCCAGCAAAAGGCCAGGAACCGTAAAAAGGCCGCGTTGCTG
GCGTTTTTCCATAGGCTCCGCCCCCCTGACGAGCATCACAAAAATCGACGCTCAAGTCAG
AGGTGGCGAAACCCGACAGGACTATAAAGATACCAGGCGTTTCCCCCTGGAAGCTCCCTC
GTGCGCTCTCCTGTTCCGACCCTGCGGCTTACCGGATACCTGTCCGCCTTTGTCCCTTCG
GGAAGCGTGGCGCTTTCTCATAGCTCACGCTGTAGGTATCTCAGTTCGGTGTAGGTCGTT
CGCTCCAAGCTGGGCTGTGTGCACGAACCCCCCGTTCAGCCCGACCGCTGCGCCTTATCC
GGTAACTATCGTCTTGAGTCCAACCCGGTAAGACACGAGTTATCGGCACTGGCAGCAGCC
ACTGGTAACAGGATTAGCAGAGCGAGGTATGTAGGCGGTGCTACAGAGTTCTTGAAGTGG
TGGCCTAACTACGGCTACACTAGAAGGACAGTATTTGGTATCTGCGCTCTGCTGAAGCCA
GTTACCTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAGAAGAT
CCTTTGATCTTTTCTACGGGGTCTGACGCTCAGTGGAACGAAAACTCACGTTAAGGGATT
TTGGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAATC
AGTGAGGCACCTATCTCAGCGATCTGTCTATTTCGTTCATCCATAGTTGCCTGACTCCCC
GTCGTGTAGATAACTACGATACGGGAGGGCTTACCATCTGGCCCCAGTGCTGCAATGATA
CCGCGAGACCCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATTAATTGTTGC
CGGGAAGCTAGAGTAAGTAGTTCGCCAGTTAATAGTTTGCGCAACGTTGTTGCCATTGCT
ACAGGCATCGTGGTGTCACGCTCGTCGTTTGGTATGGCTTCATTCAGCTCCGGTTCCCAA
CGATCAAGGCGAGTTACATGATCCCCCATGTTGTGGAAAAAAGCGGTTAGCTCCTTCGGT
CCTCCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGTAC
TCAACCAAGTCATTCTGAGAATAGTGTATGCGGCGACCGAGTTGCTCTTGCCCGGCGTCA
ATACGGGATAATACCGCGCCACATAGCAGAACTTTAAAAGTGCTCATCATTGGAAAACGT
TCTTCGGGGCGAAAACTCTCAAGGATCTTACCGCTGTTGAGATCCAGTTCGATGTAACCC
ACTCGTGCACCCAACTGATCTTCAGCATCTTTTACTTTCACCAGCGTTTCTGGGTGAGCA
AAAACAGGAAGGCAAAATGCCGCAAAAAAGGGAATAAGGGCGACACGGAAATGTTGAATA
CTCATACTCTTCCTTTTTCAATATTATTGAAGCATTTATCAGGGTTATTGTCTCATGAGC
GGATACATATTTGAATGTATTTAGAAAAATAAACAAATAGGGGTTCCGCGCACATTTCCC
CGAAAAGTGCCACCTAAATTGTAAGCGTTAATATTTTGTTAAAATTCGCGTTAAATTTTT
GTTAAATCAGCTCATTTTTTAACCAATAGGCCGAAATCGGCAAAATCCCTTATAAATCAA
AAGAATAGACCGAGATAGGGTTGAGTGTTGTTCCAGTTTGGAACAAGAGTCCACTATTAA
AGAACGTGGACTCCAACGTCAAAGGGCGAAAAACCGTCTATCAGGGCGATGGCCCACTAC
GTGAACCATCACCCTAATCAAGTTTTTTGGGGTCGAGGTGCCGTAAAGCACTAAATCGGA
ACCCTAAAGGGAGCCCCCGATTTAGAGCTTGACGGGGAAAGCGAACCTGGCTTATCGAAA
TTAATACGACTCACTATAGGGAGACCGGC
```

1. A method for producing a transgenic bird having modified resistance to a disease pathogen comprising introducing a retroviral vector into a germ line cell of a bird, wherein the vector comprises a polynucleotide sequence which encodes an RNA or protein which modifies the disease resistance of a pathogen in the bird, and wherein the RNA or protein is expressed in at least the germ line of the bird thereby producing the transgenic bird having modified resistance to a disease pathogen.

2. The method according to claim 1, further comprising administering a vaccine to the bird.

3. The method according to claim 1, wherein the polynucleotide sequence encodes at least one RNA or protein which is related to or associated with the major histocompatibility complex (MHC) of the bird.

4. The method according to claim 3, wherein the MHC of the bird is MHC Class I or MHC class II of the bird.

5. The method according to claim 1, wherein the polynucleotide sequence comprises a promoter which is related to or associated with the MHC of the bird.

6. The method according to claim 5, wherein the MHC of the bird is MHC Class I or MHC Class II of the bird.

7. The method according to claim 3, wherein the protein is an MHC accessory protein.

8. The method according to claim 7, wherein the MHC accessory protein is TAP, tapasin, B-lec, B-NK, calnexin (CNX), calrecticulin (CRT), Erp57, DM or DO.

9. The method according to claim 7, wherein the MHC accessory protein is a natural killer (NK) receptor.

10. The method according to claim 1, wherein the polynucleotide sequence encodes a cytokine.

11. The method according to claim 10, wherein the cytokine is INF-alpha, IFN-beta, IFN-gamma, IL-1beta, IL-2, IL-3, IL-4, IL-6, IL-8, IL-10, IL-12beta, IL-13, IL-15, IL-18, TGF-beta4, or GMCSF.

12. The method according to claim 1, wherein the protein is a CD1 protein.

13. The method according to claim 1, wherein the retroviral vector is a lentiviral vector.

14. The method according to claim 13, wherein the lentiviral vector is an HIV-based lentiviral vector or an EIAV-based lentiviral vector.

15. The method according to claim 13, wherein the lentiviral vector is a minimal lentiviral vector.

16. The method according to claim 1, wherein the retroviral vector is a pseudotyped retroviral vector.

17. The method according to claim 1, wherein the polynucleotide sequence comprises a constitutive, tissue-specific, spatial or inducible promoter.

18. The method according to claim 1, wherein the retroviral vector is introduced into the germ line cell of the bird in vivo or ex vivo.

19. The method according to claim 1, wherein the germ line cell is in utero.

20. The method according to claim 1, wherein the germ line cell is a perinatal cell.

21. The method according to claim 1, wherein the germ line cell is an embryonic cell.

22. The method according to claim 1, wherein the germ line cell is a fetal cell.

23. The method according to claim 1, wherein the germ line cell is an oocyte, an oviduct cell, an ovarian cell, an ovum, an oogonium, a zygote, an ES cell, a blastocyte, a spermatocyte, a spermatid, a spermatozoa or a spermatogonia.

24. The method according to claim 1, wherein the retroviral vector is introduced into the germ line cell via the blastoderm, umbilical cord, placenta, amniotic fluid, uterus, or gonads

25. The method according to claim 1, wherein the retroviral vector is introduced via intraperitoneal, intramuscular, intraspinal, intracranial, intravenous, intrarespiratory, gastrointestinal or intrahepatic administration.

26. The method according to claim 1, wherein the bird is a domestic fowl.

27. The method according to claim 26, wherein the domestic fowl is a chicken or turkey.

28. The method according to claim 1, wherein the germ line cell is a fertilized bird egg.

29. The method according to claim 1, further comprising breeding the transgenic bird.

30. The method according to claim 1, wherein the RNA is a short-interfering RNA (siRNA).

31. A transgenic bird produced by the method according to claim 1, wherein the transgenic bird expresses the RNA or protein in at least the germ line of the bird such that the transgenic bird exhibits modified resistance to a disease pathogen.

32. A method for producing an avian cell expressing a disease resistance RNA or protein, comprising introducing a retroviral vector into the cell, wherein the vector comprises a polynucleotide sequence which encodes an RNA or protein which modifies the disease resistance of the cell to a pathogen, wherein the cell is an avian germ line cell or avian somatic cell, and wherein the germ line cell or somatic cell expresses the disease resistance RNA or protein.

33-64. (canceled)
